SEQUENCE LISTING (196 sequences)

SEQ ID NO: 1 - protein, 5 aa, Artificial Sequence
  Generic DAT C5
  MISC_FEATURE (1)..(1): H, N, F, or T; or is absent;
  MISC_FEATURE (2)..(2): W, S, E, or Y; or is absent;
  MISC_FEATURE (3)..(3): L, V, or I;
  MISC_FEATURE (4)..(4): K, I, or R;
  MISC_FEATURE (5)..(5): V;
XXXXX

SEQ ID NO: 2 - protein, 5 aa, Artificial Sequence
  Generic DAT C5
  MISC_FEATURE (1)..(1): N, F, or T; or is absent;
  MISC_FEATURE (2)..(2): S, E, or Y; or is absent;
  MISC_FEATURE (3)..(3): V, L, or I;
  MISC_FEATURE (4)..(4): I, or R;
  MISC_FEATURE (5)..(5): V;
XXXXX

SEQ ID NO: 3 - protein, 5 aa, Artificial Sequence
  Generic DAT C5
  MISC_FEATURE (1)..(1): N, or T; or is absent;
  MISC_FEATURE (2)..(2): S, E, or Y; or is absent;
  MISC_FEATURE (3)..(3): V, or I;
  MISC_FEATURE (4)..(4): I, or R;
  MISC_FEATURE (5)..(5): V;
XXXXX

SEQ ID NO: 4 - protein, 5 aa, Artificial Sequence
  Generic DAT C5
  MISC_FEATURE (1)..(1): N, or F; or is absent;
  MISC_FEATURE (2)..(2): S, E, or Y; or is absent;
  MISC_FEATURE (3)..(3): V, or I;
  MISC_FEATURE (4)..(4): I, or R;
  MISC_FEATURE (5)..(5): V;
XXXXX

SEQ ID NO: 5 - protein, 5 aa, Artificial Sequence
  DAT C5 variant
NSIIV

SEQ ID NO: 6 - protein, 5 aa, Artificial Sequence
  DAT C5 VARIANT
NSIRV

SEQ ID NO: 7 - protein, 5 aa, Artificial Sequence
  DAT C5 VARIANT
NSVIV

SEQ ID NO: 8 - protein, 5 aa, Artificial Sequence
  DAT C5 VARIANT
NSVRV

SEQ ID NO: 9 - protein, 5 aa, Artificial Sequence
  DAT C5 VARIANT
NEIIV

SEQ ID NO: 10 - protein, 5 aa, Artificial Sequence
  DAT C5 VARIANT
NEIRV

SEQ ID NO: 11 - protein, 5 aa, Artificial Sequence
  DAT C5 VARIANT
NEVIV

SEQ ID NO: 12 - protein, 5 aa, Artificial Sequence
  DAT C5 VARIANT
NEVRV

SEQ ID NO: 13 - protein, 5 aa, Artificial Sequence
  DAT C5 VARIANT
NYIIV

SEQ ID NO: 14 - protein, 5 aa, Artificial Sequence
  DAT C5 VARIANT
NYIRV

SEQ ID NO: 15 - protein, 5 aa, Artificial Sequence
  DAT C5 VARIANT
NYVIV

SEQ ID NO: 16 - protein, 5 aa, Artificial Sequence
  DAT C5 VARIANT
NYVRV

SEQ ID NO: 17 - protein, 5 aa, Artificial Sequence
  DAT C5 VARIANT
TSIIV

SEQ ID NO: 18 - protein, 5 aa, Artificial Sequence
  DAT C5 VARIANT
TSIRV

SEQ ID NO: 19 - protein, 5 aa, Artificial Sequence
  DAT C5 VARIANT
TSVIV

SEQ ID NO: 20 - protein, 5 aa, Artificial Sequence
  DAT C5 VARIANT
TSVRV

SEQ ID NO: 21 - protein, 5 aa, Artificial Sequence
  DAT C5 VARIANT
TEIIV

SEQ ID NO: 22 - protein, 5 aa, Artificial Sequence
  DAT C5 VARIANT
TEIRV

SEQ ID NO: 23 - protein, 5 aa, Artificial Sequence
  DAT C5 VARIANT
TEVIV

SEQ ID NO: 24 - protein, 5 aa, Artificial Sequence
  DAT C5 VARIANT
TEVRV

SEQ ID NO: 25 - protein, 5 aa, Artificial Sequence
  DAT C5 VARIANT
TYIIV

SEQ ID NO: 26 - protein, 5 aa, Artificial Sequence
  DAT C5 VARIANT
TYIRV

SEQ ID NO: 27 - protein, 5 aa, Artificial Sequence
  DAT C5 VARIANT
TYVIV

SEQ ID NO: 28 - protein, 5 aa, Artificial Sequence
  DAT C5 VARIANT
TYVRV

SEQ ID NO: 29 - protein, 5 aa, Artificial Sequence
  DAT C5 VARIANT
FSIIV

SEQ ID NO: 30 - protein, 5 aa, Artificial Sequence
  DAT C5 VARIANT
FSIRV

SEQ ID NO: 31 - protein, 5 aa, Artificial Sequence
  DAT C5 VARIANT
FSVIV

SEQ ID NO: 32 - protein, 5 aa, Artificial Sequence
  DAT C5 VARIANT
FSVRV

SEQ ID NO: 33 - protein, 5 aa, Artificial Sequence
  DAT C5 VARIANT
FEIIV

SEQ ID NO: 34 - protein, 5 aa, Artificial Sequence
  DAT C5 VARIANT
FEIRV

SEQ ID NO: 35 - protein, 5 aa, Artificial Sequence
  DAT C5 VARIANT
FEVIV

SEQ ID NO: 36 - protein, 5 aa, Artificial Sequence
  DAT C5 VARIANT
FEVRV

SEQ ID NO: 37 - protein, 5 aa, Artificial Sequence
  DAT C5 VARIANT
FYIIV

SEQ ID NO: 38 - protein, 5 aa, Artificial Sequence
  DAT C5 VARIANT
FYIRV

SEQ ID NO: 39 - protein, 5 aa, Artificial Sequence
  DAT C5 VARIANT
FYVIV

SEQ ID NO: 40 - protein, 5 aa, Artificial Sequence
  DAT C5 VARIANT
FYVRV

SEQ ID NO: 41 - protein, 4 aa, Artificial Sequence
  DAT C5 VARIANT
SIIV

SEQ ID NO: 42 - protein, 4 aa, Artificial Sequence
  DAT C5 VARIANT
SIRV

SEQ ID NO: 43 - protein, 4 aa, Artificial Sequence
  DAT C5 VARIANT
SVIV

SEQ ID NO: 44 - protein, 4 aa, Artificial Sequence
  DAT C5 VARIANT
SVRV

SEQ ID NO: 45 - protein, 4 aa, Artificial Sequence
  DAT C5 VARIANT
EIIV

SEQ ID NO: 46 - protein, 4 aa, Artificial Sequence
  DAT C5 VARIANT
EIRV

SEQ ID NO: 47 - protein, 4 aa, Artificial Sequence
  DAT C5 VARIANT
EVIV

SEQ ID NO: 48 - protein, 4 aa, Artificial Sequence
  DAT C5 VARIANT
EVRV

SEQ ID NO: 49 - protein, 4 aa, Artificial Sequence
  DAT C5 VARIANT
YIIV

SEQ ID NO: 50 - protein, 4 aa, Artificial Sequence
  DAT C5 VARIANT
YIRV

SEQ ID NO: 51 - protein, 4 aa, Artificial Sequence
  DAT C5 VARIANT
YVIV

SEQ ID NO: 52 - protein, 4 aa, Artificial Sequence
  DAT C5 VARIANT
YVRV

SEQ ID NO: 53 - protein, 5 aa, Artificial Sequence
  DAT-C5 variant
NSLRV

SEQ ID NO: 54 - protein, 5 aa, Artificial Sequence
  DAT-C5
HWLKV

SEQ ID NO: 55 - protein, 5 aa, Artificial Sequence
  Class I
IETDV

SEQ ID NO: 56 - protein, 6 aa, Artificial Sequence
  Class I
RRTTPV

SEQ ID NO: 57 - protein, 6 aa, Artificial Sequence
  Class I
YKQTSV

SEQ ID NO: 58 - protein, 5 aa, Artificial Sequence
  Class III
WGESV

SEQ ID NO: 59 - protein, 5 aa, Artificial Sequence
  Class III
KVDSV

SEQ ID NO: 60 - protein, 5 aa, Artificial Sequence
  Class III
GKDYV

SEQ ID NO: 61 - protein, 5 aa, Artificial Sequence
  Class III
RKDYV

SEQ ID NO: 62 - protein, 5 aa, Artificial Sequence
  Class III
TAEMF

SEQ ID NO: 63 - protein, 5 aa, Artificial Sequence
  Class III
QEDGA

SEQ ID NO: 64 - protein, 5 aa, Artificial Sequence
  Class I
IETAV

SEQ ID NO: 65 - protein, 11 aa, Artificial Sequence
  RRTTPV monomer
SNTANRRTTPV

SEQ ID NO: 66 - protein, 30 aa, Artificial Sequence
  GCN4p1
RMKQLEDKVEELLSKNYHLENEVARLKKLV

SEQ ID NO: 67 - protein, 30 aa, Artificial Sequence
  GCN4p1(NQ)
RMKQLEDKVEELLSKQYHLENEVARLKKLV

SEQ ID NO: 68 - protein, 30 aa, Artificial Sequence
  GCN4p1(LI)
RIKQIEDKIEEILSKIYHIENEIARIKKLI

SEQ ID NO: 69 - protein, 29 aa, Artificial Sequence
  CC-Tet
GELAAIKQELAAIKKELAAIKWELAAIKQ

SEQ ID NO: 70 - protein, 29 aa, Artificial Sequence
  CC-Hex2
GEIAKSLKEIAKSLKEIAWSLKEIAKSLK

SEQ ID NO: 71 - protein, 4 aa, Artificial Sequence
  GS3
GGGS

SEQ ID NO: 72 - protein, 5 aa, Artificial Sequence
  GS4
GGGGS

SEQ ID NO: 73 - protein, 6 aa, Artificial Sequence
  GS5
GGGGSG

SEQ ID NO: 74 - protein, 7 aa, Artificial Sequence
  GS6
GGGGSGG

SEQ ID NO: 75 - protein, 40 aa, Artificial Sequence
  GCN4p1(NQ)-GS4-HWLKV
RMKQLEDKVEELLSKQYHLENEVARLKKLVGGGGSHWLKV

SEQ ID NO: 76 - protein, 40 aa, Artificial Sequence
  GCN4p1(NQ)-GS4-NSIRV
RMKQLEDKVEELLSKQYHLENEVARLKKLVGGGGSNSIRV

SEQ ID NO: 77 - protein, 40 aa, Artificial Sequence
  GCN4p1(NQ)-GS4-IEDTV
RMKQLEDKVEELLSKQYHLENEVARLKKLVGGGGSIETDV

SEQ ID NO: 78 - protein, 41 aa, Artificial Sequence
  GCN4p1(NQ)-GS4-RRTTPV
RMKQLEDKVEELLSKQYHLENEVARLKKLVGGGGSRRTTPV

SEQ ID NO: 79 - protein, 41 aa, Artificial Sequence
  GCN4p1(NQ)-GS4-YKQTSV
RMKQLEDKVEELLSKQYHLENEVARLKKLVGGGGSYKQTSV

SEQ ID NO: 80 - protein, 40 aa, Artificial Sequence
  GCN4p1(NQ)-GS4-WGESV
RMKQLEDKVEELLSKQYHLENEVARLKKLVGGGGSWGESV

SEQ ID NO: 81 - protein, 40 aa, Artificial Sequence
  GCN4p1(LI)-GS4-HWLKV
RIKQIEDKIEEILSKIYHIENEIARIKKLIGGGGSHWLKV

SEQ ID NO: 82 - protein, 40 aa, Artificial Sequence
  GCN4p1(LI)-GS4-NSIRV
RIKQIEDKIEEILSKIYHIENEIARIKKLIGGGGSNSIRV

SEQ ID NO: 83 - protein, 40 aa, Artificial Sequence
  GCN4p1(LI)-GS4-IETDV
RIKQIEDKIEEILSKIYHIENEIARIKKLIGGGGSIETDV

SEQ ID NO: 84 - protein, 41 aa, Artificial Sequence
  GCN4p1(LI)-GS4-RRTTPV
RIKQIEDKIEEILSKIYHIENEIARIKKLIGGGGSRRTTPV

SEQ ID NO: 85 - protein, 41 aa, Artificial Sequence
  GCN4p1(LI)-GS4-YKQTSV
RIKQIEDKIEEILSKIYHIENEIARIKKLIGGGGSYKQTSV

SEQ ID NO: 86 - protein, 40 aa, Artificial Sequence
  GCN4p1(LI)-GS4-WGESV
RIKQIEDKIEEILSKIYHIENEIARIKKLIGGGGSWGESV

SEQ ID NO: 87 - protein, 39 aa, Artificial Sequence
  CC-Tet-GS4-HWLKV
GELAAIKQELAAIKKELAAIKWELAAIKQGGGGSHWLKV

SEQ ID NO: 88 - protein, 39 aa, Artificial Sequence
  CC-Tet-GS4-NSIRV
GELAAIKQELAAIKKELAAIKWELAAIKQGGGGSNSIRV

SEQ ID NO: 89 - protein, 39 aa, Artificial Sequence
  CC-Tet-GS4-IETDV
GELAAIKQELAAIKKELAAIKWELAAIKQGGGGSIETDV

SEQ ID NO: 90 - protein, 40 aa, Artificial Sequence
  CC-Tet-GS4-RRTTPV
GELAAIKQELAAIKKELAAIKWELAAIKQGGGGSRRTTPV

SEQ ID NO: 91 - protein, 40 aa, Artificial Sequence
  CC-Tet-GS4-YKQTSV
GELAAIKQELAAIKKELAAIKWELAAIKQGGGGSYKQTSV

SEQ ID NO: 92 - protein, 39 aa, Artificial Sequence
  CC-Tet-GS4-WGESV
GELAAIKQELAAIKKELAAIKWELAAIKQGGGGSWGESV

SEQ ID NO: 93 - protein, 39 aa, Artificial Sequence
  CC-Hex2-GS4-HWLKV
GEIAKSLKEIAKSLKEIAWSLKEIAKSLKGGGGSHWLKV

SEQ ID NO: 94 - protein, 39 aa, Artificial Sequence
  CC-Hex2-GS4-NSIRV
GEIAKSLKEIAKSLKEIAWSLKEIAKSLKGGGGSNSIRV

SEQ ID NO: 95 - protein, 39 aa, Artificial Sequence
  CC-Hex2-GS4-IETDV
GEIAKSLKEIAKSLKEIAWSLKEIAKSLKGGGGSIETDV

SEQ ID NO: 96 - protein, 40 aa, Artificial Sequence
  CC-Hex2-GS4-RRTTPV
GEIAKSLKEIAKSLKEIAWSLKEIAKSLKGGGGSRRTTPV

SEQ ID NO: 97 - protein, 40 aa, Artificial Sequence
  CC-Hex2-GS4-YKQTSV
GEIAKSLKEIAKSLKEIAWSLKEIAKSLKGGGGSYKQTSV

SEQ ID NO: 98 - protein, 39 aa, Artificial Sequence
  CC-Hex2-GS4-WGESV
GEIAKSLKEIAKSLKEIAWSLKEIAKSLKGGGGSWGESV

SEQ ID NO: 99 - protein, 40 aa, Artificial Sequence
  GCN4p1-GS4-HWLKV
RMKQLEDKVEELLSKNYHLENEVARLKKLVGGGGSHWLKV

SEQ ID NO: 100 - protein, 41 aa, Artificial Sequence
  GCN4p1-GS4-RRTTPV
RMKQLEDKVEELLSKNYHLENEVARLKKLVGGGGSRRTTPV

SEQ ID NO: 101 - protein, 41 aa, Artificial Sequence
  GCN4p1(7P14P)-GS4-RRTTPV
RMKQLEPKVEELLPKNYHLENEVARLKKLVGGGGSRRTTPV

SEQ ID NO: 102 - protein, 40 aa, Artificial Sequence
  GCN4p1-GS4-IETDV
RMKQLEDKVEELLSKNYHLENEVARLKKLVGGGGSIETDV

SEQ ID NO: 103 - protein, 40 aa, Artificial Sequence
  GCN4p1(7P14P)-GS4-IETDV
RMKQLEPKVEELLPKNYHLENEVARLKKLVGGGGSIETDV

SEQ ID NO: 104 - protein, 22 aa, Artificial Sequence
  Generic coiled coil
  MISC_FEATURE (2)..(7): any proteinogenic or non-proteinogenic amino acid residue.
  MISC_FEATURE (9)..(14): any proteinogenic or non-proteinogenic amino acid residue.
  MISC_FEATURE (16)..(21): any proteinogenic or non-proteinogenic amino acid residue.
LXXXXXXLXXXXXXLXXXXXXL

SEQ ID NO: 105 - protein, 29 aa, Artificial Sequence
  Generic trimeric coiled coil
  MISC_FEATURE (2)..(3): any proteinogenic or non-proteinogenic amino acid residue.
  MISC_FEATURE (5)..(7): any proteinogenic or non-proteinogenic amino acid residue.
  MISC_FEATURE (9)..(10): any proteinogenic or non-proteinogenic amino acid residue.
  MISC_FEATURE (12)..(14): any proteinogenic or non-proteinogenic amino acid residue.
  MISC_FEATURE (16)..(17): any proteinogenic or non-proteinogenic amino acid residue.
  MISC_FEATURE (19)..(21): any proteinogenic or non-proteinogenic amino acid residue.
  MISC_FEATURE (23)..(24): any proteinogenic or non-proteinogenic amino acid residue.
  MISC_FEATURE (26)..(28): any proteinogenic or non-proteinogenic amino acid residue.
MXXLXXXVXXLXXXQXXLXXXVXXLXXXV

SEQ ID NO: 106 - protein, 29 aa, Artificial Sequence
  Generic tetrameric coiled coil
  MISC_FEATURE (2)..(3): any proteinogenic or non-proteinogenic amino acid residue.
  MISC_FEATURE (5)..(7): any proteinogenic or non-proteinogenic amino acid residue.
  MISC_FEATURE (9)..(10): any proteinogenic or non-proteinogenic amino acid residue.
  MISC_FEATURE (12)..(14): any proteinogenic or non-proteinogenic amino acid residue.
  MISC_FEATURE (16)..(17): any proteinogenic or non-proteinogenic amino acid residue.
  MISC_FEATURE (19)..(21): any proteinogenic or non-proteinogenic amino acid residue.
  MISC_FEATURE (23)..(24): any proteinogenic or non-proteinogenic amino acid residue.
  MISC_FEATURE (26)..(28): any proteinogenic or non-proteinogenic amino acid residue.
IXXIXXXIXXIXXXIXXIXXXIXXIXXXI

SEQ ID NO: 107 - protein, 25 aa, Artificial Sequence
  Generic tetrameric coiled coil
  MISC_FEATURE (2)..(3): any proteinogenic or non-proteinogenic amino acid residue.
  MISC_FEATURE (5)..(7): any proteinogenic or non-proteinogenic amino acid residue.
  MISC_FEATURE (9)..(10): any proteinogenic or non-proteinogenic amino acid residue.
  MISC_FEATURE (12)..(14): any proteinogenic or non-proteinogenic amino acid residue.
  MISC_FEATURE (16)..(17): any proteinogenic or non-proteinogenic amino acid residue.
  MISC_FEATURE (19)..(21): any proteinogenic or non-proteinogenic amino acid residue.
  MISC_FEATURE (23)..(24): any proteinogenic or non-proteinogenic amino acid residue.
LXXIXXXLXXIXXXLXXIXXXLXXI

SEQ ID NO: 108 - protein, 26 aa, Artificial Sequence
  Generic hexameric coiled coil
  MISC_FEATURE (2)..(4): any proteinogenic or non-proteinogenic amino acid residue.
  MISC_FEATURE (6)..(7): any proteinogenic or non-proteinogenic amino acid residue.
  MISC_FEATURE (9)..(11): any proteinogenic or non-proteinogenic amino acid residue.
  MISC_FEATURE (13)..(14): any proteinogenic or non-proteinogenic amino acid residue.
  MISC_FEATURE (16)..(18): any proteinogenic or non-proteinogenic amino acid residue.
  MISC_FEATURE (20)..(21): any proteinogenic or non-proteinogenic amino acid residue.
  MISC_FEATURE (23)..(25): any proteinogenic or non-proteinogenic amino acid residue.
IXXXLXXIXXXLXXIXXXLXXIXXXL

SEQ ID NO: 109 - DNA, 93 bp, Artificial Sequence
  GCN4p1(NQ)
atgcgcatgaaacagctggaagataaagtggaagaactgctgagcaaacagtatcatctggaaaacgaagtggcgcgcctgaaaaaactggtg

SEQ ID NO: 110 - DNA, 93 bp, Artificial Sequence
  GCN4p1(LI)
atgcgcatcaaacagatcgaagataaaatcgaagaaatcctgagcaaaatctatcatatcgaaaacgaaatcgcgcgcatcaaaaaactgatc

SEQ ID NO: 111 - DNA, 87 bp, Artificial Sequence
  CC-Tet
ggcgagctggccgccatcaagcaggagctggccgccatcaagaaggagctggccgccatcaagtgggagctggccgccatcaagcag

SEQ ID NO: 112 - DNA, 87 bp, Artificial Sequence
  CC-Hex2
ggcgagatcgccaagagcctgaaggagatcgccaagagcctgaaggagatcgcctggagcctgaaggagatcgccaagagcctgaag

SEQ ID NO: 113 - DNA, 15 bp, Artificial Sequence
  HWLKV
cattggctgaaagtg

SEQ ID NO: 114 - DNA, 15 bp, Artificial Sequence
  NSIRV
aacagcatcagggtg

SEQ ID NO: 115 - DNA, 15 bp, Artificial Sequence
  IETDV
atcgagaccgacgtg

SEQ ID NO: 116 - DNA, 18 bp, Artificial Sequence
  RRTTPV
aggaggaccacccccgtg

SEQ ID NO: 117 - DNA, 18 bp, Artificial Sequence
  YKQTSV
tacaagcagaccagcgtg

SEQ ID NO: 118 - DNA, 15 bp, Artificial Sequence
  WGESV
tggggcgagagcgtg

SEQ ID NO: 119 - DNA, 15 bp, Artificial Sequence
  GS4
ggcggcggcggcagc

SEQ ID NO: 120 - DNA, 120 bp, Artificial Sequence
  GCN4p1(NQ)-GS4-HWLKV
cgcatgaaacagctggaagataaagtggaagaactgctgagcaaacagtatcatctggaaaacgaagtggcgcgcctgaaaaaactggtgggcggcggcggcagccattggctgaaagtg

SEQ ID NO: 121 - DNA, 120 bp, Artificial Sequence
  GCN4p1(NQ)-GS4-NSIRV
cgcatgaaacagctggaagataaagtggaagaactgctgagcaaacagtatcatctggaaaacgaagtggcgcgcctgaaaaaactggtgggcggcggcggcagcaacagcatcagggtg

SEQ ID NO: 122 - DNA, 120 bp, Artificial Sequence
  GCN4p1(NQ)-GS4-IETDV
cgcatgaaacagctggaagataaagtggaagaactgctgagcaaacagtatcatctggaaaacgaagtggcgcgcctgaaaaaactggtgggcggcggcggcagcatcgagaccgacgtg

SEQ ID NO: 123 - DNA, 123 bp, Artificial Sequence
  GCN4p1(NQ)-GS4-RRTTPV
cgcatgaaacagctggaagataaagtggaagaactgctgagcaaacagtatcatctggaaaacgaagtggcgcgcctgaaaaaactggtgggcggcggcggcagcaggaggaccacccccgtg

SEQ ID NO: 124 - DNA, 123 bp, Artificial Sequence
  GCN4p1(NQ)-GS4-YKQTSV
cgcatgaaacagctggaagataaagtggaagaactgctgagcaaacagtatcatctggaaaacgaagtggcgcgcctgaaaaaactggtgggcggcggcggcagctacaagcagaccagcgtg

SEQ ID NO: 125 - DNA, 120 bp, Artificial Sequence
  GCN4p1(NQ)-GS4-WGESV
cgcatgaaacagctggaagataaagtggaagaactgctgagcaaacagtatcatctggaaaacgaagtggcgcgcctgaaaaaactggtgggcggcggcggcagctggggcgagagcgtg

SEQ ID NO: 126 - DNA, 120 bp, Artificial Sequence
  GCN4p1(LI)-GS4-HWLKV
cgcatcaaacagatcgaagataaaatcgaagaaatcctgagcaaaatctatcatatcgaaaacgaaatcgcgcgcatcaaaaaactgatcggcggcggcggcagccattggctgaaagtg

SEQ ID NO: 127 - DNA, 120 bp, Artificial Sequence
  GCN4p1(LI)-GS4-NSIRV
cgcatcaaacagatcgaagataaaatcgaagaaatcctgagcaaaatctatcatatcgaaaacgaaatcgcgcgcatcaaaaaactgatcggcggcggcggcagcaacagcatcagggtg

SEQ ID NO: 128 - DNA, 120 bp, Artificial Sequence
  GCN4p1(LI)-GS4-IETDV
cgcatcaaacagatcgaagataaaatcgaagaaatcctgagcaaaatctatcatatcgaaaacgaaatcgcgcgcatcaaaaaactgatcggcggcggcggcagcatcgagaccgacgtg

SEQ ID NO: 129 - DNA, 123 bp, Artificial Sequence
  GCN4p1(LI)-GS4-RRTTPV
cgcatcaaacagatcgaagataaaatcgaagaaatcctgagcaaaatctatcatatcgaaaacgaaatcgcgcgcatcaaaaaactgatcggcggcggcggcagcaggaggaccacccccgtg

SEQ ID NO: 130 - DNA, 123 bp, Artificial Sequence
  GCN4p1(LI)-GS4-YKQTSV
cgcatcaaacagatcgaagataaaatcgaagaaatcctgagcaaaatctatcatatcgaaaacgaaatcgcgcgcatcaaaaaactgatcggcggcggcggcagctacaagcagaccagcgtg

SEQ ID NO: 131 - DNA, 120 bp, Artificial Sequence
  GCN4p1(LI)-GS4-WGESV
cgcatcaaacagatcgaagataaaatcgaagaaatcctgagcaaaatctatcatatcgaaaacgaaatcgcgcgcatcaaaaaactgatcggcggcggcggcagctggggcgagagcgtg

SEQ ID NO: 132 - DNA, 117 bp, Artificial Sequence
  CC-Tet-GS4-HWLKV
ggcgagctggccgccatcaagcaggagctggccgccatcaagaaggagctggccgccatcaagtgggagctggccgccatcaagcagggcggcggcggcagccactggctgaaggtg

SEQ ID NO: 133 - DNA, 117 bp, Artificial Sequence
  CC-Tet-GS4-NSIRV
ggcgagctggccgccatcaagcaggagctggccgccatcaagaaggagctggccgccatcaagtgggagctggccgccatcaagcagggcggcggcggcagcaacagcatcagggtg

SEQ ID NO: 134 - DNA, 117 bp, Artificial Sequence
  CC-Tet-GS4-IETDV
ggcgagctggccgccatcaagcaggagctggccgccatcaagaaggagctggccgccatcaagtgggagctggccgccatcaagcagggcggcggcggcagcatcgagaccgacgtg

SEQ ID NO: 135 - DNA, 120 bp, Artificial Sequence
  CC-Tet-GS4-RRTTPV
ggcgagctggccgccatcaagcaggagctggccgccatcaagaaggagctggccgccatcaagtgggagctggccgccatcaagcagggcggcggcggcagcaggaggaccacccccgtg

SEQ ID NO: 136 - DNA, 120 bp, Artificial Sequence
  CC-Tet-GS4-YKQTSV
ggcgagctggccgccatcaagcaggagctggccgccatcaagaaggagctggccgccatcaagtgggagctggccgccatcaagcagggcggcggcggcagctacaagcagaccagcgtg

SEQ ID NO: 137 - DNA, 117 bp, Artificial Sequence
  CC-Tet-GS4-WGESV
ggcgagctggccgccatcaagcaggagctggccgccatcaagaaggagctggccgccatcaagtgggagctggccgccatcaagcagggcggcggcggcagctggggcgagagcgtg

SEQ ID NO: 138 - DNA, 117 bp, Artificial Sequence
  CC-Hex2-GS4-HWLKV
ggcgagatcgccaagagcctgaaggagatcgccaagagcctgaaggagatcgcctggagcctgaaggagatcgccaagagcctgaagggcggcggcggcagccactggctgaaggtg

SEQ ID NO: 139 - DNA, 117 bp, Artificial Sequence
  CC-Hex2- GS4-NSIRV
ggcgagatcgccaagagcctgaaggagatcgccaagagcctgaaggagatcgcctggagcctgaaggagatcgccaagagcctgaagggcggcggcggcagcaacagcatcagggtg

SEQ ID NO: 140 - DNA, 117 bp, Artificial Sequence
  CC-Hex2-GS4-IETDV
ggcgagatcgccaagagcctgaaggagatcgccaagagcctgaaggagatcgcctggagcctgaaggagatcgccaagagcctgaagggcggcggcggcagcatcgagaccgacgtg

SEQ ID NO: 141 - DNA, 120 bp, Artificial Sequence
  CC-Hex2-GS4-RRTTPV
ggcgagatcgccaagagcctgaaggagatcgccaagagcctgaaggagatcgcctggagcctgaaggagatcgccaagagcctgaagggcggcggcggcagcaggaggaccacccccgtg

SEQ ID NO: 142 - DNA, 120 bp, Artificial Sequence
  CC-Hex2-GS4-YKQTSV
ggcgagatcgccaagagcctgaaggagatcgccaagagcctgaaggagatcgcctggagcctgaaggagatcgccaagagcctgaagggcggcggcggcagctacaagcagaccagcgtg

SEQ ID NO: 143 - DNA, 117 bp, Artificial Sequence
  CC-Hex2- GS4-WGESV
ggcgagatcgccaagagcctgaaggagatcgccaagagcctgaaggagatcgcctggagcctgaaggagatcgccaagagcctgaagggcggcggcggcagctggggcgagagcgtg

SEQ ID NO: 144 - protein, 40 aa, Artificial Sequence
  GCN4p1(LI)-GS4-NSVRV
RIKQIEDKIEEILSKIYHIENEIARIKKLIGGGGSNSVRV

SEQ ID NO: 145 - protein, 40 aa, Artificial Sequence
  GCN4p1-GS4-NSVRV
RMKQLEDKVEELLSKNYHLENEVARLKKLVGGGGSNSVRV

SEQ ID NO: 146 - protein, 39 aa, Artificial Sequence
  CC-Hex2-GS4-NSVRV
GEIAKSLKEIAKSLKEIAWSLKEIAKSLKGGGGSNSVRV

SEQ ID NO: 147 - protein, 30 aa, Artificial Sequence
  GCN4p1(ILI)
RMKQIEDKLEEILSKLYHIENELARIKKLL

SEQ ID NO: 148 - protein, 40 aa, Artificial Sequence
  GCN4p1(ILI)-GS4-NSVRV
RMKQIEDKLEEILSKLYHIENELARIKKLLGGGGSHWLKV

SEQ ID NO: 149 - protein, 40 aa, Artificial Sequence
  GCN4p1(ILI)-GS4-NSVRV
RMKQIEDKLEEILSKLYHIENELARIKKLLGGGGSNSVRV

SEQ ID NO: 150 - protein, 40 aa, Artificial Sequence
  GCN4p1(ILI)-GS4-IETDV
RMKQIEDKLEEILSKLYHIENELARIKKLLGGGGSIETDV

SEQ ID NO: 151 - protein, 41 aa, Artificial Sequence
  GCN4p1(ILI)-GS4-RRTTPV
RMKQIEDKLEEILSKLYHIENELARIKKLLGGGGSRRTTPV

SEQ ID NO: 152 - protein, 40 aa, Artificial Sequence
  GCN4p1(ILI)-GS4-WGESV
RMKQIEDKLEEILSKLYHIENELARIKKLLGGGGSWGESV

SEQ ID NO: 153 - protein, 40 aa, Artificial Sequence
  GCN4p1-GS4-GS4
RMKQLEDKVEELLSKNYHLENEVARLKKLVGGGGSGGGGS

SEQ ID NO: 154 - protein, 34 aa, Artificial Sequence
  ATF7-pII
VSSIEKKIEEITSQIIQISNEITLIRNEIAQIKQ

SEQ ID NO: 155 - protein, 52 aa, Artificial Sequence
  HA-GCN4p1(LI)-GS4-IETDV
MYPYDVPDYAGSRIKQIEDKIEEILSKIYHIENEIARIKKLIGGGGSIETDV

SEQ ID NO: 156 - protein, 52 aa, Artificial Sequence
  HA-GCN4p1(LI)-GS4-HWLKV
MYPYDVPDYAGSRIKQIEDKIEEILSKIYHIENEIARIKKLIGGGGSHWLKV

SEQ ID NO: 157 - protein, 52 aa, Artificial Sequence
  HA-GCN4p1(LI)-GS4-WGESV
MYPYDVPDYAGSRIKQIEDKIEEILSKIYHIENEIARIKKLIGGGGSWGESV

SEQ ID NO: 158 - protein, 52 aa, Artificial Sequence
  HA-GCN4p1-GS4-GS4
MYPYDVPDYAGSRMKQLEDKVEELLSKNYHLENEVARLKKLVGGGGSGGGGS

SEQ ID NO: 159 - protein, 34 aa, Artificial Sequence
  ATF2-pII
VQSIEKKIEDISSLIGQIQSEITLIRNEIAQIKQ

SEQ ID NO: 160 - protein, 39 aa, Artificial Sequence
  NRP-pII
DRAVIKEISEKIELIEKAIASIQLQIDEIKQTIAKIEED

SEQ ID NO: 161 - protein, 47 aa, Artificial Sequence
  PIX-pII
EEKSLVDTIYAIKDEIQEIRQDNKKIKKSIEEIQRAIKDLEKLIRKI

SEQ ID NO: 162 - protein, 33 aa, Artificial Sequence
  HLF-pII
EKEISAIRQEIADIRKEIGKIKNIIAKIEARHG

SEQ ID NO: 163 - protein, 33 aa, Artificial Sequence
  DBP-pII
EKEIALIRQEIVAIRQEISHIRAVISRIQAQHG

SEQ ID NO: 164 - protein, 31 aa, Artificial Sequence
  TEF-pII
EKEITAIRTEIAEIRKEIGKIKTIISKIETK

SEQ ID NO: 165 - protein, 42 aa, Artificial Sequence
  NRBI-pII
SPSELDISKISHKIKEIQIKIAVIEAEIQKIKTKIQAIENEK

SEQ ID NO: 166 - protein, 36 aa, Artificial Sequence
  CREB4-pII
ESRIAAISAQIQEIQKKIQEIERHIISIVAQIRQIQ

SEQ ID NO: 167 - protein, 36 aa, Artificial Sequence
  CREBH-pII
ETRISAITAQIQEIQRKILHIEKQILSILEQIKKIQ

SEQ ID NO: 168 - protein, 42 aa, Artificial Sequence
  MAT2-pII
EKHDQCKCENLIMFQNLANEEIRKITQRIEEITQRIEAIENR

SEQ ID NO: 169 - DNA, 135 bp, Artificial Sequence
  GCN4p1(LI)-GS4-NSVRV
cgcatcaaacagatcgaagataaaatcgaagaaatcctgagcaaaatctatcatatcgaaaacgaaatcgcgcgcatcaaaaaactgatcggcggcggcggcagcaacagcgtgagggtgaacagcgtgagggtg

SEQ ID NO: 170 - DNA, 123 bp, Artificial Sequence
  GCN4p1-GS4-NSVRV
agaatgaagcagctggaggacaaggtggaggagctgctgagcaagaactaccacctggagaacgaggtggccagactgaagaagctggtgggcggcggcggcagcatcaacagcgtgagggtg

SEQ ID NO: 171 - DNA, 120 bp, Artificial Sequence
  GCN4p1(NQ)-GS4-NSVRV
cgcatgaaacagctggaagataaagtggaagaactgctgagcaaacagtatcatctggaaaacgaagtggcgcgcctgaaaaaactggtgggcggcggcggcagcaacagcgtgagggtg

SEQ ID NO: 172 - DNA, 102 bp, Artificial Sequence
  CC-Hex2-GS4-NSVRV
ggcgagatcgccaagagcctgaaggagatcgccaagagcctgaaggagatcgcctggagcctgaaggagatcgccaagagcctgaagggcggcggcggcagc

SEQ ID NO: 173 - DNA, 90 bp, Artificial Sequence
  GCN4p1(ILI)
cgcatgaaacagatcgaagataaactggaagaaatcctgagcaaactgtatcatatcgaaaacgaactggcgcgcatcaaaaaactggtg

SEQ ID NO: 174 - DNA, 120 bp, Artificial Sequence
  GCN4p1(ILI)-GS4-HWLKV
cgcatgaaacagatcgaagataaactggaagaaatcctgagcaaactgtatcatatcgaaaacgaactggcgcgcatcaaaaaactggtgggcggcggcggcagccattggctgaaagtg

SEQ ID NO: 175 - DNA, 120 bp, Artificial Sequence
  GCN4p1(ILI)-GS4-NSVRV
cgcatgaaacagatcgaagataaactggaagaaatcctgagcaaactgtatcatatcgaaaacgaactggcgcgcatcaaaaaactggtgggcggcggcggcagcaacagcgtgagggtg

SEQ ID NO: 176 - DNA, 120 bp, Artificial Sequence
  GCN4p1(ILI)-GS4-IETDV
cgcatgaaacagatcgaagataaactggaagaaatcctgagcaaactgtatcatatcgaaaacgaactggcgcgcatcaaaaaactggtgggcggcggcggcagcatcgagaccgacgtg

SEQ ID NO: 177 - DNA, 123 bp, Artificial Sequence
  GCN4p1(ILI)-GS4-RRTTPV
cgcatgaaacagatcgaagataaactggaagaaatcctgagcaaactgtatcatatcgaaaacgaactggcgcgcatcaaaaaactggtgggcggcggcggcagcaggaggaccacccccgtg

SEQ ID NO: 178 - DNA, 120 bp, Artificial Sequence
  GCN4p1(ILI)-GS4-WGESV
cgcatgaaacagatcgaagataaactggaagaaatcctgagcaaactgtatcatatcgaaaacgaactggcgcgcatcaaaaaactggtgggcggcggcggcagctggggcgagagcgtg

SEQ ID NO: 179 - DNA, 120 bp, Artificial Sequence
  GCN4p1-GS4-GS4
cgcatgaaacagctggaagataaagtggaagaactgctgagcaaaaactatcatctggaaaacgaagtggcgcgcctgaaaaaactggtgggcggcggcggcagcggcggcggcggcagc

SEQ ID NO: 180 - DNA, 102 bp, Artificial Sequence
  ATF7-pII
gtgagcagcatcgagaagaagatcgaggagatcaccagccagatcatccagatcagcaacgagatcaccctgatcagaaacgagatcgcccagatcaagcag

SEQ ID NO: 181 - DNA, 15 bp, Artificial Sequence
  NSVRV
aacagcgtgagggtg

SEQ ID NO: 182 - DNA, 102 bp, Artificial Sequence
  ATF2-pII
gtgcagagcatcgagaagaagatcgaggacatcagcagcctgatcggccagatccagagcgagatcaccctgatcagaaacgagatcgcccagatcaagcag

SEQ ID NO: 183 - DNA, 117 bp, Artificial Sequence
  NRP-pII
gacagagccgtgatcaaggagatcagcgagaagatcgagctgatcgagaaggccatcgccagcatccagctgcagatcgacgagatcaagcagaccatcgccaagatcgaggaggac

SEQ ID NO: 184 - DNA, 141 bp, Artificial Sequence
  PIX-pII
gaggagaagagcctggtggacaccatctacgccatcaaggacgagatccaggagatcagacaggacaacaagaagatcaagaagagcatcgaggagatccagagagccatcaaggacctggagaagctgatcagaaagatc

SEQ ID NO: 185 - DNA, 99 bp, Artificial Sequence
  HLF-pII
gagaaggagatcagcgccatcagacaggagatcgccgacatcagaaaggagatcggcaagatcaagaacatcatcgccaagatcgaggccagacacggc

SEQ ID NO: 186 - DNA, 99 bp, Artificial Sequence
  DBP-pII
gagaaggagatcgccctgatcagacaggagatcgtggccatcagacaggagatcagccacatcagagccgtgatcagcagaatccaggcccagcacggc

SEQ ID NO: 187 - DNA, 93 bp, Artificial Sequence
  TEF-pII
gagaaggagatcaccgccatcagaaccgagatcgccgagatcagaaaggagatcggcaagatcaagaccatcatcagcaagatcgagaccaag

SEQ ID NO: 188 - DNA, 126 bp, Artificial Sequence
  NRBI-pII
agccccagcgagctggacatcagcaagatcagccacaagatcaaggagatccagatcaagatcgccgtgatcgaggccgagatccagaagatcaagaccaagatccaggccatcgagaacgagaag

SEQ ID NO: 189 - DNA, 108 bp, Artificial Sequence
  CREB4-pII
gagagcagaatcgccgccatcagcgcccagatccaggagatccagaagaagatccaggagatcgagagacacatcatcagcatcgtggcccagatcagacagatccag

SEQ ID NO: 190 - DNA, 108 bp, Artificial Sequence
  CREBH-pII
gagaccagaatcagcgccatcaccgcccagatccaggagatccagagaaagatcctgcacatcgagaagcagatcctgagcatcctggagcagatcaagaagatccag

SEQ ID NO: 191 - DNA, 126 bp, Artificial Sequence
  MAT2-pII
gagaagcacgaccagtgcaagtgcgagaacctgatcatgttccagaacctggccaacgaggagatcagaaagatcacccagagaatcgaggagatcacccagagaatcgaggccatcgagaacaga

SEQ ID NO: 192 - DNA, 132 bp, Artificial Sequence
  ATF7-GS4-HWLKV
gtgagcagcatcgagaagaagatcgaggagatcaccagccagatcatccagatcagcaacgagatcaccctgatcagaaacgagatcgcccagatcaagcagggcggcggcggcagccattggctgaaagtg

SEQ ID NO: 193 - DNA, 132 bp, Artificial Sequence
  ATF7-GS4-IETDV
gtgagcagcatcgagaagaagatcgaggagatcaccagccagatcatccagatcagcaacgagatcaccctgatcagaaacgagatcgcccagatcaagcagggcggcggcggcagcatcgagaccgacgtg

SEQ ID NO: 194 - protein, 44 aa, Artificial Sequence
  ATF7-GS4-HWLKV
VSSIEKKIEEITSQIIQISNEITLIRNEIAQIKQGGGGSHWLKV

SEQ ID NO: 195 - protein, 44 aa, Artificial Sequence
  ATF7-GS4-IETDV
VSSIEKKIEEITSQIIQISNEITLIRNEIAQIKQGGGGSIETDV

SEQ ID NO: 196 - protein, 6 aa, Artificial Sequence
  Generic Class I C5-C6 variants
  MISC_FEATURE (1)..(1): Y, R or is absent
  MISC_FEATURE (2)..(2): R, K, I or is absent
  MISC_FEATURE (3)..(3): T, E, Q; or is absent
  MISC_FEATURE (4)..(4): T
  MISC_FEATURE (5)..(5): D, S or P
  MISC_FEATURE (6)..(6): V
XXXXXX

(19) United States
(12) Patent Application Publication
(10) Pub. No.: US 2023/0348540 A1
(43) Pub. Date: Nov. 2, 2023

(54) Viral multimeric peptide constructs for targeting PDZ domains

(71) Applicant: University of Copenhagen, Copenhagen K (DK)

(72) Inventors: Andreas Toft Sørensen, Gentofte (DK); Kenneth Madsen, Virum (DK); Nikolaj Christensen, Allerød (DK); Kristian Strømgaard, Roskilde (DK)

(21) Appl. No.: 17/905,659

(22) PCT Filed: Mar. 5, 2021
(86) PCT No.: PCT/EP2021/055647

§ 371 Date: Sep. 6, 2022

(30) Foreign Application Priority Data
Mar. 6, 2020 (EP) 20161524.2

(51) Int. Cl. C07K 14/005; C12N 15/86; A61P 29/00

(52) U.S. Cl. CPC C07K 14/005; C12N 15/86; A61P 29/00; C12N 2750/14143

(57) ABSTRACT

The present invention relates to virally expressed peptides which bind to PDZ domains and thereby block PDZ domain mediated protein-protein interactions and expression vectors encoding these peptides. The virally expressed peptides comprise an oligomerization domain, capable of forming higher order constructs, such as trimers or tetramers, and a peptide ligand capable of binding to a PDZ domain. The invention furthermore relates to therapeutic use of said peptides and expression vectors encoding these peptides.

### TECHNICAL FIELD

The present invention relates to virally expressed peptides which bind to PDZ domains and thereby block PDZ domain mediated protein-protein interactions and to expression vectors coding for these peptides. The invention furthermore relates to therapeutic use of said peptides and expression vectors coding for these peptides.

### BACKGROUND

Synaptic plasticity serves as the molecular substrate for learning and memory. In the glutamatergic synapse release of glutamate activates in particular the N-methyl-D-aspartate receptors (NMDARs) and the α-amino-3-hydroxy-5-methyl-4-isoxazole-propionic acid receptors (AMPARs), both ligand-gated ion-channels. Activation of these receptors allows for an influx of Na⁺ in AMPARs and Ca²⁺ in the case of NMDARs. In diseased states, such as ischemia after stroke, neuropathic pain and addiction, abnormal synaptic stimulation and transmission cause maladaptive plasticity leading to hyper-sensitization of glutamatergic synapses in part through expression of calcium permeable (CP) AMPA-type glutamate receptors (CP-AMPARs).

Numerous diseased states, including ischemia after stroke and head injury, amyotrophic lateral sclerosis (ALS), epilepsy, Alzheimer's disease, neuropathic pain, hearing disorders (e.g. tinnitus) and addiction, involve an over-activation or sensitization of the glutamate system, yet the NMDA receptor antagonists such as ketamine (anaesthetic) are currently the only drugs in clinical use that target the glutamate system. Diseases such as neuropathic pain, excitotoxicity following ischemia and drug addiction are currently without any effective therapy. There is thus a need for new methods for targeting the glutamate system to allow treatment of such diseases.

Protein-protein interactions (PPIs) are vital for most biochemical and cellular processes and are often mediated by scaffold and signal transduction complexes. One of the most abundant classes of human facilitators of PPIs is the family of postsynaptic density protein-95 (PSD-95)/Discs-large/ZO-1 (PDZ) domains. PDZ domains are known to increase the specificity and efficiency of intracellular communication networks downstream of receptor activation by facilitating several protein-protein interactions (PPIs). PDZ domains may be found in multidomain scaffold and anchoring proteins involved in trafficking, recruiting, and assembling of intracellular enzymes and membrane receptors into signal-transduction complexes. PDZ domain-containing proteins are involved in numerous signalling pathways, and are as a consequence associated with a range of diseases and disorders.

PDZ domain containing proteins, such as Protein Interacting with C Kinase-1 (PICK1) and Post synaptic density protein 95 (PSD-95), dynamically regulate the surface expression and activity of the glutamate receptors and therefore represent attractive alternate drug targets for treatment of diseases or disorders associated with maladaptive plasticity. Targeting of and inhibition of protein-protein interactions has, however, proven challenging due to a lack of sufficient potency of small molecule inhibitors, and the generally poor pharmacokinetic profiles of peptide drugs.

PICK1 is a PDZ domain containing scaffolding protein that plays a central role in synaptic plasticity. PICK1 is a functional dimer, with two PDZ domains flanking the central membrane binding BAR domain, which also mediates the dimerization. This protein is especially relevant for regulation of protein trafficking and cell migration by mediating and facilitating PPIs via its two PDZ domains. For example, the PICK1 PDZ domain interacts directly with the C-terminus of the GluA2 subunit of the AMPA receptors (AMPAR) as well as protein kinase A and C, thereby regulating AMPAR phosphorylation and surface expression and in turn synaptic plasticity tuning the efficacy of individual synapses.

PSD-95 is one of the major scaffolding proteins in the excitatory synapse and is expressed exclusively in the brain, with the highest content in the cortex and hippocampus. PSD-95 regulates the trafficking and localization of glutamate receptors such as AMPA-type or NMDA-type-receptors. PSD-95 comprises three PDZ domains located sequentially in the N-terminal end of the protein.

As described above, there is a high need for providing potent inhibitors of PDZ domains for treatment of diseases or disorders associated with maladaptive plasticity.

### SUMMARY

The present invention provides a polynucleotide encoding a high affinity peptide inhibitor towards PDZ domain containing proteins, such as for example protein interacting C kinase-1 (PICK1) or postsynaptic density protein 95 (PSD-95). The high affinity peptide inhibitor encoded by the polynucleotide of the present disclosure comprises a peptide ligand capable of binding to a PDZ domain and a further peptide part functioning as an oligomerization domain. Peptide ligands capable of binding to a PDZ domain are typically derived from the three to six C-terminal amino acid residues of an endogenous PDZ ligand and typically consist of or comprise a PDZ domain binding motif (PBM). The inventors have surprisingly found that by conjugation of a peptide ligand, which is capable of binding to PDZ domains, to a further peptide part functioning as an oligomerization domain, higher order constructs or structures, such as trimers or tetramers, are formed which possess markedly increased potency for targeting PDZ domain containing proteins, as compared to the peptide ligand itself or to a dimeric construct of the peptide ligand (Examples 4 and 5 and 8). Such high increase in potency could not be foreseen as a result of the oligomerization.

The polynucleotide of the present disclosure may be administered by viral delivery to provide gene therapy. The polynucleotide of the present disclosure may comprise a neuron-specific promotor, to provide expression of the polypeptide encoded by the polynucleotide selectively in neurons. The polynucleotide thus differs from existing compounds targeting PDZ domains in that it can be delivered with high efficacy and selectivity as a single viral injection thus lifting therapeutic outcome and patient compliance in patients with conditions such as neuropathic pain, excitotoxicity following ischemia or drug addiction, while reducing possible side effects. The polynucleotide of the present disclosure further differs from current glutamate receptor drugs by targeting the scaffolding proteins responsible for the trafficking of the receptor, rather than targeting the receptor directly. As demonstrated in the present disclosure, the polynucleotide of the present disclosure provides prophylaxis and/or treatment of a disease and/or disorder associated with maladaptive plasticity, such as provides treatment of inflammatory pain as demonstrated in example 10.

In a first aspect, the present disclosure provides a polynucleotide comprising a sequence encoding a polypeptide comprising:
a) a first polypeptide part comprising or consisting of an amino acid sequence capable of forming a trimer, tetramer and/or higher order oligomer; and
b) a second polypeptide part comprising or consisting of an amino acid sequence selected from the group consisting of Class I PDZ domains binding motifs (PBM), Class II PBM and Class III PBM,
   wherein the first and the second polypeptides are optionally operably linked via a linker.

In another aspect, the present disclosure provides a polynucleotide comprising a sequence encoding a polypeptide comprising:
a) a first polypeptide part comprising or consisting of an amino acid sequence selected from the group consisting of GCN4p1(NQ) (SEQ ID NO: 67), GCN4p1(LI) (SEQ ID NO: 68), GCN4p1(ILI) (SEQ ID NO: 147), CC-Tet (SEQ ID NO: 69), cc-Hex2 (SEQ ID NO: 70), and ATF7-pII (SEQ ID NO: 154); and
b) a second polypeptide part comprising or consisting of an amino acid sequence selected from the group consisting of Class I PDZ domains binding motifs (PBM), Class II PBM and Class III PBM,
   wherein the first and the second polypeptides are operably linked, optionally via a linker.

In a second aspect, the present disclosure provides an expression vector comprising the polynucleotide as disclosed herein.

In a further aspect, the present disclosure provides a polypeptide as disclosed herein.

In a further aspect, the present disclosure provides a host cell comprising the polynucleotide, the expression vector or polypeptide as disclosed herein.

In a further aspect, the present disclosure provides a pharmaceutical composition comprising the polynucleotide, the expression vector or polypeptide as disclosed herein.

In a further aspect, the polynucleotide, the expression vector, the polypeptide, the cell, and/or the pharmaceutical composition as disclosed herein is provided for use as a medicament.

In a further aspect, the polynucleotide, the expression vector, the polypeptide, the cell, and/or the pharmaceutical composition as disclosed herein is provided for use in the prophylaxis and/or treatment of a disease and/or disorder associated with maladaptive plasticity.

In a further aspect, a method of treatment or prevention of a disease and/or disorder associated with maladaptive plasticity is provided, the method comprising administering a therapeutically effective amount of the polynucleotide, the expression vector, the polypeptide, the cell, and/or the pharmaceutical composition in a subject in need thereof.

### DESCRIPTION OF DRAWINGS

FIG. 1: Oligomeric state and secondary structure of GCN4p1 variants. (A) Size exclusion chromatography of GCN4p1 variants with HWLKV (Class II) motif. (B) Size exclusion chromatography of GCN4p1 variants with RRTTPV (Class I) motif. (C) Circular dichroism of GCN4p1 variants with HWLKV (Class II) motif. (D) Circular dichroism of GCN4p1 variants with RRTTPV (Class I) motif.

FIG. 2: Fluorescence polarization competition binding curves for the unlabelled peptides. A fixed concentration of PICK1 (0.19 μM) and tracer 5-FAM-NPEG₄-(HWLKV)₂ (10 nM) was titrated with increasing concentration of the unlabelled peptides (HWLKV, Dimeric GCN4p1-HWLKV, GCN4p1(NQ)-HWLKV, or GCN4p1(LI)-HWLKV). This caused a displacement of the fluorescently labelled molecule (tracer) with the unlabelled peptides, and gave rise to decrease in the polarization value (mP) as seen in the plot. Data expressed as mean±SEM (n=3).

FIG. 3: Size exclusion chromatography of 40 μM PICK1 in absence or presence of μM dimeric GCN4p1-HWLKV and GCN4p1(NQ)-HWLKV (A) or dimeric GCN4p1-HWLKV and GCN4p1(LI)-HWLKV (B).

FIG. 4: Fluorescence polarization competition binding curves for the unlabelled peptides comprising (A) RRTTPV (Monomeric GCN4p1(7P14P)-RRTTPV, Dimeric GCN4p1-RRTTPV or GCN4p1(LI)-RRTTPV) or (B) IETDV (Monomeric GCN4p1(7P14P)-IETDV, Dimeric GCN4p1-IETDV or GCN4p1(LI)-IETDV). A fixed concentration of PSD-95 (0.15 μM) and tracer 5-FAM-NPEG₄-(IETAV)₂ (5 nM) was titrated with increasing concentration of the unlabelled peptides. This caused a displacement of the fluorescently labelled molecule (tracer) with the unlabelled peptides, and gave rise to decrease in the polarization value (mP) as seen in the plot. Data expressed as mean±SEM (n=3).

FIG. 5: SDS-PAGE sedimentation (A) and quantification (B) of 3 μM PSD-95 in absence (0 μM) or presence of 12 μM or 36 μM peptide comprising RRTTPV (SNTANRRTTPV (Stg), Dimeric GCN4p1-RRTTPV (dim-Stg) or GCN4p1(LI)-RRTTPV (tet-Stg)). S denotes the supernatant and P denotes the pellet fractions. (C) Fluorescence confocal microscopy of Alexa488-labeled PSD-95 bound to unlabelled peptides (Monomeric GCN4p1(7P14P)-RRTTPV, Dimeric GCN4p1-RRTTPV or GNC4p1(LI)-RRTTPV). (D) Size exclusion chromatography of PSD-95 in the absence 0 μM or presence of increasing amounts of Dimeric GCN4p1-RRTTPV or GCN4p1(LI)-RRTTPV. The decreasing maximal peak height, is indicating formation of complexes too large to enter the elution column.

FIG. 6: Effect of single amino acid substitutions in DAT C5 (HWLKV) on binding affinity. A library of 95 HWLKV peptides with single amino acids substitutions in position X1-X5 of the sequence HWLKV was tested in fluorescence polarization binding in competition with fluorescently labelled HWLKV. Data are given as fold change compared to the reference peptide HWLKV (set to 1) with darker shades indicating increase in affinity (up to 3-fold) and lighter shades indicating reduces affinity. White indicate disruption of binding and crosses indicate insoluble peptides. Peptides shown with % were not soluble in buffer and were dissolved in 10% DMSO.

FIG. 7: Fold affinity change measured using FP competition of a combinatorial peptide library combining single amino acid substitutions from previous single substitution screen. Screen suggests NSVRV/TSIRV as optimal 5-mer sequences, EIRV/YIIV as optimal 4-mer sequences, IIV/IRV as optimal 3-mer sequences. These sequences could not have been predicted from initial 5-mer sequence, HWLKV. x indicates insoluble or non-binding peptides.

FIG. 8: SEC-MALS experiments ratify oligomeric states of HWLKV peptide variants. MALS data suggests a; A, dimeric configuration of GCN4p1-GS4-HWLKV, B-D, trimeric configuration of GCN4p1(NQ)-GS4-HWLKV, GCN4p1(LI)-GS4-HWLKV and CC-tet-GS4-HWLKV, E, tetrameric configuration of GCN4p1(ILI)-GS4-HWLKV, F, hexameric configuration of CC-hex-GS4-HWLKV. A monomeric peptide is ˜4.5 kDa. CC-tet-GS4-HWLKV was expected to be in a parallel tetrameric configuration from prior art.

FIG. 9: Flow induced dispersion analysis (FIDA) suggests larger hydrodynamic radius of GCN4p1(LI)-GS4-IETDV compared to GCN4p1-GS4-IETDV, suggesting a larger oligomeric state of GCN4p1(LI)-GS4-IETDV than the dimeric state of GCN4p1-GS4-IETDV.

FIG. 10: Circular dichroism spectra validates helical structure of; A GCN4p1(ILI)-GS4-HWLKV; B CC-tet-GS4-HWLKV; C CC-Hex2-GS4-HWLKV; D GCN4p1(LI)-GS4-NSVRV; E GCN4p1(ILI)-GS4-NSVRV. Helical structure is estimated from the shape of the spectra.

FIG. 11: Circular dichroism spectra validates helical structure of; A GCN4p1(LI)-GS4-IETDV; B GCN4p1(ILI)-GS4-IETDV; C GCN4p1(LI)-GS4-YKQTSV; D GCN4p1(ILI)-GS4-RRTTPV; E CC-Hex2-GS4-RRTTPV. Helical structure is estimated from the shape of the spectra.

FIG. 12: SEC FPLC elution profile of 30 μM PICK1 in absence (black) or presence (grey) of; A 50 μM GCN4p1(LI)-GS4-HWLKV, B 50 μM GCN4p1(ILI)-GS4-HWLKV, or C, 50 μM CC-tet2-GS4-HWLKV. Data indicates formation of higher order oligomeric species of PICK1 when in complex with higher order oligomeric peptides.

FIG. 13: SEC FPLC elution profile of 30 μM PICK1 in absence (black) or presence (grey) of; A 50 μM GCN4p1(LI)-GS4-NSVRV, B 50 μM GCN4p1(ILI)-GS4-NSVRV. Data indicates formation of higher order oligomeric species of PICK1 when in complex with higher order oligomeric peptides.

FIG. 14: SEC FPLC elution profile of 10 μM FL-PSD-95 in absence (dark grey) or presence (light grey) of; A, 50 μM GCN4p1-GS4-IETDV, B 50 μM GCN4p1(LI)-GS4-IETDV, C 50 μM GCN4p1(ILI)-GS4-IETDV, or D, 50 μM CC-hex2-GS4-IETDV. Data indicates formation of higher order oligomeric species of PSD-95 when in complex with higher order oligomeric peptides.

FIG. 15: SEC FPLC elution profile of 10 μM FL-PSD-95 in absence (dark grey) or presence (light grey) of; A, 50 μM GCN4p1(LI)-GS4-RRTTPV, B, 50 μM GCN4p1(ILI)-GS4-RRTTPV, or C, 50 μM CC-Hex2-GS4-RRTTPV. Data indicates formation liquid-liquid phase separation of PSD-95 when in complex with higher order oligomeric peptides, seen as the drop maximal peak height.

FIG. 16: Confocal microscopy validation of LLPS formation for 100 μM PSD-95 PDZ1-2 in complex with; A 512 μM GCN4p1-GS4-RRTTPV; B 256 μM GCN4p1(LI)-GS4-RRTTPV; C 128 μM GCN4p1(ILI)-GS4-RRTTPV; D 128 μM CC-Hex2-GS4-RRTTPV. Images suggest that higher order oligomeric ligands enhance LLPS formation of PSD-95 PDZ1-2, at a lower threshold than for dimeric GCN4p1-GS4-RRTTPV. Scale bar indicates 10 μm.

FIG. 17: Fluorescence polarization competition binding curves for the unlabelled peptides. A fixed concentration of PICK1 (0.25 μM) and tracer 5-FAM-(HWLKV)₂ (10 nM) was titrated with increasing concentration of the unlabelled peptides. This caused a displacement of the fluorescently labelled molecule (tracer) with the unlabelled peptides, and gave rise to decrease in the polarization value (mP) as seen in the plot. Data expressed as mean±SEM (n=3).

FIG. 18: Fluorescence polarization competition binding curves for the unlabelled peptides. A fixed concentration of PSD-95 PDZ12 (0.15 μM) and tracer 5-FAM-(IETAV)₂ (5 nM) was titrated with increasing concentration of the unlabelled peptides. This caused a displacement of the fluorescently labelled molecule (tracer) with the unlabelled peptides, and gave rise to decrease in the polarization value (mP) as seen in the plot. Data expressed as mean±SEM (n=3).

FIG. 19: A) Pull-down experiment with PICK1 binding peptides (three left lanes) confirms target engagement with PICK1 protein, whereas the control peptide (biotin-Ahx-GCN4p1-GS-GS) does not bind PICK1. Input (lysate) lane is shown on the very right with two adjacent empty lanes.
B) Pull-down experiment with PSD-95 binding peptides (three left lanes) confirms target engagement with PSD-95 protein, whereas the control peptide (biotin-Ahx-GCN4p1-GS-GS) does not bind PSD-95 protein. The input lane is shown on the right.
C) Pull-down experiment with nNOS binding peptides (three left lanes) confirms target engagement with nNOS protein, whereas the control peptide (biotin-Ahx-GCN4p1-GS-GS) does not bind nNOS protein. The input lane is shown on the right.
D) Pull-down experiment confirming high degree of selective target engagement specified by their PDZ binding motif (HWLKV, PICK1; IETDV, PSD-95; WGESV; nNOS) respectively. The control peptide (biotin-Ahx-GCN4p1-GS-GS) does not bind any of the PDZ proteins (right lane). The input lane is shown on the left.

FIG. 20A:Intrathecal injection of rAAV2.8-hSyn-HA-GCN4p1(LI)-HWLKV-WPREpA relieves pain. Paw withdrawal threshold was measured in the CFA model of inflammatory pain. Data were analysed by a two-way RM ANOVA (Interaction: F(4, 40)=5,383, p=0.0015; Time: F(4, 40)=21.94, p<0,0001; Treatment: F(1, 10)=45.66, p<0,0001). Posthoc analysis (Šidák's multiple comparisons test) revealed significant pain relief at the injured paw (ipsilateral) at day 2 and day 4 post CFA injection (****p<0.0001) when comparing the active (AAV2.8-hSyn-HA-GCN4p1(LI)-GS4-HWLKV-WPREpA, n=6) against the non-binding control (rAAV2.8-hSyn-GCN4p1-GS4-GS4-WPREpA; n=6) treatment. BL=baseline Von Frey measurements.

FIG. 20B: Intrathecal injection of rAAV2.8-hSyn-HA-GCN4p1(LI)-IETDV-WPREpA relieves pain. Paw withdrawal threshold was measured in the CFA model of inflammatory pain. Data were analysed by a two-way RM ANOVA (Interaction: F(4, 56)=3,193, p=0.0197; Time: F(4, 56)=53.81, p<0,0001; Treatment: F(1, 14)=3,572, p=0.0796). Posthoc analysis (Šidák's multiple comparisons test) revealed significant pain relief at the injured paw (ipsilateral) at day 4 post CFA injection (*p<0.05) when comparing the active (AAV2.8-hSyn-HA-GCN4p1(LI)-GS4-IETDV-WPREpA, n=10) against the non-binding control (rAAV2.8-hSyn-GCN4p1-GS4-GS4-WPREpA; n=6) treatment. BL=baseline Von Frey measurements.

FIG. 20C: Intrathecal injection of rAAV2.8-hSyn-HA-GCN4p1(LI)-WGESV-WPREpA reliefs pain. Paw withdrawal threshold was measured in the CFA model of inflammatory pain. Data were analysed by a two-way RM ANOVA (Interaction: F(4, 56)=2,038, p=0.1014; Time: F(4, 56)=30.46, p<0,0001; Treatment: F(1, 14)=4,589, p=0.0502). Posthoc analysis (Šidák's multiple comparisons test) revealed significant pain relief at the injured paw (ipsilateral) at day 2 post CFA injection (*p<0.05) when comparing the active (AAV2.8-hSyn-HA-GCN4p1(LI)-GS4-WGESV-WPREpA, n=10) against the non-binding control (rAAV2.8-hSyn-GCN4p1-GS4-GS4-WPREpA; n=6) treatment. BL=baseline Von Frey measurements.

### DETAILED DESCRIPTION

### Definitions

PDZ domain binding motif (PBM) as used herein refers to a peptide ligand which is capable of binding to a PDZ domain. PBMs may be divided into three groups, Class I, II, and III PBMs, each having a characteristic three amino acid sequence. PDZ domains of different proteins show different selectivity towards Class I, II or III PBMs.

Amino acids, that are proteinogenic are named herein using either its 1-letter or 3-letter code according to the recommendations from IUPAC, see for example http://www.chem.qmw.ac.uk/iupac. If nothing else is specified an amino acid may be of D or L-form. In the description a 3-letter code starting with a capital letter indicates an amino acid of L-form, whereas a 3-letter code in small letters indicates an amino acid of D-form. In a preferred embodiment, the amino acids of the present disclosure are L-amino acids.

Hydrophobic amino acids, are amino acids having a hydrophobic side chain, examples of hydrophobic amino acids include alanine, isoleucine, leucine, methionine, phenylalanine, valine, proline and glycine.

AAV, adeno associated virus.

AAV1, Adeno-associated virus vectors serotype 1.

AAV2, Adeno-associated virus vectors serotype 2.

AAV5, Adeno-associated virus vectors serotype 5;

AAV8, Adeno-associated virus vectors serotype 8.

AAV9, Adeno-associated virus vectors serotype 9; PDZ, acronym combining the first letters of the first three proteins discovered to share the domain Postsynaptic density protein-95 (PSD-95), Drosophila homologue discs large tumor suppressor (DIgA) and Zonula occludens-1 protein (zo-1). PDZ domains are common structural domains of 80-90 amino-acids found in PDZ domain containing proteins, such as signalling proteins. Proteins containing PDZ domains often play a key role in anchoring receptor proteins in the membrane to cytoskeletal components. GS, glycine serine linker. GSx as used herein refers to a glycine linker having the sequence (G)ₓS, wherein X refers to the number of glycine residues in the linker. As an example, a GS4 linker comprises four glycine residues and has the sequence GGGGS.

hSyn, Human synapsin 1 gene promoter confers highly neuron-specific long-term transgene expression from a viral vector.

WPRE, Woodchuck Hepatitis Virus (WHP) Posttranscriptional Regulatory Element. Is a DNA sequence that, when transcribed creates a tertiary structure enhancing expression and is commonly used in molecular biology to increase expression of genes delivered by viral vectors.

Proteinogenic as used herein refers to the 20 amino acids that are encoded by the genetic code and constitute naturally occurring.

Non-proteinogenic amino acids are amino acids which are not used in nature as building blocks for protein biosynthesis and are thereby to be clearly delineated from the 20 proteinogenic amino acids.

The term ‘absent’ as used herein, e.g. “X₁ is H, L, I, A or is absent” is to be understood that the amino acid is not part of the sequence and that the residues directly adjacent to the absent amino acid are directly linked to each other by a conventional amide bond.

Amide bond is formed by a reaction between a carboxylic acid and an amine with concomitant elimination of water. Where the reaction is between two amino acid residues, the bond formed as a result of the reaction is known as a peptide linkage (peptide bond).

The term ‘operably linked’ as used herein indicates that the polynucleotide sequence encoding one or more polypeptides of interest and transcriptional regulatory sequences are connected in such a way as to permit expression of the polynucleotide sequence when introduced into a cell. Two polypeptide parts are considered operably linked when they form part of one polypeptide chain and each polypeptide part can perform its function.

The term “polypeptide” as used herein refers to a molecule comprising at least two amino acids. The amino acids may be natural or synthetic.

The term ‘disorder’ used herein refers to a disease or medical condition, and is an abnormal condition of an organism that impairs bodily functions, associated with specific symptoms and signs.

The term ‘polynucleotide’ used herein refers to a molecule which is an organic polymer molecule composed of nucleotide monomers covalently bonded in a chain. A “polynucleotide” as used herein refers to a molecule comprising at least two nucleic acids. The nucleic acids may be naturally occurring or modified. In a cellular setting the polynucleotide may be transcribed and translated to provide expression of the polypeptide encoded by the polynucleotide.

The term ‘promoter’ used herein refers to a region of DNA that facilitates the transcription of a particular gene. Promoters are typically located near the genes they regulate, on the same strand and upstream.

The term ‘medicament’ refers to any therapeutic or prophylactic agent which may be used in the treatment of a malady, affliction, condition, disease or injury in a patient. The NMDA receptor refers to the N-methyl-D-aspartate receptor (also known as the NMDA receptor or NMDAR) and is a glutamate receptor and ion channel protein found in nerve cells. The NMDA receptor is one of three types of ionotropic glutamate receptors.

The AMPA receptor refers to the α-amino-3-hydroxy-5-methyl-4-isoxazole-propionic acid (also known as the AMPA receptor or AMPAR) and is a glutamate receptor and ion channel protein found in nerve cells. The NMDA receptor is one of three types of ionotropic glutamate receptors.

The myc tag as used herein refers to a polypeptide derived from the c-myc gene product which can be added to a peptide or protein using recombinant DNA technology. It may be used for affinity chromatography for purification. A myc tag may be used for detection, isolation, and/or purification of the peptide or protein of interest.

The HA-tag as used herein refers to amino acids 98-106 of the Human influenza hemagglutinin (HA). It may be used as a general epitope tag in expression vectors. The HA-tag may facilitate the detection, isolation, and/or purification of the peptide or protein of interest.

The His-tag as used herein refers to a polyhistidine-tag comprising at least six histidine residues. The His-tag may be used for detection, isolation, and/or purification of the peptide or protein of interest.

Structure of PDZ Domain Inhibitors

The polynucleotide of the present disclosure encodes a PDZ domain inhibitor which comprises an oligomerization domain and a peptide ligand capable of binding to a PDZ domain. The oligomerization domain may be capable of self-assembling into homotrimers, homotetramers or higher order constructs. Self-assembly of the oligomerization domain results in higher order constructs comprising three, four or more peptide ligands capable of binding PDZ domains. These constructs are capable of inhibiting PDZ domain containing proteins and may provide treatment of diseases or disorders associated with maladaptive plasticity.

Thus, in one embodiment, a polynucleotide is provided comprising a sequence encoding a polypeptide comprising:
a) a first polypeptide part comprising or consisting of an amino acid sequence capable of forming a trimer, tetramer and/or higher order oligomer; and
b) a second polypeptide part comprising or consisting of an amino acid sequence selected from the group consisting of Class I PDZ domains binding motifs (PBM), Class II PBM and Class III PBM,
wherein the first and the second polypeptides are optionally operably linked via a linker.

In one embodiment, a polynucleotide is provided comprising a sequence encoding a polypeptide comprising:
a) a first polypeptide part comprising or consisting of an amino acid sequence capable of forming a trimer, tetramer and/or higher order oligomer; and
b) a second polypeptide part comprising or consisting of an amino acid sequence selected from the group consisting of Class I PDZ domains binding motifs (PBM), Class II PBM and Class III PBM, wherein the first and the second polypeptides are operably linked, optionally via a linker.

In one embodiment, a polynucleotide is provided comprising a sequence encoding a polypeptide comprising:
a) a first polypeptide part comprising or consisting of an amino acid sequence selected from the group consisting of GCN4p1(NQ) (SEQ ID NO: 67), GCN4p1(LI) (SEQ ID NO: 68), GCN4p1(ILI) (SEQ ID NO: 147), CC-Tet (SEQ ID NO: 69), cc-Hex2 (SEQ ID NO: 70), and ATF7-pII (SEQ ID NO: 154); and
b) a second polypeptide part comprising or consisting of an amino acid sequence selected from the group consisting of Class I PDZ domains binding motifs (PBM), Class II PBM and Class III PBM,
wherein the first and the second polypeptides are operably linked, optionally via a linker.

In one embodiment, a polypeptide encoded by the polynucleotide of the present disclosure is provided.

First Polypeptide Part

In one embodiment, the first polypeptide part of the present disclosure is an oligomerization domain. Said oligomerization domain may be capable of forming a trimer, a tetramer, a pentamer, a hexamer, a heptamer, and/or higher order constructs. In one embodiment, the first polypeptide part is capable of forming a homotrimer, a homotetramer, a homopentamer, a homohexamer, a homoheptamer, and/or higher order constructs.

In one embodiment, the number of polypeptides of the present disclosure associating to form an oligomer is equal to or greater than 3, such as equal to or greater than 4, for example equal to or greater than 5. In one embodiment, at least 3 polypeptides of the present disclosure associate to form an oligomer, such as at least 4 polypeptides, for example at least 5 polypeptide associate to form an oligomer.

In one embodiment, the number of polypeptides associating to form a oligomer is in the range of 3 to 7, such as in the range of 3 to 6, for example in the range of 3 to 5, such as in the range of 3 to 4.

In one embodiment, the oligomeric state of the polypeptide of the present disclosure is higher than 2. An oligomeric state higher than 2 may be confirmed by comparing the peptide in question having an oligomeric state higher than 2, to a given peptide known to form a dimer of approximately the same molecular weight, such as comparing with a polypeptide comprising GCN4p1, for example by using Flow induced dispersion analysis (FIDA), as demonstrated in Example 7 of the present disclosure.

As an example, the term “capable of forming a trimer” refers to the ability of the first polypeptide part of the present disclosure to interact with two identical first polypeptide parts of the present disclosure and form e.g. a trimer, such as a homotrimer. Such trimer may for instance be observed by analysis of the polypeptide by size exclusion chromatography (SEC), such as by the SEC method as described in Examples 3 of the present disclosure. Alternatively, the oligomeric state may be determined by Size exclusion chromatography Multi angle light scattering (SEC-MALS) as demonstrated in Example 7 of the present disclosure. Thus, the polynucleotide of the present disclosure may provide a monomeric polypeptide upon expression, which is capable of interacting with further polypeptide of the present disclosure to form trimer, tetramers and/or higher order constructs. The interaction of the three or more polypeptides may be facilitated via interaction of the first polypeptide parts having an alpha helical secondary structure, such as an amphipathic helix. Such interaction between three or more alpha helical first polypeptide parts may form a coiled coil interaction. In one embodiment, the first polypeptide parts of the three or more polypeptides capable of forming a trimer, tetramer and/or higher order constructs has a high alpha helical content, such as determined by circular dichroism. The interaction between monomers to form trimers, tetramers and/or higher order constructs may be facilitated by electrostatic interactions, such as hydrophobic interactions, salt-bridges and/or hydrogen bonding. In one embodiment, oligomerization of the first polypeptide part takes place in solution at physiologically relevant concentrations, both in vitro and in vivo.

In one embodiment, the first polypeptide part is an alpha helix, such as an amphipathic helix.

In one embodiment, the first polypeptide part is capable of forming a coiled coil, such as a coiled coil comprising three polypeptides, for example comprising four polypeptides, such as comprising five polypeptides, for example comprising six polypeptides, such as comprising seven polypeptides.

The first polypeptide part may comprise an amino acid sequence of the general formula LXXXXXXLXXXXXXLXXXXXXL (SEQ ID NO: 104),
wherein X is individually selected from any proteinogenic or non-proteinogenic amino acid residue. Such general formula represents a typical sequence of polypeptides which are capable of forming coiled coils.

The first polypeptide part may comprise an amino acid sequence of the general formula MXXLXXXVXXLXXXQXXLXXXVXXLXXXV (SEQ ID NO: 105) wherein X is individually selected from any proteinogenic or non-proteinogenic amino acid residue. Such general formula may represent a typical sequence which is capable of forming a trimeric coiled coil.

The first polypeptide part may comprise an amino acid sequence of the general formula IXXIXXXIXXIXXXIXXIXXXIXXIXXXI (SEQ ID NO: 106) wherein X is individually selected from any proteinogenic or non-proteinogenic amino acid residue. Such general formula may represent a typical sequence which is capable of forming a tetrameric coiled coil.

The first polypeptide part may comprise an amino acid sequence of the general formula LXXIXXXLXXIXXXLXXIXXXLXXI (SEQ ID NO: 107) wherein X is individually selected from any proteinogenic or non-proteinogenic amino acid residue. Such general formula may represent a typical sequence which is capable of forming a tetrameric coiled coil.

The first polypeptide part may comprise an amino acid sequence of the general formula IXXXLXXIXXXLXXIXXXLXXIXXXL (SEQ ID NO: 108) wherein X is individually selected from any proteinogenic or non-proteinogenic amino acid residue. Such general formula may represent a typical sequence which is capable of forming a hexameric coiled coil.

The inventors have identified mutants of the GCN4p1 leucine zipper which surprisingly are capable of forming trimeric or tetrameric constructs, such as a trimeric or tetrameric coiled coil. The inventors have found that modification of the GCN4p1 sequence to include glutamine in place of an asparagine at position 16 (N16Q mutation) of the GCN4p1 sequence was found to provide a trimeric construct of peptides (Example 3, GCN4p1(NQ)). Thus, a polypeptide comprising GCN4p1(NQ) as the first polypeptide part was found to form an oligomeric state higher than a dimer.

Modification of the GCN4p1 sequence to include the following mutations (M2I, L5I, V9I, L12I, N16I, L19I, V23I, L26I, and V30I) was found to provide a tetrameric construct of the peptides (Example 3, GCN4p1(LI)) or a trimeric construct (Example 7, GCN4p1(LI). Thus, a polypeptide comprising GCN4p1(LI) as the first polypeptide part was found to form an oligomeric state higher than a dimer.

Modification of the GCN4p1 sequence to include two proline residues at position 7 and 14 was performed to disrupt the helical conformation of the GCN4p1 sequence and thereby disrupt the oligomerization, such as disrupt the coiled coil formation. The GCN4p1(7P14P) sequence was included in the study as a monomeric negative control to allow comparison of the polypeptides of the disclosure with monomeric polypeptides.

Modification of the GCN4p1 sequence to include the following mutations (L5I, V9L, L12I, N16L, L19I, V23L, L26I, and V30L) was found to provide a tetrameric construct of the peptides (Example 7, GCN4p1(ILI)). Thus, a polypeptide comprising GCN4p1(ILI) as the first polypeptide part was found to form an oligomeric state higher than a dimer.

In one embodiment, the first polypeptide part is selected from the group consisting of GCN4p1(NQ), GCN4p1(LI), GCN4p1(ILI), CC-Tet, CC-Hex2, ATF7-pII, ATF2-pII, NRP-pII, PIX-pII, HLF-pII, DBP-pII, TEF-pII, NRBI-pII, CREB4-pII, CREBH-pII, and MAT2-pII.

In one embodiment, the first polypeptide part is selected from the group consisting of ATF7-pII, ATF2-pII, NRP-pII, PIX-pII, HLF-pII, DBP-pII, TEF-pII, NRBI-pII, CREB4-pII, and CREBH-pII.

In one embodiment, the first polypeptide part is selected from the group consisting of GCN4p1(NQ), GCN4p1(LI), GCN4p1(ILI), CC-Tet, CC-Hex2, and ATF7-pII.

In one embodiment, the first polypeptide part is selected from the group consisting of GCN4p1(NQ), GCN4p1(LI), GCN4p1(ILI), CC-Tet, and CC-Hex2.

In one embodiment, the first polypeptide part is selected from the group consisting of GCN4p1(NQ), GCN4p1(LI), CC-Tet, and CC-Hex2.

In one embodiment, the first polypeptide part is GCN4p1(NQ) or GCN4p1(LI).

In one embodiment, the first polypeptide part is selected from the group consisting of GCN4p1(LI) and GCN4p1(ILI).

In one embodiment, the first polypeptide part has an amino acid sequence of RMKQLEDKVEELLSKQYHLENEVARLKKLV (SEQ ID NO: 67, GCN4p1(NQ)). In one embodiment, the first polypeptide part has an amino acid sequence of SEQ ID NO: 67 and is capable of forming an oligomeric state higher than 2, such as a trimer, such as a homotrimer, such as a coiled coil homotrimer.

In one embodiment, the first polypeptide part has an amino acid sequence of RIKQIEDKIEEILSKIYHIENEIARIKKLI (SEQ ID NO: 68, GCN4p1(LI)). In one embodiment, the first polypeptide part has an amino acid sequence of SEQ ID NO: 68 and is capable of forming an oligomeric state higher than 2, such as a tetramer, such as a homotetramer, such as a coiled coil homotetramer.

In one embodiment, the first polypeptide part has an amino acid sequence of RIKQIEDKIEEILSKIYHIENEIARIKKLI (SEQ ID NO: 68, GCN4p1(LI)). In one embodiment, the first polypeptide part has an amino acid sequence of SEQ ID NO: 68 and is capable of forming an oligomeric state higher than 2, such as a trimer, such as a homotrimer, such as a coiled coil homotrimer.

In one embodiment, the first polypeptide part has an amino acid sequence of RMKQIEDKLEEILSKLYHIENELARIKKLL (SEQ ID NO: 147, GCN4p1(ILI)). In one embodiment, the first polypeptide part has an amino acid sequence of SEQ ID NO: 147 and is capable of forming an oligomeric state higher than 2, such as a tetramer, such as a homotetramer, such as a coiled coil homotetramer.

In one embodiment, the first polypeptide part has an amino acid sequence of GELAAIKQELAAIKKELAAIKWELAAIKQ (SEQ ID NO: 69, CC-Tet, PDB: 3R4A). In one embodiment, the first polypeptide part has an amino acid sequence of SEQ ID NO: 69 and is capable of forming an oligomeric state higher than 2, such as a tetramer, such as a homotetramer, such as a coiled coil homotetramer.

In one embodiment, the first polypeptide part has an amino acid sequence of GELAAIKQELAAIKKELAAIKWELAAIKQ (SEQ ID NO: 69, CC-Tet, PDB: 3R4A). In one embodiment, the first polypeptide part has an amino acid sequence of SEQ ID NO: 69 and is capable of forming an oligomeric state higher than 2, such as a trimer, such as a homotrimer, such as a coiled coil homotrimer.

In one embodiment, the first polypeptide part has an amino acid sequence of GEIAKSLKEIAKSLKEIAWSLKEIAKSLK (SEQ ID NO: 70, CC-Hex2, PDB: 4PN9). In one embodiment, the first polypeptide part has an amino acid sequence of SEQ ID NO: 70 and is capable of forming an oligomeric state higher than 2, such as a hexamer, such as a homohexamer, such as a coiled coil homohexamer.

In one embodiment, the first polypeptide part has an amino acid sequence of VSSIEKKIEEITSQIIQISNEITLIRNEIAQIKQ (SEQ ID NO: 154, ATF7-pII). In one embodiment, the first polypeptide part has an amino acid sequence of SEQ ID NO: 154 and is capable of forming an oligomeric state higher than 2, such as a trimer, such as a homotrimer, such as a coiled coil homotrimer.

Second Polypeptide Part

In one embodiment, the second polypeptide part of the polypeptide encoded by the polynucleotide of the present disclosure is a peptide which is capable of binding to a PDZ domain. Such peptide ligand may be derived from the three to six C-terminal amino acid residues of an endogenous PDZ ligand protein. The peptide ligand may comprise a PDZ domain binding motif (PBM). Thus, in one embodiment, the second polypeptide part is consisting of or comprising an amino acid sequence selected from the group consisting of Σ-¥-ψ, ψ-¥-ψ, and ϕ-¥-ψ, wherein
Σ is Thr, Cys or Ser;
¥ is any proteinogenic amino acid;
ψ is any hydrophobic amino acid; and
ϕ is Asp or Glu.

PDZ domain binding motifs (PBM) may be divided into three groups, Class I PBM, Class II PBM and Class III PBM. The different classes of PBMs show different selectivity towards PDZ domains of different proteins.

In one embodiment, the second polypeptide part is a Class I PBM comprising or consisting of a sequence of Σ-¥-ψ, wherein
Σ is Thr, Cys or Ser,
¥ is any proteinogenic amino acid and
ψ is any hydrophobic amino acid,
   such as comprising or consisting of a sequence selected from the group consisting of TDV, TPV, and TSV. In one embodiment, the second polypeptide part comprises a Class I PBM and is consisting of or comprising a sequence selected from the group consisting of IETDV, RRTTPV, and YKQTSV.

In one embodiment, the second polypeptide part is a Class II PBM comprising or consisting of a sequence ψ-¥-ψ wherein
¥ is any proteinogenic amino acid and
ψ is any hydrophobic amino acid,
   such as comprising or consisting of the sequence LKV, IRV, IIV, VRV, LRV. In one embodiment, the second polypeptide part comprises a Class II PBM and is consisting of or comprising a sequence of HWLKV, FEIRV, NSIIV, NSVRV, NSLRV, NSIRV, NYIIV, NYIRV, TSIRV, YIIV, SVRV, EIRV, LRV, IIV, VRV, and IRV.

In one embodiment, the second polypeptide part is a Class III PBM comprising or consisting of a sequence of ϕ-¥-ψ, wherein
ϕ is Asp or Glu,
¥ is any proteinogenic amino acid and
ψ is any hydrophobic amino acid,
   such as comprising or consisting of a sequence selected from the group consisting of ESV, DSV, DYV, EMF, and DGA. In one embodiment, the second polypeptide part comprises a Class III PBM and is consisting of or comprising a sequence selected from the group consisting of WGESV, KVDSV, GKDYV, RKDYV, TAEMF and QEDGA.

In one embodiment, the second polypeptide part is selected from the group consisting of HWLKV, NSIRV, IETDV, RRTTPV, YKQTSV, and WGESV.

In one embodiment, the second polypeptide part is selected from the group consisting of HWLKV, IETDV, and RRTTPV.

In one embodiment, the second polypeptide part is HWLKV or NSIRV.

In one embodiment, the second polypeptide part is HWLKV or NSVRV.

In one embodiment, the second polypeptide part is HWLKV.

In one embodiment, the second polypeptide part is IETDV or RRTTPV.

In one embodiment, the second polypeptide part is IETDV.

In one embodiment, the second polypeptide part is WGESV.

In one embodiment, the second polypeptide part is selected from the group consisting of HWLKV, FEIRV, NSIIV, NSVRV, NSLRV, NSIRV, NYIIV, NYIRV, TSIRV, YIIV, SVRV, EIRV, LRV, IIV, VRV, and IRV.

In one embodiment, the second polypeptide part is selected from the group consisting of HWLKV, NSVRV, NSLRV, NSIRV, TSIRV, EIRV, YIIV, IIV, and IRV.

In one embodiment, the second polypeptide part is selected from the group consisting of NSVRV, NSLRV, NSIRV, TSIRV, EIRV, YIIV, IIV, and IRV.

In one embodiment, the second polypeptide part is selected from the group consisting of NSIIV, NSVRV, NSLRV, NSIRV, YIIV, SVRV, and LRV.

In one embodiment, the second polypeptide part is selected from the group consisting of FEIRV, NSIIV, NSVRV, NSLRV, NSIRV, YIIV, SVRV, VRV, and LRV.

In one embodiment, the second polypeptide part is HWLKV, NSVRV or NSIRV.

In one embodiment, the second polypeptide part is RRTTPV or YKQTSV.

In one embodiment, the second polypeptide part is HWLKV or IETDV.

In one embodiment, the second polypeptide part comprises or consists of an amino acid sequence of the general formula: X₁X₂X₃X₄X₅X₆;
wherein
X₁ is Y, R or is absent;
X₂ is R, K, I or is absent;
X₃ is T, E, Q; or is absent;
X₄ is T;
X₅ is D, S or P; and
X₆ is V.

In one embodiment, the second polypeptide part is comprising or consisting of an amino acid sequence of the general formula: X₁X₂X₃X₄X₅;
wherein
X₁ is H, N, F, or T, or is absent;
X₂ is W, S, E, or Y; or is absent;
X₃ is L, V, or I;
X₄ is K, I, or R; and
X₅ is V.

In one embodiment, the second polypeptide part is comprising or consisting of an amino acid sequence of the general formula: X₁X₂X₃X₄X₅;
wherein
X₁ is N, F, or T, or is absent;
X₂ is S, E, or Y; or is absent;
X₃ is V, L or I;
X₄ is I or R; and
X₅ is V.

In one embodiment, the second polypeptide part is comprising or consisting of an amino acid sequence of the general formula: X₁X₂X₃X₄X₅;
wherein
X₁ is N or T, or is absent;
X₂ is S, E, or Y; or is absent;
X₃ is V, L or I;
X₄ is I or R; and
X₅ is V.

In one embodiment, the second polypeptide part is comprising or consisting of an amino acid sequence of the general formula: X₁X₂X₃X₄X₅;
wherein
X₁ is N or F, or is absent;
X₂ is S, E, or Y; or is absent;
X₃ is V, L or I;
X₄ is I or R; and
X₅ is V.

Connectivity

The first polypeptide part and the second polypeptide part encoded by the polynucleotide of the present disclosure may be operably linked via a peptide linker of be directly fused to one another. In any event, the two polypeptide parts form part of one polypeptide chain. In one embodiment, the first polypeptide part is positioned N-terminal to the second polypeptide part.

The first polypeptide part and the second polypeptide part encoded by the polynucleotide of the present disclosure may optionally be operably linked via a linker.

In one embodiment, the first polypeptide part and the second polypeptide part are operably linked via a linker. In one embodiment, the linker is a peptide linker, such as a glycine serine (GS) linker.

In one embodiment, the linker is a glycine serine linker selected from the group consisting of GGS (gLinker2, GS2), GGGS (gLinker3, GS3, SEQ ID NO: 71), GGGGS (gLinker4, GS4, SEQ ID NO: 72), GGGGSG (gLinker5, GS5, SEQ ID NO: 73), GGGGSGG (gLinker6, GS6, SEQ ID NO: 74). In a preferred embodiment, the linker is GGGGS (glinker4, GS4, SEQ ID NO: 72).

In one embodiment, the linker comprises 1 to 12 repeats of the GS linker, such as 1 to 12 repeats of GS4.

Tag

The polypeptide encoded by the polynucleotide of the present disclosure may further comprise a tag. In one embodiment, the tag is conjugated to the N-terminal end of the first polypeptide part. In one embodiment, the tag consists of or comprises an amino acid sequence, which may be operably linked to the polypeptide of the present disclosure. The tag may be used for visualization and/or purification of the polypeptide.

Thus, in one embodiment, the polypeptide of the present disclosure further comprises a tag. In one embodiment, the tag is conjugated to the N-terminus of the polypeptide, optionally via a linker. In one embodiment med linker is a GS linker as defined herein or a 6-aminohexanoic acid (Ahx) linker.

In one embodiment, the tag is selected from the group consisting of HA-tag, Myc-tag and His-tag. In one embodiment, the tag is a HA-tag. In one embodiment, the tag is a Myc-tag or a His-tag.

In one embodiment, the tag is conjugated to the polypeptide following expression and purification of the polypeptide. In one embodiment, the tag is conjugated to the polypeptide following synthesis of the polypeptide, such as synthesis by solid phase peptide synthesis.

In one embodiment, the tag is a Biotin tag. In one embodiment, the biotin tag is conjugated to the N-terminal end of the polypeptide via a 6-aminohexanoic acid (Ahx) linker.

In one embodiment, the tag is used for detection. The tag may be selected from fluorescent protein or an antibody tag. In one embodiment, the detectable tag is selected from the group consisting of GFP, enhanced GFP (EGFP) and TdTomato. In one embodiment, the antibody tag is selected from HA-tag, myc-tag, His-tag or biotin.

In one embodiment, the tag is conjugated to the N-terminus of the first polypeptide. In one embodiment, an HA-tag and a GS linker is added to the N terminus of the first polypeptide, for identification and tracking purposes. In one embodiment, the first polypeptide is further conjugated to biotin. In another embodiment, the biotin is attached to the N-terminus of the first polypeptide.

CPP

The polypeptide encoded by the polynucleotide of the present disclosure may further comprises a cell penetrating peptide (CPP). The CPP may be operably linked to the polypeptide.

In one embodiment, the CPP is operably linked to the polypeptide via a linker, such as a polypeptide linker, such as a glycine serine linker.

In one embodiment, the CPP is positioned N-terminal to the first and the second polypeptide parts.

In one embodiment, the CPP is selected from the group consisting of TAT, polyarginine, TP10, MAP and PNT.

Preferred Polypeptides

The polypeptide encoded by the polynucleotide of the present disclosure may comprise a sequence selected from the group consisting of SEQ ID NO: 75, 76, 77, 78, 79, 80, 81, 82, 83, 84, 85, 86, 87, 88, 89, 90, 91, 92, 93, 94, 95, 96, 97, 98, 144, 146, 148, 149, 150, 151, 152, 194 and 195.

In embodiments, the first polypeptide part is selected from the group consisting of: SEQ ID NO: 67, 68, 69, 70, 147, 154, and any one of 159-168, the linker is selected from GGS, and any one of SEQ ID NO: 71-74, and the second polypeptide is selected from any one of SEQ ID NO: 5-64 or IIV, IRV, VIV, VRV, and LRV.

In preferred embodiments the first polypeptide part is selected from the group consisting of: SEQ ID NO: 67, 68, 69, 70, 147, and 154, the linker is SEQ ID NO: 72, and the second polypeptide is selected from any one of SEQ ID NO: 5-64 or IIV, IRV, VIV, VRV, and LRV.

The polypeptide encoded by the polynucleotide of the present disclosure may comprise a sequence selected from the list provided in the below table.

| | Name |
|---|---|
| SEQ ID NO: | Peptide Sequence |
| SEQ ID NO: 75 | GCN4p1(NQ)-GS4-HWLKV |
| | RMKQLEDKVEELLSKQYHLENEVARLKKLVGGGG |
| | SHWLKV |
| SEQ ID NO: 76 | GCN4p1(NQ)-GS4-NSIRV |
| | RMKQLEDKVEELLSKQYHLENEVARLKKLVGGGG |
| | SNSIRV |
| SEQ ID NO: 77 | GCN4p1(NQ)-GS4-IETDV |
| | RMKQLEDKVEELLSKQYHLENEVARLKKLVGGGG |
| | SIETDV |
| SEQ ID NO: 78 | GCN4p1(NQ)-GS4-RRTTPV |
| | RMKQLEDKVEELLSKQYHLENEVARLKKLVGGGG |
| | SRRTTPV |
| SEQ ID NO: 79 | GCN4p1(NQ)-GS4-YKQTSV |
| | RMKQLEDKVEELLSKQYHLENEVARLKKLVGGGG |
| | SYKQTSV |
| SEQ ID NO: 80 | GCN4p1(NQ)-GS4-WGESV |
| | RMKQLEDKVEELLSKQYHLENEVARLKKLVGGGG |
| | SWGESV |
| SEQ ID NO: 81 | GCN4p1(LI)-GS4-HWLKV |
| | RIKQIEDKIEEILSKIYHIENEIARIKKLIGGGG |
| | SHWLKV |
| SEQ ID NO: 82 | GCN4p1(LI)-GS4-NSIRV |
| | RIKQIEDKIEEILSKIYHIENEIARIKKLIGGGG |
| | SNSIRV |
| SEQ ID NO: 83 | GCN4p1(LI)-GS4-IETDV |
| | RIKQIEDKIEEILSKIYHIENEIARIKKLIGGGG |
| | SIETDV |
| SEQ ID NO: 84 | GCN4p1(LI)-GS4-RRTTPV |
| | RIKQIEDKIEEILSKIYHIENEIARIKKLIGGGG |
| | SRRTTPV |
| SEQ ID NO: 85 | GCN4p1(LI)-GS4-YKQTSV |
| | RIKQIEDKIEEILSKIYHIENEIARIKKLIGGGG |
| | SYKQTSV |
| SEQ ID NO: 86 | GCN4p1(LI)-GS4-WGESV |
| | RIKQIEDKIEEILSKIYHIENEIARIKKLIGGGG |
| | SWGESV |
| SEQ ID NO: 87 | CC-Tet-GS4-HWLKV |
| | GELAAIKQELAAIKKELAAIKWELAAIKQGGGGS |
| | HWLKV |
| SEQ ID NO: 88 | CC-Tet-GS4-NSIRV |
| | GELAAIKQELAAIKKELAAIKWELAAIKQGGGGS |
| | NSIRV |
| SEQ ID NO: 89 | CC-Tet-GS4-IETDV |
| | GELAAIKQELAAIKKELAAIKWELAAIKQGGGGS |
| | IETDV |
| SEQ ID NO: 90 | CC-Tet-GS4-RRTTPV |
| | GELAAIKQELAAIKKELAAIKWELAAIKQGGGGS |
| | RRTTPV |
| SEQ ID NO: 91 | CC-Tet-GS4-YKQTSV |
| | GELAAIKQELAAIKKELAAIKWELAAIKQGGGGS |
| | YKQTSV |
| SEQ ID NO: 92 | CC-Tet-GS4-WGESV |
| | GELAAIKQELAAIKKELAAIKWELAAIKQGGGGS |
| | WGESV |
| SEQ ID NO: 93 | CC-Hex2-GS4-HWLKV |
| | GEIAKSLKEIAKSLKEIAWSLKEIAKSLKGGGGS |
| | HWLKV |
| SEQ ID NO: 94 | CC-Hex2-GS4-NSIRV |
| | GEIAKSLKEIAKSLKEIAWSLKEIAKSLKGGGGS |
| | NSIRV |
| SEQ ID NO: 95 | CC-Hex2-GS4-IETDV |
| | GEIAKSLKEIAKSLKEIAWSLKEIAKSLKGGGGS |
| | IETDV |
| SEQ ID NO: 96 | CC-Hex2-GS4-RRTTPV |
| | GEIAKSLKEIAKSLKEIAWSLKEIAKSLKGGGGS |
| | RRTTPV |
| SEQ ID NO: 97 | CC-Hex2-GS4-YKQTSV |
| | GEIAKSLKEIAKSLKEIAWSLKEIAKSLKGGGGS |
| | YKQTSV |
| SEQ ID NO: 98 | CC-Hex2-GS4-WGESV |
| | GEIAKSLKEIAKSLKEIAWSLKEIAKSLKGGGGS |
| | WGESV |
| SEQ ID NO: 194 | ATF7-GS4-HWLKV |
| | VSSIEKKIEEITSQIIQISNEITLIRNEIAQIKQ |
| | GGGGSHWLKV |
| SEQ ID NO: 195 | ATF7-GS4-IETDV |
| | VSSIEKKIEEITSQIIQISNEITLIRNEIAQIKQ |
| | GGGGSIETDV |

In one embodiment, the polypeptide encoded by the polynucleotide of the present disclosure comprises a sequence selected from the list provided in the below table.

| | Name |
|---|---|
| SEQ ID NO: | Peptide Sequence |
| SEQ ID NO: 75 | GCN4p1(NQ)-GS4-HWLKV |
| | RMKQLEDKVEELLSKQYHLENEVARLKKLVGGGGS |
| | HWLKV |
| SEQ ID NO: 77 | GCN4p1(NQ)-GS4-IETDV |
| | RMKQLEDKVEELLSKQYHLENEVARLKKLVGGGGS |
| | IETDV |
| SEQ ID NO: 78 | GCN4p1(NQ)-GS4-RRTTPV |
| | RMKQLEDKVEELLSKQYHLENEVARLKKLVGGGGS |
| | RRTTPV |
| SEQ ID NO: 81 | GCN4p1(LI)-GS4-HWLKV |
| | RIKQIEDKIEEILSKIYHIENEIARIKKLIGGGGS |
| | HWLKV |
| SEQ ID NO: 83 | GCN4p1(LI)-GS4-IETDV |
| | RIKQIEDKIEEILSKIYHIENEIARIKKLIGGGGS |
| | IETDV |
| SEQ ID NO: 84 | GCN4p1(LI)-GS4-RRTTPV |
| | RIKQIEDKIEEILSKIYHIENEIARIKKLIGGGGS |
| | RRTTPV |
| SEQ ID NO: 87 | CC-Tet-GS4-HWLKV |
| | GELAAIKQELAAIKKELAAIKWELAAIKQGGGGSH |
| | WLKV |
| SEQ ID NO: 89 | CC-Tet-GS4-IETDV |
| | GELAAIKQELAAIKKELAAIKWELAAIKQGGGGSI |
| | ETDV |
| SEQ ID NO: 90 | CC-Tet-GS4-RRTTPV |
| | GELAAIKQELAAIKKELAAIKWELAAIKQGGGGSR |
| | RTTPV |
| SEQ ID NO: 93 | CC-Hex2-GS4-HWLKV |
| | GEIAKSLKEIAKSLKEIAWSLKEIAKSLKGGGGSH |
| | WLKV |
| SEQ ID NO: 95 | CC-Hex2-GS4-IETDV |
| | GEIAKSLKEIAKSLKEIAWSLKEIAKSLKGGGGSI |
| | ETDV |
| SEQ ID NO: 96 | CC-Hex2-GS4-RRTTPV |
| | GEIAKSLKEIAKSLKEIAWSLKEIAKSLKGGGGSR |
| | RTTPV |

In one embodiment, the polypeptide encoded by the polynucleotide of the present disclosure comprises a sequence selected the group consisting of SEQ ID NO:75-99, 144, 146-152, 194 and 195.

In one embodiment, the polypeptide encoded by the polynucleotide of the present disclosure comprises a sequence selected the group consisting of SEQ ID NO: 75, SEQ ID NO: 77, SEQ ID NO: 78, SEQ ID NO: 80, SEQ ID NO: 81, SEQ ID NO: 83, SEQ ID NO: 84, SEQ ID NO: 86, SEQ ID NO: 87, SEQ ID NO: 89, SEQ ID NO: 92, SEQ ID NO: 93, SEQ ID NO: 95, SEQ ID NO: 96, SEQ ID NO: 98, SEQ ID NO: 152, SEQ ID NO: 157, SEQ ID NO:194, and SEQ ID NO: 195.

In another embodiment, the polypeptide encoded by the polynucleotide of the present disclosure comprises a sequence selected the group consisting of SEQ ID NO: 81, SEQ ID NO: 83, SEQ ID NO: 84, SEQ ID NO: 86, SEQ ID NO: 87, SEQ ID NO: 89, SEQ ID NO: 90, SEQ ID NO:92, SEQ ID NO: 157, SEQ ID NO: 194, and SEQ ID NO: 195.

In a preferred embodiment, the polypeptide encoded by the polynucleotide of the present disclosure comprises a sequence selected the group consisting of SEQ ID NO: 81, SEQ ID NO: 83, SEQ ID NO: 84, SEQ ID NO: 87, SEQ ID NO: 89 and SEQ ID NO: 90.

In another preferred embodiment, the polypeptide encoded by the polynucleotide of the present disclosure comprises a sequence selected the group consisting of SEQ ID NO: 75, SEQ ID NO: 81, SEQ ID NO: 87, SEQ ID NO: 93, and SEQ ID NO: 194.

In yet another preferred embodiment, the polypeptide encoded by the polynucleotide of the present disclosure comprises a sequence selected the group consisting of SEQ ID NO: 77, SEQ ID NO: 78, SEQ ID NO: 83, SEQ ID NO: 84, SEQ ID NO: 89, SEQ ID NO: 95, SEQ ID NO: 96, and SEQ ID NO: 195.

In a further embodiment, the polypeptide encoded by the polynucleotide of the present disclosure comprises a sequence selected the group consisting of SEQ ID NO:80, SEQ ID NO: 86, SEQ ID NO: 92, SEQ ID NO:98, SEQ ID NO:157.

In a preferred embodiment, the polypeptide encoded by the polynucleotide of the present disclosure comprises a sequence selected the group consisting of SEQ ID NO: 81, SEQ ID NO: 83, SEQ ID NO: 84, SEQ ID NO:86, and SEQ ID NO: 157.

In an especially preferred exemplified embodiment, the polypeptide encoded by the polynucleotide of the present disclosure comprises a sequence selected the group consisting of SEQ ID NO: 81, SEQ ID NO: 83, SEQ ID NO: 84.

In a further preferred exemplified embodiment, the polypeptide encoded by the polynucleotide of the present disclosure comprises a sequence of SEQ ID NO: 81.

In a yet further preferred exemplified embodiment, the polypeptide encoded by the polynucleotide of the present disclosure comprises a sequence of SEQ ID NO: 83.

In yet another preferred exemplified embodiment, the polypeptide encoded by the polynucleotide of the present disclosure comprises a sequence of SEQ ID NO: 84.

Polynucleotide

In one embodiment, the polynucleotide of the present disclosure comprises a sequence selected from the group consisting of SEQ ID NO: 109, 110, 111, 112, 173, 180, 182, 183, 184, 185, 186, 187, 188, 189, 190, and 191. The polynucleotide sequences are disclosed without start codon and/or stop codon, however, these will needless to say be included in the sequence for expression of the polypeptide encoded by the sequence.

In one embodiment, the polynucleotide of the present disclosure comprises a sequence selected from the list provided in the below table.

| | Name |
|---|---|
| SEQ ID NO: | DNA Sequence |
| SEQ ID NO: 109 | GCN4p1(NQ) |
| | cgcatgaaacagctggaagataaagtggaagaac |
| | tgctgagcaaacagtatcatctggaaaacgaagt |
| | ggcgcgcctgaaaaaactggtg |
| SEQ ID NO: 110 | GCN4p1(LI) |
| | cgcatcaaacagatcgaagataaaatcgaagaaa |
| | tcctgagcaaaatctatcatatcgaaaacgaaat |
| | cgcgcgcatcaaaaaactgatc |
| SEQ ID NO: 111 | CC-Tet |
| | ggcgagctggccgccatcaagcaggagctggccg |
| | ccatcaagaaggagctggccgccatcaagtggga |
| | gctggccgccatcaagcag |
| SEQ ID NO: 112 | CC-Hex2 |
| | ggcgagatcgccaagagcctgaaggagatcgcca |
| | agagcctgaaggagatcgcctggagcctgaagga |
| | gatcgccaagagcctgaag |

In one embodiment, the polynucleotide of the present disclosure comprises a sequence selected from the group consisting of SEQ ID NO: 113, 114, 115, 116, 117, 118, 119, and 181.

In one embodiment, the polynucleotide of the present disclosure comprises a sequence selected from the list provided in the below table.

| | Name |
|---|---|
| SEQ ID NO: | DNA Sequence |
| SEQ ID NO: 113 | HWLKV |
| | cattggctgaaagtg |
| SEQ ID NO: 114 | NSIRV |
| | aacagcatcagggtg |
| SEQ ID NO: 115 | IETDV |
| | atcgagaccgacgtg |
| SEQ ID NO: 116 | RRTTPV |
| | aggaggaccacccccgtg |
| SEQ ID NO: 117 | YKQTSV |
| | tacaagcagaccagcgtg |
| SEQ ID NO: 118 | WGESV |
| | tggggcgagagcgtg |
| SEQ ID NO: 181 | NSVRV |
| | aacagcgtgagggtg |

In one embodiment, the polynucleotide of the present disclosure comprises a sequence selected from the group consisting of SEQ ID NO: 120, 121, 122, 123, 124, 125, 126, 127, 128, 129, 130, 131, 132, 133, 134, 135, 136, 137, 138, 139, 140, 141, 142, 143, 169, 171, 172, 174, 175, 176, 177, 178, 192, and 193.

In one embodiment, the polynucleotide of the present disclosure comprises a sequence selected from the list provided in the below table.

| | Name |
|---|---|
| SEQ ID NO: | DNA Sequence |
| SEQ ID NO: 120 | GCN4p1(NQ)-GS4-HWLKV |
| | cgcatgaaacagctggaagataaagtggaagaactgctgagcaaacagtatcatctg |
| | gaaaacgaagtggcgcgcctgaaaaaactggtgggcggcggcggcagccattggct |
| | gaaagtg |
| SEQ ID NO: 121 | GCN4p1(NQ)-GS4-NSIRV |
| | cgcatgaaacagctggaagataaagtggaagaactgctgagcaaacagtatcatctg |
| | gaaaacgaagtggcgcgcctgaaaaaactggtgggcggcggcggcagcaacagc |
| | atcagggtg |
| SEQ ID NO: 122 | GCN4p1(NQ)-GS4-IETDV |
| | cgcatgaaacagctggaagataaagtggaagaactgctgagcaaacagtatcatctg |
| | gaaaacgaagtggcgcgcctgaaaaaactggtgggcggcggcggcagcatcgaga |
| | ccgacgtg |
| SEQ ID NO: 123 | GCN4p1(NQ)-GS4-RRTTPV |
| | cgcatgaaacagctggaagataaagtggaagaactgctgagcaaacagtatcatctg |
| | gaaaacgaagtggcgcgcctgaaaaaactggtgggcggcggcggcagcaggagg |
| | accacccccgtg |
| SEQ ID NO: 124 | GCN4p1(NQ)-GS4-YKQTSV |
| | cgcatgaaacagctggaagataaagtggaagaactgctgagcaaacagtatcatctg |
| | gaaaacgaagtggcgcgcctgaaaaaactggtgggcggcggcggcagctacaagc |
| | agaccagcgtg |
| SEQ ID NO: 125 | GCN4p1(NQ)-GS4-WGESV |
| | cgcatgaaacagctggaagataaagtggaagaactgctgagcaaacagtatcatctg |
| | gaaaacgaagtggcgcgcctgaaaaaactggtgggcggcggcggcagctggggcg |
| | agagcgtg |
| SEQ ID NO: 126 | GCN4p1(LI)-GS4-HWLKV |
| | cgcatcaaacagatcgaagataaaatcgaagaaatcctgagcaaaatctatcatatc |
| | gaaaacgaaatcgcgcgcatcaaaaaactgatcggcggcggcggcagccattggctg |
| | aaagtg |
| SEQ ID NO: 127 | GCN4p1(LI)-GS4-NSIRV |
| | cgcatcaaacagatcgaagataaaatcgaagaaatcctgagcaaaatctatcatatc |
| | gaaaacgaaatcgcgcgcatcaaaaaactgatcggcggcggcggcagcaacagcatc |
| | agggtg |
| SEQ ID NO: 128 | GCN4p1(LI)-GS4-IETDV |
| | cgcatcaaacagatcgaagataaaatcgaagaaatcctgagcaaaatctatcatatc |
| | gaaaacgaaatcgcgcgcatcaaaaaactgatcggcggcggcggcagcatcgagacc |
| | gacgtg |
| SEQ ID NO: 129 | GCN4p1(LI)-GS4-RRTTPV |
| | cgcatcaaacagatcgaagataaaatcgaagaaatcctgagcaaaatctatcatatc |
| | gaaaacgaaatcgcgcgcatcaaaaaactgatcggcggcggcggcagcaggaggacc |
| | acccccgtg |
| SEQ ID NO: 130 | GCN4p1(LI)-GS4-YKQTSV |
| | cgcatcaaacagatcgaagataaaatcgaagaaatcctgagcaaaatctatcatatc |
| | gaaaacgaaatcgcgcgcatcaaaaaactgatcggcggcggcggcagctacaagcag |
| | accagcgtg |
| SEQ ID NO: 131 | GCN4p1(LI)-GS4-WGESV |
| | cgcatcaaacagatcgaagataaaatcgaagaaatcctgagcaaaatctatcatatc |
| | gaaaacgaaatcgcgcgcatcaaaaaactgatcggcggcggcggcagctggggcgag |
| | agcgtg |
| SEQ ID NO: 132 | CC-Tet-GS4-HWLKV |
| | ggcgagctggccgccatcaagcaggagctggccgccatcaagaaggagctggccgcc |
| | atcaagtgggagctggccgccatcaagcagggcggcggcggcagccactggctgaag |
| | gtg |
| SEQ ID NO: 133 | CC-Tet-GS4-NSIRV |
| | ggcgagctggccgccatcaagcaggagctggccgccatcaagaaggagctggccgcc |
| | atcaagtgggagctggccgccatcaagcagggcggcggcggcagcaacagcatcagg |
| | gtg |
| SEQ ID NO: 134 | CC-Tet-GS4-IETDV |
| | ggcgagctggccgccatcaagcaggagctggccgccatcaagaaggagctggccgcc |
| | atcaagtgggagctggccgccatcaagcagggcggcggcggcagcatcgagaccgac |
| | gtg |
| SEQ ID NO: 135 | CC-Tet-GS4-RRTTPV |
| | ggcgagctggccgccatcaagcaggagctggccgccatcaagaaggagctggccgcc |
| | atcaagtgggagctggccgccatcaagcagggcggcggcggcagcaggaggaccacc |
| | cccgtg |
| SEQ ID NO: 136 | CC-Tet-GS4-YKQTSV |
| | ggcgagctggccgccatcaagcaggagctggccgccatcaagaaggagctggccgcc |
| | atcaagtgggagctggccgccatcaagcagggcggcggcggcagctacaagcagacc |
| | agcgtg |
| SEQ ID NO: 137 | CC-Tet-GS4-WGESV |
| | ggcgagctggccgccatcaagcaggagctggccgccatcaagaaggagctggccgcc |
| | atcaagtgggagctggccgccatcaagcagggcggcggcggcagctggggcgagagc |
| | gtg |
| SEQ ID NO: 138 | CC-Hex2-GS4-HWLKV |
| | ggcgagatcgccaagagcctgaaggagatcgccaagagcctgaaggagatcgcctgg |
| | agcctgaaggagatcgccaagagcctgaagggcggcggcggcagccactggctgaag |
| | gtg |
| SEQ ID NO: 139 | CC-Hex2-GS4-NSIRV |
| | ggcgagatcgccaagagcctgaaggagatcgccaagagcctgaaggagatcgcctgg |
| | agcctgaaggagatcgccaagagcctgaagggcggcggcggcagcaacagcatcagg |
| | gtg |
| SEQ ID NO: 140 | CC-Hex2-GS4-IETDV |
| | ggcgagatcgccaagagcctgaaggagatcgccaagagcctgaaggagatcgcctgg |
| | agcctgaaggagatcgccaagagcctgaagggcggcggcggcagcatcgagaccgac |
| | gtg |
| SEQ ID NO: 141 | CC-Hex2-GS4-RRTTPV |
| | ggcgagatcgccaagagcctgaaggagatcgccaagagcctgaaggagatcgcctgg |
| | agcctgaaggagatcgccaagagcctgaagggcggcggcggcagcaggaggaccacc |
| | cccgtg |
| SEQ ID NO: 142 | CC-Hex2-GS4-YKQTSV |
| | ggcgagatcgccaagagcctgaaggagatcgccaagagcctgaaggagatcgcctgg |
| | agcctgaaggagatcgccaagagcctgaagggcggcggcggcagctacaagcagacc |
| | agcgtg |
| SEQ ID NO: 143 | CC-Hex2-GS4-WGESV |
| | ggcgagatcgccaagagcctgaaggagatcgccaagagcctgaaggagatcgcctgg |
| | agcctgaaggagatcgccaagagcctgaagggcggcggcggcagctggggcgagagc |
| | gtg |

In one embodiment, the polynucleotide of the present disclosure comprises a sequence selected from the list provided in the below table.

| | Name |
|---|---|
| SEQ ID NO: | DNA Sequence |
| SEQ ID NO: 120 | GCN4p1(NQ)-GS4-HWLKV |
| | cgcatgaaacagctggaagataaagtggaagaac |
| | tgctgagcaaacagtatcatctggaaaacgaagt |
| | ggcgcgcctgaaaaaactggtgggcggcggcggc |
| | agccattggctgaaagtg |
| SEQ ID NO: 122 | GCN4p1(NQ)-GS4-IETDV |
| | cgcatgaaacagctggaagataaagtggaagaac |
| | tgctgagcaaacagtatcatctggaaaacgaagt |
| | ggcgcgcctgaaaaaactggtgggcggcggcggc |
| | agcatcgagaccgacgtg |
| SEQ ID NO: 123 | GCN4p1(NQ)-GS4-RRTTPV |
| | cgcatgaaacagctggaagataaagtggaagaac |
| | tgctgagcaaacagtatcatctggaaaacgaagt |
| | ggcgcgcctgaaaaaactggtgggcggcggcggc |
| | agcaggaggaccacccccgtg |
| SEQ ID NO: 126 | GCN4p1(LI)-GS4-HWLKV |
| | cgcatcaaacagatcgaagataaaatcgaagaaa |
| | tcctgagcaaaatctatcatatcgaaaacgaaat |
| | cgcgcgcatcaaaaaactgatcggcggcggcggc |
| | agccattggctgaaagtg |
| SEQ ID NO: 128 | GCN4p1(LI)-GS4-IETDV |
| | cgcatcaaacagatcgaagataaaatcgaagaaa |
| | tcctgagcaaaatctatcatatcgaaaacgaaat |
| | cgcgcgcatcaaaaaactgatcggcggcggcggc |
| | agcatcgagaccgacgtg |
| SEQ ID NO: 129 | GCN4p1(LI)-GS4-RRTTPV |
| | cgcatcaaacagatcgaagataaaatcgaagaaa |
| | tcctgagcaaaatctatcatatcgaaaacgaaat |
| | cgcgcgcatcaaaaaactgatcggcggcggcggc |
| | agcaggaggaccacccccgtg |
| SEQ ID NO: 132 | CC-Tet-GS4-HWLKV |
| | ggcgagctggccgccatcaagcaggagctggccg |
| | ccatcaagaaggagctggccgccatcaagtggga |
| | gctggccgccatcaagcagggcggcggcggcagc |
| | cactggctgaaggtg |
| SEQ ID NO: 134 | CC-Tet-GS4-IETDV |
| | ggcgagctggccgccatcaagcaggagctggccg |
| | ccatcaagaaggagctggccgccatcaagtggga |
| | gctggccgccatcaagcagggcggcggcggcagc |
| | atcgagaccgacgtg |
| SEQ ID NO: 135 | CC-Tet-GS4-RRTTPV |
| | ggcgagctggccgccatcaagcaggagctggccg |
| | ccatcaagaaggagctggccgccatcaagtggga |
| | gctggccgccatcaagcagggcggcggcggcagc |
| | aggaggaccacccccgtg |
| SEQ ID NO: 138 | CC-Hex2-GS4-HWLKV |
| | ggcgagatcgccaagagcctgaaggagatcgcca |
| | agagcctgaaggagatcgcctggagcctgaagga |
| | gatcgccaagagcctgaagggcggcggcggcagc |
| | cactggctgaaggtg |
| SEQ ID NO: 140 | CC-Hex2-GS4-IETDV |
| | ggcgagatcgccaagagcctgaaggagatcgcca |
| | agagcctgaaggagatcgcctggagcctgaagga |
| | gatcgccaagagcctgaagggcggcggcggcagc |
| | atcgagaccgacgtg |
| SEQ ID NO: 141 | CC-Hex2-GS4-RRTTPV |
| | ggcgagatcgccaagagcctgaaggagatcgcca |
| | agagcctgaaggagatcgcctggagcctgaagga |
| | gatcgccaagagcctgaagggcggcggcggcagc |
| | aggaggaccacccccgtg |

In one embodiment, the polynucleotide may comprise a sequence variant of a polynucleotide of the present disclosure, such as SEQ ID NO: 120, 121, 122, 123, 124, 125, 126, 127, 128, 129, 130, 131, 132, 133, 134, 135, 136, 137, 138, 139, 140, 141, 142, 143, 169, 171, 172, 174, 175, 176, 177, 178, 192, and 193, wherein the sequence variant has at least 70% sequence identity to said nucleotide sequence, such as at least 75% sequence identity, for example at least 80% sequence identity, such as at least 85% sequence identity, for example at least 90% sequence identity, such as at least 95% sequence identity, for example at least 96% sequence identity, such as at least 97% sequence identity, for example at least 98% sequence identity, such as at least 99% sequence identity to said nucleotide sequence.

In one embodiment, the polynucleotide may comprise a sequence variant of a polynucleotide of the present disclosure, wherein the sequence variant is codon optimized for expression in human beings.

The polynucleotide of the present disclosure may further comprise a promoter sequence.

In one embodiment, the polynucleotide further comprises a promoter that permits high expression in neurons, such as for example dorsal spinal horn neurons. In a preferred embodiment, said promoter is neuron-specific. In a most preferred embodiment, said promoter is a human synapsin promoter. In another embodiment, the promoter is a human Synapsin1 promoter.

In one embodiment, the promoter is a promoter specific for mammalian cells. In a further embodiment, the promoter is a promoter specific for neural cells. In yet a further embodiment, the promoter is a promoter specific for neurons.

In one embodiment, the promoter is a constitutive promoter, such as a constitutively active promoter selected from the group consisting of CAG, CBA, CMV, human UbiC, RSV, EF-1alpha, NSE, SV40, and Mt1.

In one embodiment, the promoter is an inducible promoter, such as an inducible promoter selected from the group consisting of Tet-On, Tet-Off, Mo-MLV-LTR, Mx1, progesterone, RU486, and Rapamycin-inducible promoter.

In one embodiment, the promoter is an activity-dependent promoter, such as an activity-dependent promoter selected from the group consisting of cFos, Arc, Npas4, and Egr1 promoters.

In one embodiment, the promoter is Robust Activity Marking (RAM) promoter. This promoter is described by Sorensen et al., 2016.

In another embodiment, the polynucleotide sequence of the present invention is regulated by a post-transcriptional regulatory element. In a preferred embodiment, said regulatory element is a Woodchuck hepatitis virus post-transcriptional regulatory element (WPRE).

Recombinant Expression Vector The polynucleotide of the present disclosure may be present in a vector, such as in an expression vector. Thus, in one embodiment, an expression vector is provided comprising the polynucleotide as disclosed herein. In one embodiment, the vector comprises a polynucleotide sequence encoding the polypeptide as disclosed herein.

Broadly, gene therapy seeks to transfer new genetic material to the cells of a patient with resulting therapeutic benefit to the patient. Such benefits include treatment or prophylaxis of a broad range of diseases and/or disorders.

In one embodiment, the vector is selected from the group consisting of RNA based vectors, DNA based vectors, lipid based vectors, polymer based vectors and colloidal gold particles.

In one embodiment, the vector is a viral vector, such as a virally derived DNA vector or a virally derived RNA vector.

Different viral vectors may be used for delivering genetic material into a host organism. In one embodiment, the vector is selected from papovavirus, adenovirus, vaccinia virus, adeno-associated virus (AAV), herpes virus, and retroviruses, such as lentivirus, HIV, SIV, FIV, EIAV, or MoMLV.

In one embodiment, the vector is selected from the group consisting of adenoviruses, recombinant adeno-associated viruses (rAAV), retroviruses, lentiviruses, adeno-associated viruses, herpesviruses, vaccinia viruses, foamy viruses, cytomegaloviruses, Semliki forest virus, poxviruses, RNA virus vector, and DNA virus vector.

In one embodiment, a preferred virus for treatment of disorders of the central nervous system is lentiviruses or adeno-associated viruses (AAV).

In a preferred embodiment, the vector is an adeno-associated virus (AAV).

Different serotypes of AAV exist. In one embodiment, the adeno associated vector (AAV) is selected from the group consisting of an AAV1 vector, an AAV2 vector, an AAV5, an AAV8, and an AAV9 vector.

In one embodiment the vector is an AAV1 vector. In one embodiment the vector is an AAV2 vector. In one embodiment the vector is an AAV5 vector. In one embodiment the vector is an AAV8 vector. In one embodiment the vector is an AAV9 vector.

In addition to using a specific serotype of the AAV, it may be possible to combine different serotypes, such as using the plasmid of one serotype packaged in the capsid of another serotype.

Thus, in one embodiment, the AAV is an AAV1 plasmid which is packaged in an AAV capsid other than an AAV1 capsid, such as packaged in an AAV2, AAV5, AAV8, or AAV9 capsid.

In one embodiment, the AAV is an AAV2 plasmid which is packaged in an AAV capsid other than an AAV2 capsid, such as packaged in an AAV1, AAV5, AAV8, or AAV9 capsid.

In one embodiment the AAV is an AAV5 plasmid which is packaged in an AAV capsid other than an AAV5 capsid, such as packaged in an AAV1, AAV2, AAV8, or AAV9 capsid.

In one embodiment the AAV is an AAV8 plasmid which is packaged in an AAV capsid other than an AAV8 capsid, such as packaged in an AAV1, AAV2, AAV5, or AAV9 capsid.

In one embodiment the AAV is an AAV9 plasmid which is packaged in an AAV capsid other than an AAV9 capsid, such as packaged in an AAV1, AAV2, AAV5, or AAV8 capsid.

In one embodiment, the vector based on AAV vectors can be of any serotype modified to express altered or novel coat proteins.

In one embodiment, the vector is based on any AAV serotype identified in humans, non-human primates, other mammalian species, or chimeric versions thereof.

AAV vectors may be prepared using two major principles, transfection of human cell line monolayer culture or free floating insect cells, however, any method for preparation and delivery of AAV to the central nervous system (CNS) known in the art may be used.

In one embodiment, the recombinant vector encodes a polypeptide as disclosed herein.

In one embodiment, following delivery of the polynucleotide of the present disclosure, such as delivery of a viral vector comprising the polynucleotide of the present disclosure into a living cell, the polynucleotide sequence is first transcribed, then translated into a single polypeptide (monomer). In one embodiment, the polypeptide is capable of self-assembling into a trimeric, tetrameric and/or higher order constructs as described herein.

In one embodiment, the vector is functional in mammalian cells. In a preferred embodiment, the vector is functional in a neural cell. In another embodiment, the vector is functional in a neuron.

In one embodiment, a host cell is provided comprising the polynucleotide, the expression vector or polypeptide as disclosed herein.

Mechanism of Action

As demonstrated in the present disclosure, the polynucleotide of the present disclosure encodes a polypeptide having high affinity towards PDZ domains. As a result of the oligomerization of the polypeptide, to form trimers, tetramers and/or higher order constructs, the affinity towards PDZ domains is significantly increased as compared to monomeric peptide ligands or dimeric peptide ligands.

In one embodiment, when comparing the affinity of a polypeptide of the present disclosure with the affinity of a dimer-forming polypeptide, the affinity of the polypeptide of the present disclosure will be higher than the affinity of the dimer-forming polypeptide. Thus, in one embodiment, the polypeptide as disclosed herein has a higher affinity towards the PDZ domain than the affinity of a polypeptide comprising a first polypeptide part capable of forming a dimer as the highest oligomerization state. In one embodiment, the polypeptide as disclosed herein has a higher affinity towards the PDZ domain than the affinity of a polypeptide comprising GCN4p1 as the first polypeptide part. The affinity may be determined as the Kᵢ such as for example be determined by a fluorescence polarization experiment as disclosed in Examples 4, 5, and 8 of the present disclosures. A lower Kᵢ is equal to a higher affinity. Alternatively, the affinity may be determined by other methods known to the skilled person.

The polypeptide of the present disclosure comprises two polypeptide parts. A first polypeptide part is capable of self-assembling into trimer, tetramer and/or higher order constructs and thereby functions as an oligomerization domain. The higher order constructs may be formed as a coiled coil structure. The second polypeptide part functions as a ligand part which is capable of binding to a PDZ domain. Thus, in one embodiment, the second polypeptide of the present invention binds to a PDZ domain. Binding of the second polypeptide part to the PDZ domain of a given protein may provide inhibition of said protein.

Oligomerization of the polypeptide of the present disclosure functions to position three or more peptide ligands in close proximity, such a conjugating three or more peptide ligands to each other via the oligomerization domain. The peptide ligands of the multimeric construct may then be able to bind PDZ domains of different PDZ domain containing proteins, such as of two proteins, for example of three proteins, such as of four proteins, for example of five proteins, such as of six proteins, for example of seven proteins, thereby forming higher order complexes of PDZ domain containing proteins.

In some embodiment, binding of the polypeptide encoded by the polynucleotide of the present disclosure to a PDZ domain containing protein, results in trimerization of said protein. For example, the polypeptide may bind to PDZ domains of three separate proteins, thereby bringing the three proteins together to form a trimeric complex. In one embodiment, the PDZ domains are inhibited by formation of this trimeric complex.

In one embodiment, the PDZ domain containing protein is PICK1 which is known to be present in a dimer conformation, with dimerization mediated by the BAR domain. It has been reported that dimerization of the dimeric PICK1, providing dimers of dimers, such as tetramers, results in auto-inhibition of the protein function (Karlsen, M. L. et al. 2015). It can thus be hypothesized that binding of the polypeptide of the present disclosure, which is present as a higher order construct, functions by bringing together several PICK1 proteins, thereby leading to the observed effective inhibition of PICK1.

In one embodiment, binding of the polypeptide of the present disclosure to the PDZ domain of PICK1 results in formation of higher oligomeric states of PICK1, such as trimers, tetramers, pentamers, hexamers or heptamers of PICK1. In one embodiment, binding of the polypeptide of the present disclosure to the PDZ domain of PICK1 results in formation of higher oligomeric states of PICK1, such as trimers, tetramers, pentamers, hexamers or heptamers of dimers of PICK1.

In one embodiment, the PDZ domain containing protein is PSD-95. Thus, in one embodiment, formation of higher order complexes of the PDZ domain containing protein does not result in auto-inhibition of the protein. As demonstrated by the present disclosure, the polypeptides of the present disclosure provide highly potent inhibitors of PSD-95. Thus, in one embodiment, it is not a prerequisite for the function of the polypeptide of the present disclosure that the target protein is auto-inhibited upon formation of higher order complexes.

As demonstrated in the present disclosure, binding of the polypeptide of the present disclosure, which is present as a higher order construct, to PSD-95, result in a liquid-liquid phase separation (LLPS). Thus, in one embodiment, the polypeptide of the present disclosure functions by inducing LLPS transition of the PDZ domain containing protein, thereby inhibiting the protein.

Thus, in one embodiment, the polypeptide of the present disclosure inhibits the PDZ domain containing protein, such as inhibits PICK1, PSD-95, nNOS, Shank1, Shank2, Shank3, Syntenin, GRIP, MAGI1, MAGI2, MAGI3, PSD-93, DLG1, SAP-102, ZO-1, Frizzled, PAR3, or PARE, Mint1, or CASK.

In another embodiment, the second polypeptide is capable of inhibiting the protein-protein interaction of a PDZ domain and its respective binding partner.

In one embodiment, the second polypeptide is capable of inhibiting a protein-protein interaction with the PDZ domain, such as the interaction between AMPAR and PICK1, between cytosolic kinases and PICK1, between synaptic scaffold proteins and PICK1, between membrane embedded proteins and PICK1, between NMDAR and PSD-95, between membrane embedded proteins and PSD-95, or between synaptic scaffold proteins and PSD-95.

In one embodiment, the polypeptide has an affinity (K) for the PDZ domain containing protein below 1 μM, such as below 800 nM, such as below 600 nM, such as below 400 nM, such as below 200 nM, such as below 150 nM, such as below 125 nM, such as below 100 nM, such as below 90 nM, such as below 80 nM, such as below 70 nM, such as below 60 nM, such as below 50 nM, such as below 40 nM, such as below 30 nM, such as below 20 nM, such as below 10 nM. Binding affinity (K) may be determined by the method as disclosed in Examples 4 and 5 and 8.

In one embodiment, the polypeptide as disclosed herein has a higher affinity towards the PDZ domain than the affinity of a polypeptide comprising a first polypeptide part capable of forming a dimer as the highest oligomerization state. In one embodiment, the polypeptide as disclosed herein has a higher affinity towards the PDZ domain than the affinity of a polypeptide comprising GCN4p1 as the first polypeptide part.

Diseases and Disorders

AMPARs are usually only permeable to monovalent cations (i.e. Na⁺ and K⁺) due to presence of the GluA2 subunit in the receptor complex. A specific type of plasticity involving strong and sustained depolarization, however, results in a switch to AMPARs, excluding the GluA2 subunit, with increased conductance and Ca²⁺-permeability (CP-AMPARs) in several types of synapses. Since the AMPARs are readily activated, this switch renders the synapse hypersensitive with respect to both Na⁺ and Ca²⁺ calcium influx stimulated by glutamate. This plasticity plays a central pathophysiological role in development of addiction, initially in midbrain dopaminergic neurons and subsequently, as the addiction process progresses, also in medium spiny neurons, where it underlies cocaine craving. A similar process is involved in the development of neuropathic pain, first in the dorsal horn and subsequently and conceivably, also in the neurons in thalamus and sensory cortex. Finally, CP-AMPARs are also expressed in hippocampal neurons after ischemia and as such the process rather appears to be a maladaptive type of plasticity in response to abnormal levels of glutamate in the synapse. Mechanistically, expression of CP-AMPARs involves an initial PICK1 dependent down-regulation of GluA2 containing AMPARs, which is mediated by the interaction between the PICK1 PDZ domain and the C-terminus of the GluA2 subunit of the AMPARs. The downregulation of GluA2 containing AMPARs is in part regulated by phosphorylation of the AMPAR C-terminal regions by cytosolic kinases; these phosphorylations are also regulated by kinase binding to PICK1.

This in turn allows for insertion of GluA2 lacking receptors in the synapse rendering the synapse Ca²⁺-permeable and hypersensitive.

Inhibition of PICK1 can thus prevent PICK1 from down-regulating GluA2 and prevent CP-AMPARs formation thereby preventing a maladaptive type of plasticity in response to abnormal levels of glutamate in the synapse. This in turn can prevent for example neuropathic pain. In one embodiment, the AMPAR is comprised in a cell.

PSD-95 interacts with several proteins including the simultaneous binding of the NMDA-type of ionotropic glutamate receptors and nNOS. NMDA receptors are implicated in neurodegenerative diseases and acute brain injuries, and although antagonists of the NMDA receptor efficiently reduce excitotoxicity by preventing glutamate-mediated ion-flux, they also prevent physiological important processes.

Specific inhibition of Ca²⁺ mediated excitotoxicity, can be obtained by perturbing the intracellular nNOS/PSD-95/NMDA receptor complex using PSD-95 or nNOS inhibitors, resulting in treatment of similar indications as described above for PICK1. Contrary to PICK1, PSD-95 simultaneously binds the NMDA receptor and nNOS via PDZ1 and PDZ2, respectively. Activation of the NMDA receptor causes influx of Ca²⁺, which activates nNOS thereby leading to NO generation. nNOS activation has also been shown to take place upon insertion of CP-AMPARs, through interaction between PSD-95, transmembrane AMPAR auxiliary subunits (TARPS) (Bissen et al 2019), and nNOS (Socodato et al. 2012). Thus, the PSD-95/nNOS interaction mediates a specific association between CP-AMPARs, NMDA receptors and NO production, which can be detrimental for the cells if sustained for a longer period, and is a key facilitator of glutamate-mediated neurotoxicity. Inhibition of the ternary complex of nNOS/PSD-95/NMDA receptor interaction by targeting PSD-95 is known to prevent ischemic brain damage in mice, primates and humans, by impairing the functional link between Ca²⁺ entry and NO production, while the physiological function, such as ion-flux and pro-survival signaling pathways of the NMDA receptor remains intact (Hill et al. 2020).

In general, PDZ-containing proteins are known to play an important role in cancer, from tumor formation to metastasis, especially through canonical interactions of their PDZ domains in signaling pathways. In fact, 145 of 151 PDZ domain proteins have been suggested to be associated with cancers. Validated drug targets include Scribbled, Syntenin and Disheveled.

A large number of PDZ domain-containing proteins are associated with neurological disorders. Among others, regulating synaptic membrane exocytosis protein 1 (RIMS1), partitioning defective 3 homolog B (PARD3B), peripheral plasma membrane protein CASK, and Post synaptic density protein 95 (PSD-95) are associated with neurodevelopmental disorders, which are central nervous system development disorders with different manifestations. Validated drug targets include PSD95, PICK1 and Shank1-3.

The present invention provides a pharmaceutical composition comprising a polynucleotide, an expression vector, a polypeptide and/or a host cell as disclosed herein. In one embodiment, a pharmaceutical composition as disclosed herein is provided for treatment of diseases and/or disorders associated with maladaptive plasticity.

As demonstrated in the examples of the present disclosure, the present disclosure provides polynucleotides for use in treatment of a disease and/or disorder associated with maladaptive plasticity and/or transmission, such as for use in treatment in inflammatory pain, as demonstrated in Example 10.

The present disclosure provides a polynucleotide, an expression vector, a polypeptide, a host cell, and/or a pharmaceutical composition as described herein for use as a medicament. In one embodiment a polynucleotide, an expression vector, a polypeptide, a host cell, and/or a pharmaceutical composition as described herein is provided for use in treatment of a disease and/or disorder associated with maladaptive plasticity and/or transmission.

In diseased states, such as ischemia after stroke, neuropathic pain and addiction, abnormal synaptic stimulation causes maladaptive plasticity leading to hyper-sensitization of glutamatergic synapses through expression of calcium permeable (CP) AMPA-type glutamate receptors (CP-AMPARs).

AMPA-type glutamate receptors (AMPARs) are, in contrast to NMDA-type glutamate receptors (NMDARs), usually only permeable to monovalent cations (i.e. Na+ and K+) due to presence of GluA2 subunits in the tetrameric receptor complex. Plasticity changes in response to a strong and sustained depolarization, however, result in a switch to AMPARs with increased conductance and Ca²⁺ permeability (CP-AMPARs) in several types of synapses and this switch renders the synapse hypersensitive. Mechanistically, expression of CP-AMPARs involves an initial PICK1-dependent down-regulation of GluA2 containing AMPARs, which is mediated by the interaction between the PICK1 PDZ domain and the C-terminus of the GluA2 subunit of the AMPARs. This in turn allows for insertion of GluA2 lacking receptors in the synapse (Slot hypothesis) rendering the synapse Ca²⁺-permeable and hypersensitive.

CP-AMPARs are critically involved in the mediating craving after withdrawal from cocaine self-administration in rats (Conrad et al 2008). PICK1 has been implicated in the expression of CP-AMPAR in the VTA dopaminergic neurons in midbrain and in nucleus accumbens during development of cocaine craving (Luscher et al 2011 and Wolf et al 2010) suggesting PICK1 as a target in cocaine addiction. Thus in one embodiment, administration of a polynucleotide, an expression vector, a polypeptide, a host cell, and/or a pharmaceutical composition as described herein reduces cocaine craving in drug addiction, such as cocaine addiction.

Upregulation of AMPA-type glutamate receptors (AMPARs) in the dorsal horn (DH) neurons causes central sensitization, a specific form of synaptic plasticity in the DH sustainable for a long period of time (Woolf et al 2000 and Ji et al 2003). Moreover, both peripheral inflammatory pain and nerve injury induced pain, cause upregulation of Ca2+-permeable AMPARs (CP-AMPARs) (Vikman et al 2008, Gangadharan et al 2011 and Chen et al 2013). Initial evidence for a role of PICK1 in neuropathic pain came from Garry et al 2003 demonstrating that peptide inhibitors of PICK1 alleviated pain induced by chronic constriction injury (CCI). Subsequently, it was demonstrated the shRNA mediated knock down of PICK1 alleviated complete Freud's adjuvans (CFA) induced inflammatory pain and it was found that PICK1 knock-out mice completely fail to develop pain in response to spinal nerve ligation (SNL) (Wang et al 2011 and Atianjoh et al 2010). Thus, administration of the polynucleotides of the present disclosure may reduce mechanical allodynia in neuropathic pain and/or inflammatory pain.

Both TDP-43 pathology and failure of RNA editing of the AMPA receptor subunit GluA2, are etiology-linked molecular abnormalities that concomitantly occur in the motor neurons of the majority of patients with amyotrophic lateral sclerosis (ALS). Pain symptoms in a mouse model with conditional knock-out of the RNA editing enzyme adenosine deaminase acting on RNA 2 (ADAR2) are relieved by the AMPAR antagonist perampanel, suggesting a likely symptomatic relief by the polynucleotides or polypeptides of the present disclosure.

Given the predicted effect of the polynucleotides or polypeptides of the present disclosure on pain and predicted effect on addiction, we expect also good efficacy of the polynucleotides or polypeptides on patient with comorbidity e.g. pain patients with opioid addiction.

Similar central sensitization is thought to underlie the allodynia in hyperalgesic priming, which serves as an experimental model for lower back pain and migraine (Kandasamy et al 2015). Similarly, the etiology for tinnitus holds several parallels with neuropathic pain including central sensitization (Vanneste et al 2019, Peker et al 2016 and Moller et al 2007).

A role for PICK1 in the surface stabilization/insertion of CP-AMPARs has been described for oxygen-glucose depletion in cultured hippocampal neurons (Clem et al 2010 and Dixon et al 2009). This evokes PICK1 as a putative target in the protection of neural death after ischemic insult.

Loss of PICK1 has been demonstrated to protect neurons in vitro and in vivo against spine loss in response to amyloid beta (Marcotte et al 2018 and Alfonso et al 2014). Consequently, PICK1 is a putative target for symptomatic and perhaps preventive treatment of Alzheimer's disease.

PICK1 interacts and inhibits the E3 ubiquitin ligase Parkin, which is involved in mitophagy. Parkin loss of function is associated with both sporadic and familial Parkinson's disease (PD). As a result, PICK1 KO mice are resistant to 1-methyl-4-phenyl-1,2,3,6-tetrahydropyridine (MPTP)-mediated toxicity (He et al 2018). Consequently, PICK1 is a putative target for symptomatic and perhaps preventive treatment of Parkinson's disease.

Overstimulation of glutamate receptors resulting in excessive intracellular calcium concentrations is a major cause of neuronal cell death in epilepsy. The GluR2 (GluA2) hypothesis states that following a neurological insult such as an epileptic seizure, the AMPA receptor subunit GluR2 protein is downregulated. This increases the likelihood of the formation of GluR2-lacking, calcium-permeable AMPA receptor which might further enhance the toxicity of the neurotransmitter, glutamate (Lorgen et al 2017).

PICK1 is overexpressed in tumor cells as compared to adjacent normal epithelia in breast, lung, gastric, colorectal, and ovarian cancer. As judged by immunostaining breast cancer tissue microarrays, high levels of PICK1 expression correlates with shortened span of overall survival. Accordingly, transfection of MDA-MB-231 cells with PICK1 siRNA decreased cell proliferation and colony formation in vitro and inhibited tumorigenicity in nude mice (Zhang et al 2010). Consequently, PICK1 is a putative target for cancer treatment and prognostics.

In one embodiment, a polynucleotide, an expression vector, a polypeptide, a host cell, and/or a composition as disclosed herein is provided for use as a medicament.

The present invention provides the polynucleotide, the expression vector, the polypeptide, the cell, and/or the composition as described herein for use in treatment of a disease and/or disorder associated with maladaptive plasticity and/or transmission.

In one embodiment, a polynucleotide, an expression vector, a polypeptide, a host cell, and/or a composition as disclosed herein is provided for the manufacture of a medicament for the treatment of diseases and/or disorders associated with maladaptive plasticity and/or transmission.

In one embodiment, a method of treatment or prevention of a disease and/or disorder associated with maladaptive plasticity and/or transmission in a subject in need thereof is provided, the method comprising administering a therapeutically effective amount of a polynucleotide, an expression vector, a polypeptide, a host cell, and/or a composition as disclosed herein to said subject.

In one embodiment, the disease or disorder associated with maladaptive plasticity is pain, drug addiction, amyotrophic lateral sclerosis, epilepsy, tinnitus, migraine, cancer, ischemia, Alzheimer's disease, and/or Parkinson's disease.

In one embodiment, the disease or disorder associated with maladaptive plasticity is pain, such as neuropathic pain. The pain can be inflammatory pain or neuropathic pain. The pain, to be treated, may be chronic pain, which may be chronic neuropathic pain or chronic inflammatory pain. The neuropathic pain may be induced by damage to the peripheral or central nervous system as a result of traumatic injury, surgery, or diseases such as diabetes, autoimmune disorders, or amputation. The neuropathic pain may be induced by treatment with chemotherapy. Where pain persists, the condition is chronic neuropathic pain. Chronic inflammatory pain may be induced by inflammation after nerve injury, as well as being initiated by inflammation induced by alien matter, where mediators released by immune cells cause a sensitization of pain pathways, i.e. a ‘wind up’ of sensory neurons located in the spinal cord. Thus, an effective analgesic drug must be able to reach spinal cord tissue and find its target, in this case PICK1, in order to have a pain-relieving effect. Thereby, the compounds must be able to pass the blood-brain barrier and/or blood-spinal cord barrier to be able to reach spinal cord tissue.

In one embodiment, the disease or disorder associated with maladaptive plasticity is drug addiction, such as cocaine addiction, opioid addiction, or morphine addiction.

In one embodiment, the disease or disorder associated with maladaptive plasticity is cancer such as breast cancer, for example histological grade, lymph node metastasis, Her-2/neu-positivity, and triple-negative basal-like breast cancer.

In one embodiment, the disease or disorder associated with maladaptive plasticity is amyotrophic lateral sclerosis.

In one embodiment, the disease or disorder associated with maladaptive plasticity is epilepsy.

In one embodiment, the disease or disorder associated with maladaptive plasticity is tinnitus.

In one embodiment, the disease or disorder associated with maladaptive plasticity is migraine.

In one embodiment, the disease or disorder associated with maladaptive plasticity is stroke or ischemia.

In one embodiment, the disease or disorder associated with maladaptive plasticity is Alzheimer's disease.

In one embodiment, the disease or disorder associated with maladaptive plasticity is Parkinson's disease.

In yet another embodiment, the compound as disclosed herein is for use in the prophylaxis and/or treatment of head injury.

In yet another embodiment, the compound as disclosed herein is for use in the prophylaxis and/or treatment and/or diagnosis of cancer, such as breast cancer.

Subjects at risk or presently suffering from the above disorders and diseases may be given either prophylactic treatment to reduce the risk of the disorder or disease onset or therapeutic treatment following the disorder or disease onset. The subject may be a mammalian or human patient.

Administration

Conventional methods, known to those of ordinary skill in the art of medicine, can be used to administer the polynucleotide, the vector, the host cell or the polypeptide of the present disclosure to the subject or patient.

The polynucleotide, the vector, the host cell or the polypeptide of the present disclosure may be administered alone, or in combination with other therapeutic agents or interventions.

In one embodiment, the pharmaceutical composition of the present disclosure is administered prior to observing symptoms of a given indication, such as administered prior to injury for the treatment of pain.

In one embodiment, the pharmaceutical composition of the present disclosure is administered after observing symptoms of a given indication, such as administered after injury for the treatment of pain.

Items
1. A polynucleotide comprising a sequence encoding a polypeptide comprising:
   a) a first polypeptide part comprising or consisting of an amino acid sequence capable of forming a trimer, tetramer and/or higher order oligomer; and
   b) a second polypeptide part comprising or consisting of an amino acid sequence selected from the group consisting of Class I PDZ domains binding motifs (PBM), Class II PBM and Class III PBM, wherein the first and the second polypeptides are optionally operably linked via a linker.
2. A polynucleotide comprising a sequence encoding a polypeptide comprising:
   a) a first polypeptide part comprising or consisting of an amino acid sequence selected from the group consisting of GCN4p1(NQ) (SEQ ID NO: 67), GCN4p1(LI) (SEQ ID NO: 68), GCN4p1(ILI) (SEQ ID NO: 147), CC-Tet (SEQ ID NO: 69), cc-Hex2 (SEQ ID NO: 70), ATF7-pII (SEQ ID NO: 154), ATF2-pII (SEQ ID NO:159), NRP-pII (SEQ ID NO:160), PIX-pII (SEQ ID NO:161), HLF-pll (SEQ ID NO:162), DBP-pII (SEQ ID NO:163), TEF-pII (SEQ ID NO:164), NRBI-pII (SEQ ID NO:165), CREB4-pII (SEQ ID NO:166), CREBH-pII (SEQ ID NO:167), MAT2-pII (SEQ ID NO:168); and
   b) a second polypeptide part comprising or consisting of an amino acid sequence selected from the group consisting of Class I PDZ domains binding motifs (PBM), Class II PBM and Class III PBM.
3. The polypeptide according to item 2, wherein the first polypeptide part comprises or consists of an amino acid sequence selected from the group consisting of GCN4p1(LI) (SEQ ID NO: 68) and GCN4p1(ILI) (SEQ ID NO: 147).
4. The polynucleotide according to any one of the preceding items, wherein the second polypeptide part is consisting of or comprising a Class I PBM comprising an amino acid sequence of Σ-¥-ψ, a Class II PBM comprising an amino acid sequence of ψ-¥-ψ, or a Class II PBM comprising an amino acid sequence of ϕ-¥-ψ, wherein
   Σ is Thr, Cys or Ser;
   ¥ is any proteinogenic amino acid;
   ψ is any hydrophobic amino acid; and
   ϕ is Asp or Glu.
5. The polynucleotide according to any one of the preceding items, wherein the second polypeptide part comprises a Class I PBM comprising or consisting of a sequence of Σ-¥-ψ, wherein is Thr, Cys or Ser, is any proteinogenic amino acid and ψ is any hydrophobic amino acid, such as comprising or consisting of a sequence selected from the group consisting of IETDV, RRTTPV, and YKQTSV.
6. The polynucleotide according to any one of the preceding items, wherein the second polypeptide part comprises a Class II PBM comprising or consisting of a sequence ψ-¥-ψ, wherein is any proteinogenic amino acid and ψ is any hydrophobic amino acid, such as comprising or consisting of the sequence HWLKV.
7. The polynucleotide according to any one of the preceding items, wherein the second polypeptide part comprises a Class III PBM comprising or consisting of a sequence of ϕ-¥-ψ, wherein ϕ is Asp or Glu, ¥ is any proteinogenic amino acid and ψ is any hydrophobic amino acid, such as comprising or consisting of a sequence selected from the group consisting of WGESV, KVDSV, GKDYV, RKDYV, TAEMF and QEDGA.
8. The polynucleotide according to any one of the preceding items, wherein the second polypeptide part is selected from the group consisting of HWLKV, IETDV, RRTTPV, and YKQTSV.
9. The polynucleotide according to any one of the preceding items, wherein the second polypeptide part is comprising or consisting of an amino acid sequence of the general formula X₁X₂X₃X₄X₅X₆;
   wherein
   X₁ is Y, R or is absent;
   X₂ is R, K, I or is absent;
   X₃ is T, E, Q; or is absent;
   X₄ is T;
   X₅ is D, S or P; and
   X₆ is V.
10. The polynucleotide according to any one of the preceding items, wherein the second polypeptide part is comprising or consisting of an amino acid sequence of the general formula: X₁X₂X₃X₄X₅;
   wherein
   X₁ is H, N, F, or T, or is absent;
   X₂ is W, S, E, or Y; or is absent;
   X₃ is L, V, or I;
   X₄ is K, I, or R; and
   X₅ is V.
11. The polynucleotide according to item 7, wherein:
   X₁ is N, F, or T, or is absent;
   X₂ is S, E, or Y; or is absent;
   X₃ is V, L or I;
   X₄ is I or R; and
   X₅ is V.
12. The polynucleotide according to item 7, wherein:
   X₁ is N or T, or is absent;
   X₂ is S, E, or Y; or is absent;
   X₃ is V or I;
   X₄ is I or R; and
   X₅ is V.
13. The polynucleotide according to item 7, wherein:
   X₁ is N or F, or is absent;
   X₂ is S, E, or Y; or is absent;
   X₃ is V or I;
   X₄ is I or R; and
   X₅ is V.
14. The polynucleotide according to any one of the preceding items, wherein the second polypeptide part is selected from the group consisting of HWLKV, FEIRV, NSIIV, NSVRV, NSLRV, NSIRV, NYIIV, NYIRV, TSIRV, YIIV, SVRV, EIRV, LRV, IIV, VRV, and IRV.
15. The polynucleotide according to any one of the preceding items, wherein the second polypeptide part is selected from the group consisting of HWLKV, NSVRV, NSLRV, NSIRV, TSIRV, EIRV, YIIV, IIV, and IRV.
16. The polynucleotide according to any one of the preceding items, wherein the second polypeptide part is selected from the group consisting of NSVRV, NSLRV, NSIRV, TSIRV, EIRV, YIIV, IIV, and IRV.
17. The polynucleotide according to any one of the preceding items, wherein the second polypeptide part is selected from the group consisting of HWLKV, FEIRV, NSIIV, NSVRV, NSLRV, NSIRV, YIIV, SVRV, VRV, and LRV.
18. The polynucleotide according to any one of the preceding items, wherein the second polypeptide part is selected from the group consisting of FEIRV, NSIIV, NSVRV, NSLRV, NSIRV, YIIV, SVRV, VRV, and LRV.
19. The polynucleotide according to any one of the preceding items, wherein the second polypeptide part is HWLKV.
20. The polynucleotide according to any one of the preceding items, wherein the first polypeptide part is selected from the group consisting of GCN4p1(NQ) (SEQ ID NO: 67), GCN4p1(LI) (SEQ ID NO: 68), GCN4p1(ILI) (SEQ ID NO: 147), CC-Tet (SEQ ID NO: 69), cc-Hex2 (SEQ ID NO: 70), ATF7-pII (SEQ ID NO: 154), ATF2-pll (SEQ ID NO:159), NRP-pII (SEQ ID NO:160), PIX-pII (SEQ ID NO:161), HLF-pll (SEQ ID NO:162), DBP-pII (SEQ ID NO:163), TEF-pII (SEQ ID NO:164), NRBI-pll (SEQ ID NO:165), CREB4-pII (SEQ ID NO:166), CREBH-pII (SEQ ID NO:167), and MAT2-pII (SEQ ID NO:168).
21. The polynucleotide according to any one of the preceding items, wherein the first polypeptide part is selected from the group consisting of GCN4p1(LI) (SEQ ID NO: 68) and GCN4p1(ILI) (SEQ ID NO: 147).
22. The polynucleotide according to any one of the preceding items, wherein the first polypeptide part is selected from the group consisting of GCN4p1(NQ), GCN4p1(LI), CC-Tet, and CC-Hex2.
23. The polynucleotide according to any one of the preceding items, wherein the first polypeptide part is GCN4p1(NQ) or GCN4p1(LI).
24. The polynucleotide according to any one of the preceding items, wherein the first polypeptide part is an alpha helix, such as an amphipathic helix.
25. The polynucleotide according to any one of the preceding items, wherein the first polypeptide part is capable of forming a coiled coil, such as a coiled coil comprising three polypeptides, for example comprising four polypeptides, such as comprising five polypeptides, for example comprising six polypeptides, such as comprising seven polypeptides.
26. The polynucleotide according to any one of the preceding items, wherein the first polypeptide part is positioned N-terminal to the second polypeptide part.
27. The polynucleotide according to any one of the preceding items, wherein the optional linker is a peptide linker, such as a glycine serine (GS) linker.
28. The polynucleotide according to any one of the preceding items, wherein the optional linker is a glycine serine linker selected from the group consisting of GGS (gLinker2, GS2), GGGS (gLinker3, GS3, SEQ ID NO: 71), GGGGS (glinker4, GS4, SEQ ID NO: 72), GGGGSG (gLinker5, GS5, SEQ ID NO: 73), GGGGSGG (gLinker6, GS6, SEQ ID NO: 74).
29. The polynucleotide according to any one of the preceding items, wherein the optional linker is GGGGS (glinker4, GS4, SEQ ID NO: 72).
30. The polynucleotide according to any one of the preceding items, wherein the polypeptide linker comprises 1 to 12 repeats of the GGGGS moieties.
31. The polynucleotide according to any one of the preceding items, wherein the first polypeptide part is selected from the group consisting of: SEQ ID NO: 67, 68, 69, 70, 154, and any one of 159-168, the linker is selected from GGS, and any one of SEQ ID NO: 71-74, and the second polypeptide is selected from any one of SEQ ID NO: 5-64 or IIV, IRV, VIV, VRV, and LRV.
32. The polynucleotide according to any one of the preceding items, wherein the first polypeptide part is selected from the group consisting of: SEQ ID NO: 67, 68, 69, 70, and 154, the linker is SEQ ID NO: 72, and the second polypeptide is selected from any one of SEQ ID NO: 5-64 or IIV, IRV, VIV, VRV, and LRV.
33. The polynucleotide according to any one of the preceding items, wherein the polypeptide is selected from the group consisting of

(SEQ ID NO: 75)
RMKQLEDKVEELLSKQYHLENEVARLKKLVGGGGSHWLKV,
(SEQ ID NO: 76)
RMKQLEDKVEELLSKQYHLENEVARLKKLVGGGGSNSIRV,
(SEQ ID NO: 77)
RMKQLEDKVEELLSKQYHLENEVARLKKLVGGGGSIETDV,
(SEQ ID NO: 78)
RMKQLEDKVEELLSKQYHLENEVARLKKLVGGGGSRRTTPV,
(SEQ ID NO: 79)
RMKQLEDKVEELLSKQYHLENEVARLKKLVGGGGSYKQTSV,
(SEQ ID NO: 80)
RMKQLEDKVEELLSKQYHLENEVARLKKLVGGGGSWGESV,
(SEQ ID NO: 81)
RIKQIEDKIEEILSKIYHIENEIARIKKLIGGGGSHWLKV,
(SEQ ID NO: 82)
RIKQIEDKIEEILSKIYHIENEIARIKKLIGGGGSNSIRV,
(SEQ ID NO: 83)
RIKQIEDKIEEILSKIYHIENEIARIKKLIGGGGSIETDV,
(SEQ ID NO: 84)
RIKQIEDKIEEILSKIYHIENEIARIKKLIGGGGSRRTTPV,
(SEQ ID NO: 85)
RIKQIEDKIEEILSKIYHIENEIARIKKLIGGGGSYKQTSV,
(SEQ ID NO: 86)
RIKQIEDKIEEILSKIYHIENEIARIKKLIGGGGSWGESV,
(SEQ ID NO: 87)
GELAAIKQELAAIKKELAAIKWELAAIKQGGGGSHWLKV,
(SEQ ID NO: 88)
GELAAIKQELAAIKKELAAIKWELAAIKQGGGGSNSIRV,
(SEQ ID NO: 89)
GELAAIKQELAAIKKELAAIKWELAAIKQGGGGSIETDV,
(SEQ ID NO: 90)
GELAAIKQELAAIKKELAAIKWELAAIKQGGGGSRRTTPV,
(SEQ ID NO: 91)
GELAAIKQELAAIKKELAAIKWELAAIKQGGGGSYKQTSV,
(SEQ ID NO: 92)
GELAAIKQELAAIKKELAAIKWELAAIKQGGGGSWGESV,
(SEQ ID NO: 93)
GEIAKSLKEIAKSLKEIAWSLKEIAKSLKGGGGSHWLKV,
(SEQ ID NO: 94)
GEIAKSLKEIAKSLKEIAWSLKEIAKSLKGGGGSNSIRV,
(SEQ ID NO: 95)
GEIAKSLKEIAKSLKEIAWSLKEIAKSLKGGGGSIETDV,
(SEQ ID NO: 96)
GEIAKSLKEIAKSLKEIAWSLKEIAKSLKGGGGSRRTTPV,
(SEQ ID NO: 97)
GEIAKSLKEIAKSLKEIAWSLKEIAKSLKGGGGSYKQTSV,
(SEQ ID NO: 98)
GEIAKSLKEIAKSLKEIAWSLKEIAKSLKGGGGSWGESV,
(SEQ ID NO: 144)
RIKQIEDKIEEILSKIYHIENEIARIKKLIGGGGSNSVRV,
(SEQ ID NO: 146)
GEIAKSLKEIAKSLKEIAWSLKEIAKSLKGGGGSNSVRV,
(SEQ ID NO: 148)
RMKQIEDKLEEILSKLYHIENELARIKKLLGGGGSHWLKV,
(SEQ ID NO: 149)
RMKQIEDKLEEILSKLYHIENELARIKKLLGGGGSNSVRV,
(SEQ ID NO: 150)
RMKQIEDKLEEILSKLYHIENELARIKKLLGGGGSIETDV,
(SEQ ID NO: 151)
RMKQIEDKLEEILSKLYHIENELARIKKLLGGGGSRRTTPV,
(SEQ ID NO: 152)
RMKQIEDKLEEILSKLYHIENELARIKKLLGGGGSWGESV,
(SEQ ID NO: 194)
VSSIEKKIEEITSQIIQISNEITLIRNEIAQIKQGGGGSHWLKV,
and
(SEQ ID NO: 195)
VSSIEKKIEEITSQIIQISNEITLIRNEIAQIKQGGGGSIETDV.

34. The polynucleotide according to any one of the preceding items, wherein the polynucleotide comprises
   a. A nucleotide sequence selected from the group consisting of SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO:169, SEQ ID NO:171, SEQ ID NO:172, SEQ ID NO:174, SEQ ID NO:175, SEQ ID NO:176, SEQ ID NO:177, SEQ ID NO:178, SEQ ID NO: 192, and SEQ ID NO: 193, or
   b. a sequence variant of a nucleotide sequence selected from the group of a), wherein the sequence variant has at least 70% sequence identity to said nucleotide sequence, such as at least 75% sequence identity, for example at least 80% sequence identity, such as at least 85% sequence identity, for example at least 90% sequence identity, such as at least 95% sequence identity, for example at least 96% sequence identity, such as at least 97% sequence identity, for example at least 98% sequence identity, such as at least 99% sequence identity to said nucleotide sequence.
35. The polynucleotide according to any one of the preceding items, wherein the polynucleotide comprises a sequence selected from the group consisting of SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO:169, SEQ ID NO:171, SEQ ID NO:172, SEQ ID NO:174, SEQ ID NO:175, SEQ ID NO:176, SEQ ID NO:177, SEQ ID NO:178, SEQ ID NO: 192, and SEQ ID NO: 193.
36. The polynucleotide according to any one of the preceding items, wherein the polynucleotide comprises a sequence selected from the group consisting of SEQ ID NO: 126, SEQ ID NO: 127, SEQ ID NO: 128, SEQ ID NO: 129, SEQ ID NO: 130, SEQ ID NO: 131, SEQ ID NO: 169, SEQ ID NO: 174, SEQ ID NO: 175, SEQ ID NO: 176, SEQ ID NO: 177, and SEQ ID NO: 178.
37. The polynucleotide according to any one of the preceding items, wherein the polynucleotide comprises a sequence selected from the group consisting of SEQ ID NO: 120, SEQ ID NO: 121, SEQ ID NO: 126, SEQ ID NO: 127, SEQ ID NO: 132, SEQ ID NO: 133, SEQ ID NO: 138, SEQ ID NO: 139, SEQ ID NO: 169, SEQ ID NO: 171, SEQ ID NO: 172, and SEQ ID NO: 174, SEQ ID NO: 175, and SEQ ID NO: 192.
38. The polynucleotide according to any one of the preceding items, wherein the polynucleotide comprises a sequence selected from the group consisting of SEQ ID NO: 120, SEQ ID NO: 126, SEQ ID NO: 132, SEQ ID NO: 138, and SEQ ID NO: 174.
39. The polynucleotide according to any one of the preceding items, wherein the polynucleotide comprises a sequence selected from the group consisting of SEQ ID NO: 126 and SEQ ID NO: 174.
40. The polynucleotide according to any one of the preceding items, wherein the polynucleotide comprises a sequence selected from the group consisting of SEQ ID NO: 122, SEQ ID NO: 123, SEQ ID NO: 128, SEQ ID NO: 129, SEQ ID NO: 134, SEQ ID NO: 135, SEQ ID NO: 140, SEQ ID NO: 141, SEQ ID NO: 176, SEQ ID NO: 177, and SEQ ID NO: 193.
41. The polynucleotide according to any one of the preceding items, wherein the polynucleotide comprises a sequence selected from the group consisting of SEQ ID NO: 122, SEQ ID NO: 128, SEQ ID NO: 134, SEQ ID NO: 140, SEQ ID NO: 176, and SEQ ID NO: 193.
42. The polynucleotide according to any one of the preceding items, wherein the polynucleotide comprises a sequence selected from the group consisting of SEQ ID NO: 128 and SEQ ID NO: 176.
43. The polynucleotide according to any one of the preceding items, wherein the polynucleotide comprises a sequence selected from the group consisting of SEQ ID NO: 125, SEQ ID NO: 131, SEQ ID NO: 137, SEQ ID NO: 143, and SEQ ID NO: 178.
44. The polynucleotide according to any one of the preceding items, wherein the nucleotide sequence is codon optimized for expression in human beings.
45. The polynucleotide according to any one of the preceding items, wherein the polypeptide further comprises a cell penetrating peptide (CPP).
46. The polynucleotide according to any one of the preceding items, wherein the CPP is connected to the polypeptide via a linker, such as a polypeptide linker, such as a glycine serine linker.
47. The polynucleotide according to any one of the preceding items, wherein the CPP is positioned N-terminal to the first and the second polypeptide parts.
48. The polynucleotide according to any one of the preceding items, wherein the CPP is selected from the group consisting of TAT, polyarginine, TP10, MAP and PNT.
49. The polynucleotide according to any of the preceding items, wherein the first polypeptide further comprises a tag.
50. The polynucleotide according to any one of the preceding items, wherein the tag is conjugated to the N-terminus of the polypeptide.
51. The polynucleotide according to any one of the preceding items, wherein the tag is an HA-tag.
52. The polynucleotide according to any one of the preceding items, wherein the tag is a Myc-tag or His-tag.
53. The polynucleotide according to any one of the preceding items, wherein the tag is a Biotin tag.
54. The polynucleotide according to any one of the preceding items, wherein the tag is operably conjugated to the polypeptide by a GS linker or a 6-aminohexanoic acid (Ahx) linker.
55. The polynucleotide according to any of the preceding items, wherein the second polypeptide is capable of binding to a PDZ domain.
56. The polynucleotide according to any of the preceding items, wherein the second polypeptide is capable of inhibiting a protein-protein interaction with the PDZ domain, such as the interaction between AMPAR and PICK1, between cytosolic kinases and PICK1, between synaptic scaffold proteins and PICK1, between membrane embedded proteins and PICK1, between NMDAR and PSD-95, between membrane embedded proteins and PSD-95, between enzymes and PSD-95, or between synaptic scaffold proteins and PSD-95.
57. The polynucleotide according to any of the preceding items, wherein the second polypeptide inhibits a PDZ domain containing protein, such as inhibits PICK1, PSD-95, nNOS, Shank1, Shank2, Shank3, Syntenin, GRIP, MAGI1, MAGI2, MAGI3, PSD-93, DLG1, ZO-1, Frizzled, PAR3, or PARE, Mint1, or CASK.
58. The polynucleotide according to any of the preceding items, wherein polypeptide has a Ki for the PDZ domain below 1 μM, such as below 800 nM, such as below 600 nM, such as below 400 nM, such as below 200 nM, such as below 150 nM, such as below 125 nM, such as below 100 nM, such as below 90 nM, such as below 80 nM, such as below 70 nM, such as below 60 nM, such as below 50 nM, such as below 40 nM, such as below 30 nM, such as below 20 nM, such as below 10 nM.
59. The polynucleotide according to any of the preceding items, wherein the second polypeptide is capable of binding to the PDZ domain of PICK1.
60. The polynucleotide according to any of the preceding items, wherein binding of the second polypeptide part to the PDZ domain of PICK1 result in formation of higher oligomeric states of PICK1, such as trimers, tetramers, pentamers, hexamers, heptamers or octamers of PICK1.
61. The polynucleotide according to any one of the preceding items further comprising a promoter sequence.
62. The polynucleotide according to any of the preceding items, wherein said promoter is a human Synapsin1 promoter.
63. The polynucleotide according to any of the preceding items, wherein said promoter is a constitutive promoter.
64. The polynucleotide according to any of the preceding items, wherein said constitutively active promoter is selected from the group consisting of CAG, CBA, CMV, human UbiC, RSV, EF-1alpha, NSE, SV40, Mt1.
65. The polynucleotide according to any of the preceding items, wherein said promoter is an inducible promoter.
66. The polynucleotide according to any of the preceding items, wherein said inducible promoter is selected from the group consisting of Tet-On, Tet-Off, Mo-MLV-LTR, Mx1, progesterone, RU486 and Rapamycin-inducible promoter.
67. The polynucleotide according to any of the preceding items, wherein said promoter is an activity-dependent promoter.
68. The polynucleotide according to any of the preceding items, wherein said activity-dependent promoter is selected from the group consisting of cFos, Arc, Npas4, Egr1 promoters.
69. An expression vector comprising the polynucleotide according to any of the preceding items.
70. The expression vector according to any of the preceding items, wherein the vector is selected from the group consisting of RNA based vectors, DNA based vectors, lipid based vectors, polymer based vectors and colloidal gold particles.
71. The expression vector according to any of the preceding items, wherein the vector is a viral vector.
72. The expression vector according to any of the preceding items, wherein the viral vector is a virally derived DNA vector or a virally derived RNA vector.
73. The expression vector according to any of the preceding items, wherein the vector is selected from the group consisting of adenoviruses, recombinant adeno-associated viruses (rAAV), retroviruses, lentiviruses, adeno-associated viruses, herpesviruses, vaccinia viruses, foamy viruses, cytomegaloviruses, Semliki forest virus, poxviruses, RNA virus vector and DNA virus vector.
74. The expression vector according to any of the preceding items, wherein the expression vector is an adeno associated vector (AAV).
75. The expression vector according to any of the preceding items, wherein the adeno associated vector (AAV) is an AAV1 vector, an AAV2 vector, an AAV5, an AAV8 or an AAV9 vector.
76. The expression vector according to any of the preceding items, wherein the plasmid of the AAV1 vector is packaged in an AAV capsid other than an AAV1 capsid.
77. The expression vector according to any of the preceding items, wherein the plasmid of the AAV2 vector is packaged in an AAV capsid other than an AAV2 capsid.
78. The expression vector according to any of the preceding items, wherein the plasmid of the AAV8 vector is packaged in an AAV capsid other than an AAV8 capsid.
79. The expression vector according to any of the preceding items, wherein the plasmid of the AAV9 vector is packaged in an AAV capsid other than an AAV9 capsid.
80. The expression vector according to any of the preceding items, wherein said vector is comprising a polynucleotide sequence encoding the polypeptide according to any one of the preceding items.
81. The expression vector according to any of the preceding items, wherein said vector is functional in mammalian cells.
82. The expression vector according to any of the preceding items, wherein the mammalian cell is a neural cell.
83. The expression vector according to any of the preceding items, wherein the neural cell is a neuron.
84. A polypeptide encoded by the polynucleotide, or the expression vector according to any one of the preceding items.
85. A host cell comprising the polynucleotide, the expression vector or polypeptide according to any one of the preceding items.
86. A pharmaceutical composition comprising the polynucleotide, the expression vector, the host cell or the polypeptide according to any one of the preceding items.
87. The polynucleotide, the expression vector, the polypeptide, the host cell, and/or the pharmaceutical composition according to any one of the preceding items, for use as a medicament.
88. The polynucleotide, the expression vector, the polypeptide, the host cell, and/or the pharmaceutical composition according to any one of the preceding items for use in the prophylaxis and/or treatment of a disease and/or disorder associated with maladaptive plasticity.
89. A method of treatment or prevention of a disease and/or disorder associated with maladaptive plasticity in a subject in need thereof, comprising administering a therapeutically effective amount of the polynucleotide, the expression vector, the polypeptide, the host cell, and/or the pharmaceutical composition according to any one of the preceding items.
90. Use of the polynucleotide, the expression vector, the polypeptide, the host cell, and/or the pharmaceutical composition according to any one of the preceding items, for the manufacture of a medicament for the prophylaxis and/or treatment of a disease and/or disorder associated with maladaptive plasticity.
91. The polynucleotide, the expression vector, the polypeptide, the host cell, and/or the pharmaceutical composition for use according to any of items 87-88, the method according to item 89 or the use according to item 90, wherein the disease or disorder associated with maladaptive plasticity is pain, drug addiction, amyotrophic lateral sclerosis, epilepsy, tinnitus, migraine, cancer, ischemia, Alzheimer's disease, and/or Parkinson's disease.
92. The polynucleotide, the expression vector, the polypeptide, the cell, and/or the pharmaceutical composition for use according to any of items 87-88, the method according to item 89 or the use according to item 90, wherein the disease or disorder associated with maladaptive plasticity is pain, such as inflammatory pain or neuropathic pain.
93. The polynucleotide, the expression vector, the polypeptide, the cell, and/or the pharmaceutical composition for use according to any of items 87-88, the method according to item 89 or the use according to item 90, wherein the disease or disorder associated with maladaptive plasticity is drug addiction, such as cocaine addiction, opioid addiction, or morphine addiction.
94. The polynucleotide, the expression vector, the polypeptide, the cell, and/or the pharmaceutical composition for use according to any of items 87-88, the method according to item 89 or the use according to item 90, wherein the disease or disorder associated with maladaptive plasticity is cancer such as breast cancer, for example histological grade, lymph node metastasis, Her-2/neu-positivity, and triple-negative basal-like breast cancer.
95. The polynucleotide, the expression vector, the polypeptide, the cell, and/or the pharmaceutical composition for use according to any of items 87-88, the method according to item 89 or the use according to item 90, wherein the disease or disorder associated with maladaptive plasticity is amyotrophic lateral sclerosis.
96. The polynucleotide, the expression vector, the polypeptide, the cell, and/or the pharmaceutical composition for use according to any of items 87-88, the method according to item 89 or the use according to item 90, wherein the disease or disorder associated with maladaptive plasticity is epilepsy.
97. The polynucleotide, the expression vector, the polypeptide, the cell, and/or the pharmaceutical composition for use according to any of items 87-88, the method according to item 89 or the use according to item 90, wherein the disease or disorder associated with maladaptive plasticity is tinnitus.
98. The polynucleotide, the expression vector, the polypeptide, the cell, and/or the pharmaceutical composition for use according to any of items 87-88, the method according to item 89 or the use according to item 90, wherein the disease or disorder associated with maladaptive plasticity is migraine.
99. The polynucleotide, the expression vector, the polypeptide, the cell, and/or the pharmaceutical composition for use according to any of items 87-88, the method according to item 89 or the use according to item 90, wherein the disease or disorder associated with maladaptive plasticity is ischemia.
100. The polynucleotide, the expression vector, the polypeptide, the cell, and/or the pharmaceutical composition for use according to any of items 87-88, the method according to item 89 or the use according to item 90, wherein the disease or disorder associated with maladaptive plasticity is Alzheimer's disease.
101. The polynucleotide, the expression vector, the polypeptide, the cell, and/or the pharmaceutical composition for use according to any of items 87-88, the method according to item 89 or the use according to item 90, wherein the disease or disorder associated with maladaptive plasticity is Parkinson's disease.
102. The polypeptide according to any one of the preceding items, wherein the polypeptide has a higher affinity and/or lower Kᵢ towards the PDZ domain than the affinity of a polypeptide comprising a first polypeptide part capable of forming a dimer as the highest oligomerization state.
103. The polypeptide according to any one of the preceding items, wherein the polypeptide has a higher affinity and/or lower Kᵢ towards the PDZ domain than the affinity of a polypeptide comprising GCN4p1 as the first polypeptide part.

### EXAMPLES

The following figures and examples are provided below to illustrate the present invention. They are intended to be illustrative and are not to be construed as limiting in any way.

### Example 1: PICK1 Expression and Purification

Full length rat PICK1 (pET41) was prepared as described earlier (Madsen et al. 2005). In brief, PICK1 was expressed in BL21-DE3-pLysS cells and grown at 37° C., induced at OD₆₀₀=0.6 with 1 mM Isopropyl β-D-1-thiogalactopyranoside (IPTG) and grown 16 hrs at 20° C. Cultures were harvested and re-suspended in 50 mM trisaminomethane (Tris), 125 mM NaCl, 2 mM Dithiothreitol (DTT, Sigma), 1% Triton X-100 (Sigma), 20 μg/mL DNAse 1 and ½ a tablet cOmplete protease inhibitor cocktail (Roche) pr. 1 L culture. The re-suspended pellets were frozen at −80° C. for later purification. The lysate was cleared by centrifugation (36,000×g for 30 min at 4° C.), and the supernatant was incubated with Glutathione-Sepharose 4B beads (GE Healthcare) for 2 hrs at 4° C. under gentle rotation and then centrifuged at 4,000×g for 5 min. The supernatant was removed and the beads were washed twice in 35 mL 50 mM Tris, 125 mM NaCl, 2 mM DTT and 0.01% Triton-X100. The beads were transferred to PD-10 Bio-Spin® Chromatography columns (Bio-Rad) and washed with an additional 3 column volumes. Each column was sealed and 0.075 U/μL, Novagen® was added for cleavage 0/N at 4° C. under gentle rotation. PICK1 was eluted on ice and absorption at 280 nm was measured on TECAN plate reader or on a NanoDrop3000. The protein concentration was determined using lambert beers law (A=εcl), εA₂₈₀PICK1=32320 (cm*mol/L)⁻¹.

### Example 2: Protein Expression and Purification of FL-PSD-95

E. Coli cultures (BL21-DE3-pLysS) transformed with a TRX-6xHis-hPSD95 1-724 encoding plasmid (pET-MG-3C) (Zeng et al. 2016), was inoculated in Lysogeny broth (LB) media supplemented with ampicillin and chloramphenicol overnight and transferred into LB medium supplemented with ampicillin and chloramphenicol and grown at 37° C. until OD₆₀₀=0.6. Protein expression was induced with 1 mM Isopropyl β-D-1-thiogalactopyranoside (IPTG) and grown for 8-16 hrs at 16-20° C. Bacteria were harvested and frozen at −80° C. Pellet was thawed and resuspended in 50 mM Tris (pH 8.0), 300 mM NaCl, 1 mM TCEP, 20 μg/μl DNAse, 1 tablet of cOmpete Protease inhibitor pr. 1 L culture. Resuspended bacteria was sonicated for 2 minutes to induce lysis and lysates were cleared by centrifugation at 30.000 g for 20 min. The supernatant was collected and run through to a 5m1 HisTrap HP column and column was washed with 50 mM Tris (pH 8.0), 300 mM NaCl, 10 mM Imidazole, 1 mM TCEP. Bound protein was eluted using a linear gradient from 10-500 mM Imidazole in 50 mM Tris (pH 8.0), 300 mM NaCl, 1 mM TCEP. Protein containing fractions were pooled and purified further using a Superdex 200 pg 1.6/600 Size exclusion column equilibrated in 50 mM Tris (pH 8.0), 300 mM NaCl, 10 mM EDTA, 1 mM TCEP. Protein purity was validated to be at least above 90% using SDS-PAGE, UPLC and LC-MS. Absorption at 280 nm was measured on TECAN plate reader or on a NanoDrop3000. The protein concentration was determined using lambert beers law (A=εcl), εA₂₈₀PSD-95=80220 (cm*mol/L)⁻¹.

### Example 3: Oligomer Formation of GCN4p1 Variants

To test the oligomeric nature of specific GCN4p1 variants we conducted size exclusion chromatography (SEC) experiments. To verify the preservation of the alpha-helix integrity, we performed circular dichroism (CD) spectroscopic measurements. For clarification, the specified variants are marked in bold together with its encoding amino-acid sequence.

Materials and Methods

All synthetic peptides were ordered from TAGCopehagen, and were synthesized by Fmoc based solid phase peptide synthesis, and delivered as >95% pure, as validated by U PLC and LC-MS. All peptides contained an N-terminal Biotin conjugated to the peptide via 6-aminohexanoic acid (Ahx) linkage.

Size exclusion chromatography (SEC): Size exclusion chromatography was performed using an Äkta purifier with a Superdex200 Increase 10/300 column, with 400 μM of indicated peptide. Absorbance profile was measured at 250 nm and plotted against elution volume using Graph Pad Prism.

Circular dichroism (CD): Circular dichroism (CD) spectra was recorded using a Jasco J1500 at 25° C. spectrum was recorded from 190-260 nm in 0.1 nm intervals, using a 1 mm cuvette. Indicated peptides were diluted to 8 μM in 50 mM Sodium Phosphate (NaPi) buffer (pH 8), and spectra were collected.

Results

The following PICK1 binding peptides were studied.

GCN4p1-GS4-HWLKV (SEQ ID NO: 99, Dimeric-HWLKV or
GCN4p1-HWLKV):
biotin-ahx-RMKQLEDKVEELLSKNYHLENEVARLKKLV-GGGGS-
HWLKV,
GCN4p1(NQ)-GS4-HWLKV (SEQ ID NO: 75,
GCN4p1(NQ-HWLKV):
biotin-ahx-RMKQLEDKVEELLSKQYHLENEVARLKKLV-GGGGS-
HWLKV,
GCN4p1(LI)-GS4-HWLKV (SEQ ID NO: 81, GCN4p1(LI)-
HWLKV):
biotin-ahx-RIKQIEDKIEEILSKIYHIENEIARIKKLI-GGGGS-
HWLKV,

The following PSD-95 binding peptides were studied.

GCN4p1-GS4-RRTTPV (SEQ ID NO: 100, Dimeric-RRTTPV
or GCN4p1-RRTTPV):
biotin-ahx-RMKQLEDKVEELLSKNYHLENEVARLKKLV-GGGGS-
RRTTPV,
GCN4p1(LI)-GS4-RRTTPV (SEQ ID NO: 84,
GCN4p1(LI)-RRTTPV):
biotin-ahx-RIKQIEDKIEEILSKIYHIENEIARIKKLI-GGGGS-
RRTTPV,

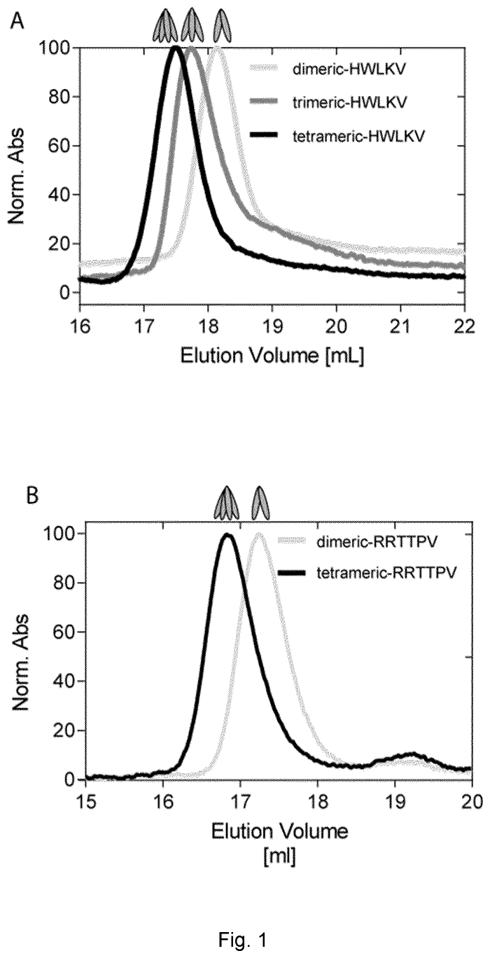

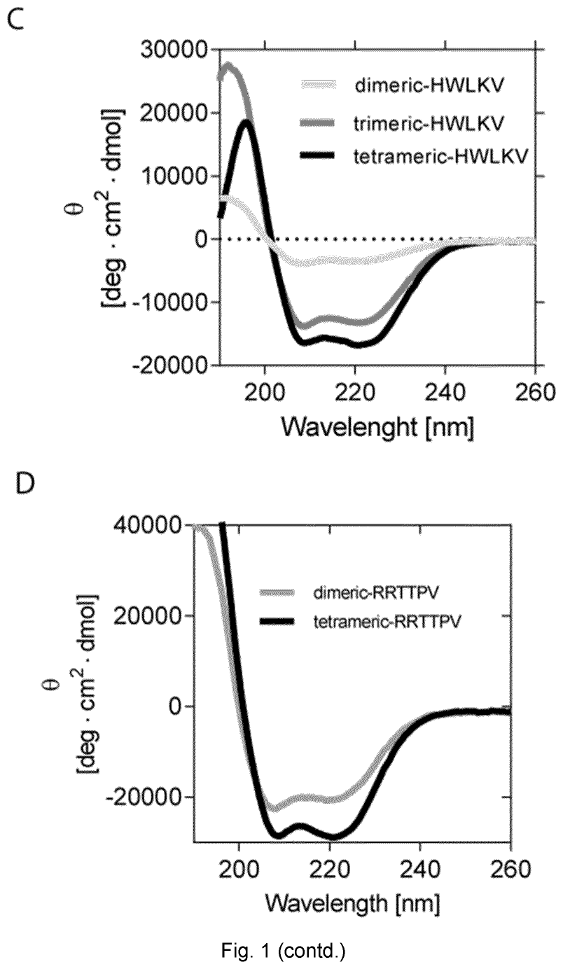

The peptides were analyzed by SEC. GCN4p1 is known to form a dimer, which was confirmed by the SEC analysis (FIGS. 1a and b).

Modification of the GCN4p1 sequence to include glutamine in place of an asparagine at position 16 (N16Q mutation) of the GCN4p1 sequence was found to provide a trimeric construct of peptides. Hence the peptides GCN4p1(NQ)-HWLKV and GCN4p1(NQ)-RRTTPV were found to form trimeric constructs in solution (FIG. 1a).

Modification of the GCN4p1 sequence to include the following mutations (M2I, L5I, V9I, L12I, N16I, L19I, V23I, L26I, and V30I) was found to provide a tetrameric construct of the peptides. Hence the peptides GCN4p1(LI)-HWLKV and GCN4p1(LI)-RRTTPV were found to form tetrameric constructs in solution (FIGS. 1a and b).

The secondary structure of the peptides was analyzed by circular dichroism (CD). All peptides were found to have an alpha-helical structure, confirming that the mutations of the GCN4p1 sequence did not influence the alpha-helical nature. (FIGS. 1c and d).

Conclusion

This example demonstrates the oligomeric nature of the GCN4p1 variants enforced by the specific modifications made to the GCN4p1 amino acid sequence. In summary, the GCN4p1 sequence was successfully modified to provide higher order constructs. The alpha-helical secondary structure of GCN4p1 was found to be conserved for the modified sequences.

### Example 4: Binding of GCN4p1-Variant Containing Peptides to PICK1

In this experiment, we tested whether peptides comprising a PICK1 ligand peptide conjugated to variants of the GCN4p1 backbone, having different oligomeric structure properties, would provide enhanced affinity. Furthermore, the complex size of PICK1 bound to the peptide ligands was studied.

Methods and Materials

Protein expression and purification of PICK1 was performed as presented in Example 1.

Fluorescence Polarization: The competition binding assay was carried out using a fixed concentration of PICK1 (0.19 μM) and fluorescent tracer (10 nM) 5-FAM-NPEG4-(HWLKV)₂ incubated with increasing concentrations of unlabelled peptides using black half-area Corning non-binding surface 96 well plates (Sigma-Aldrich, Ref. no. 3686). The plates were incubated 30-40 min on ice and the fluorescence polarization was measured on an Omega POLARstar plate (BMG LABTECH) reader using excitation filter at 485 nm and long pass emission filter at 520 nm. The data was plotted in GraphPad Prism 8.3 and fitted to a ‘One site—Fit’ Kᵢ curve and the apparent affinities (K) for the unlabelled peptides were determined using correction for depletion.

Size exclusion chromatography: was performed using an Äkta purifier with a Superdex200 Increase 10/300 column, with 500 μL of 40 μM PICK1 in absence or presence of 20 μM dimeric GCN4p1-HWLKV, GCN4p1(NQ)-HWLKV or GCN4p1(LI)-HWLKV. Absorbance profiles were measured at 280 nm and plotted against elution volume using Graph Pad Prism 8.3.

Results

In this series of experiment, we have tested the following peptides targeting PICK1; HWLKV (monomeric pentapeptide, DAT-C5, SEQ ID NO: 54)

GCN4p1-HWLKV (SEQ ID NO: 99):
Bioin-ahx-RMKQLEDKVEELLSKNYHLENEVARLKKLVGGGGSHWLKV
GCN4p1(NQ)-HWLKV (SEQ ID NO: 75):
Bioin-ahx-RMKQLEDKVEELLSKQYHLENEVARLKKLVGGGGSHWLKV
GCN4p1(LI)-HWLKV (SEQ ID NO: 81):
Bioin-ahx-RIKQIEDKIEEILSKIYHIENEIARIKKLIGGGGSHWLKV

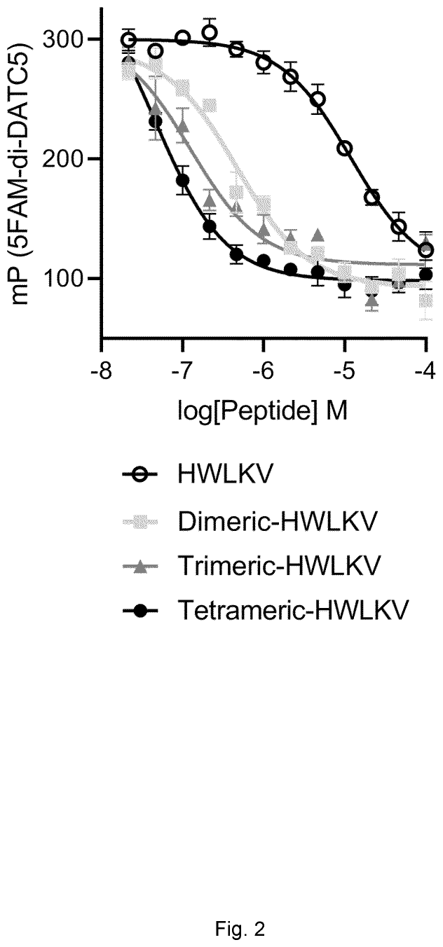

Fluorescent polarization (FP) experiments were performed to determine binding affinities for PICK1. Competition experiment, using 5-FAM-NPEG4-(HWLKV)₂ as fluorescent tracer, demonstrated that GCN4p1(LI)-HWLKV possess the highest affinity for PICK1, approx. a 262 fold shift compared to HWLKV, whereas an approx. 94 fold increase was observed for GCN4p1(NQ)-HWLKV over HWLKV. Both GCN4p1(NQ)-HWLKV and GCN4p1(LI)-HWLKV were found to have higher affinity towards PICK1 as compared to the dimeric GCN4p1-HWLKV, approx. 11 fold and 4 fold, respectively. (FIG. 2). Affinities (Ki) are summarized in the below table, as determined from the ‘One site—Fit’ Kᵢ curve for the unlabelled peptides calculated in GrapPad Prism 8.3.

| Peptide | Affinity (Kᵢ) | |
|---|---|---|
| HWLKV | 2353 | nM |
| Dimeric- GCN4p1HWLKV | 93 | nM |
| GCN4p1(NQ)-HWLKV | 25 | nM |
| GCN4p1(LI)-HWLKV | 9 | nM |

In conclusion, this experiment shows that a higher oligomeric state of the peptide ligands provides enhanced affinity towards PICK1 as compared to monomeric or dimeric peptide ligands.

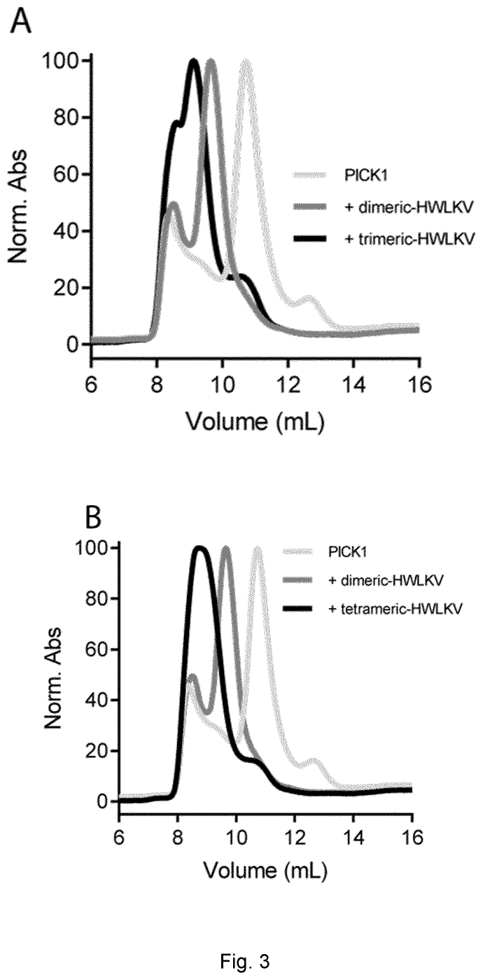

Size exclusion chromatography was performed in order to evaluate the in-solution behavior of PICK1 upon binding to the dimeric GCN4p1-HWLKV, GCN4p1(NQ)-HWLKV and GCN4p1(LI)-HWLKV peptide variants. The shift in elution seen for PICK1 when bound to either GCN4p1(NQ)-HWLKV (FIG. 3A) or GCN4p1(LI)-HWLKV (FIG. 3B) indicates that a larger oligomeric state of the PICK1 protein is induced upon binding to the ligands, GCN4p1(NQ)-HWLKV or GCN4p1(LI)-HWLKV. Furthermore, the data demonstrates that binding of PICK1 to either GCN4p1(NQ)-HWLKV or GCN4p1(LI)-HWLKV results in a larger shift of the elution volume than does binding of PICK1 to dimeric GCN4p1-HWLKV. This indicates that a larger complex of PICK1 protein is formed when PICK1 is bound to GCN4p1(NQ)-HWLKV or GCN4p1(LI)-HWLKV than when it is bound to dimeric GCN4p1-HWLKV. Finally, GCN4p1(LI)-HWLKV was found to shift the elution volume more than GCN4p1(NQ)-HWLKV, which is indicative of a higher oligomeric state of the complex between GCN4p1(LI)-HWLKV and PICK1 as compared to GCN4p1(NQ)-HWLKV bound PICK1.

Conclusion

The present example demonstrates that the higher order constructs of the PICK1 ligand, HWLKV, of the present disclosure result in enhanced affinity of the ligands as compared to the peptide ligand alone, HWLKV or to dimeric GCN4p1-HWLKV. Furthermore, the data shows higher affinity binding to PICK1 when the ligand GCN4p1(LI)-HWLKV is employed as compared to the GCN4p1(NQ)-HWLKV ligand. The present example further demonstrates that the PICK1 inhibitors of the present disclosure is capable of inducing higher order structures of PICK1 upon binding. Inhibition of the protein function is likely to result from such induction of higher order structures of PICK1.

### Example 5: Binding Variants of GCN4p1 to PSD-95

In this experiment, we tested whether peptides comprising a PSD-95 ligand peptide conjugated to variants of the GCN4p1 backbone, having different oligomeric structure properties, would provide enhanced affinity. Furthermore, the complex size of PSD-95 bound to the peptide ligands was studied.

Methods and Materials

Protein expression and purification of PSD-95 was performed as in Example 2.

Fluorescence polarization: Fluorescence polarization was carried out in competition mode at a fixed concentration of protein (150 nM) and tracer (5-FAM-NPEG4-(IETAV)₂, 5 nM, Bach et al. 2012), against an increasing concentration of unlabeled peptide. The plate was incubated 2 hrs on ice in a black half-area Corning Black non-binding surface 96-well plate and the fluorescence polarization was measured directly on a Omega POLARstar plate reader using excitation filter at 488-nm and long pass emission filter at 535-nm. The data was plotted using GraphPad Prism 8.3, and fitted to the One site competition, to extract Ki values.

SDS-PAGE sedimentation assay: Proteins were mixed in the desired concentration in PBS-TCEP and equilibrated for 10 min before centrifugation at 20 000 g for 15 min at 25° C. using a temperature controlled table top centrifuge. Following centrifugation the supernatant was collected and the pellet was re-suspended in an equal amount of PBS-TCEP, usually 50 μL. To ensure proper resuspension of LLPS, the samples were vortexed before addition of SDS buffer followed by boiling at 95° C. for 5 min. Supernatant and pellet fractions were run on any kD™ Mini-PROTEAN® TGX™ Precast Protein Gels (10 or 15 wells, BioRad 4569036 or 4569033). Gels were imaged using a Li-COR Odyssey gel scanner and band intensities were analyzed using ImageJ.

Confocal microscopy on liquid-liquid phase separation droplets: Confocal microscopy was performed using a Zeiss LSM780 using a 63×NA 1.4 plan apochromat oil objective using Argon 488 nm 25 mW, 543 nm HeNe 1.2 mW and 633 nm HeNe 5 mW lasers using a detection wavelength of 490-538 nm for the 488 channel, 556-627 nm for the 543 channel, 636-758 for the 633 channel. Images were acquired using averaging of 4 line scans and 12-bit. The liquid-liquid phase separation droplets were prepared in the desired concentration in PBS-TCEP and added to an untreated lab tec (155411PK) and imaged after being allowed to settle for 15 min at 25° C. For samples containing fluorescent protein or peptide the content of fluorescent protein or peptide was kept at 10% of indicated total protein or peptide concentration.

Results

In this series of experiment, we have tested the following two PSD-95 binding peptides;

(SEQ ID NO: 65)
SNTANRRTTPV
Monomeric-RRTTPV
(GCN4p1(7P14P)-GS4-RRTTPV, SEQ ID NO: 101):
Biotin-ahx-RMKQLEPKVEELLPKNYHLENEVARLKKLVGGGGSRRTT
PV
Dimeric-RRTTPV
(GCN4p1-GS4-RRTTPV, SEQ ID NO: 100):
Biotin-ahx-RMKQLEDKVEELLSKNYHLENEVARLKKLVGGGGSRRTT
PV
GCN4p1(LI)-GS4-RRTTPV, (SEQ ID NO: 84):
Biotin-ahx-RIKQIEDKIEEILSKIYHIENEIARIKKLIGGGGSRRTT
PV
Monomeric-IETDV
(GCN4p1(7P14P)-GS4-IETDV, SEQ ID NO: 103):
Biotin-ahx-RMKQLEPKVEELLPKNYHLENEVARLKKLVGGGGSIET
DV
Dimeric-IETDV
(GCN4p1-GS4-IETDV, SEQ ID NO: 102):
Biotin-ahx-RMKQLEDKVEELLSKNYHLENEVARLKKLVGGGGSIET
DV
GCN4p1(LI)-GS4-IETDV, (SEQ ID NO: 83):
Biotin-ahx-RIKQIEDKIEEILSKIYHIENEIARIKKLIGGGGSIET
DV

Peptides quaternary structure: Dimeric GCN4p1-IETDV and Dimeric GCN4p1-RRTTPV both comprise the GCN4p1 variant which was demonstrated to provide a dimeric quaternary structure of the peptide in solution for the peptides GCN4p1-HWLKV and GCN4p1-RRTTPV (Example 3). Furthermore, an alpha-helical secondary structure was confirmed for both peptides. This demonstrates that the C-terminal peptide ligand (HWLKV or RRTTPV) has no effect on the alpha-helical nature of the peptide or on the quaternary structure of the peptide in solution. Hence it is reasonable to suggest that GCN4p1-IETDV (dimeric GCN4p1-IETDV) has the same structural properties, i.e. being an alpha helix and a dimer in solution. The same argumentation holds for GCN4p1(LI)-IETDV being a higher order oligomer, trimer or tetramer, as demonstrated in examples 3 or 7.

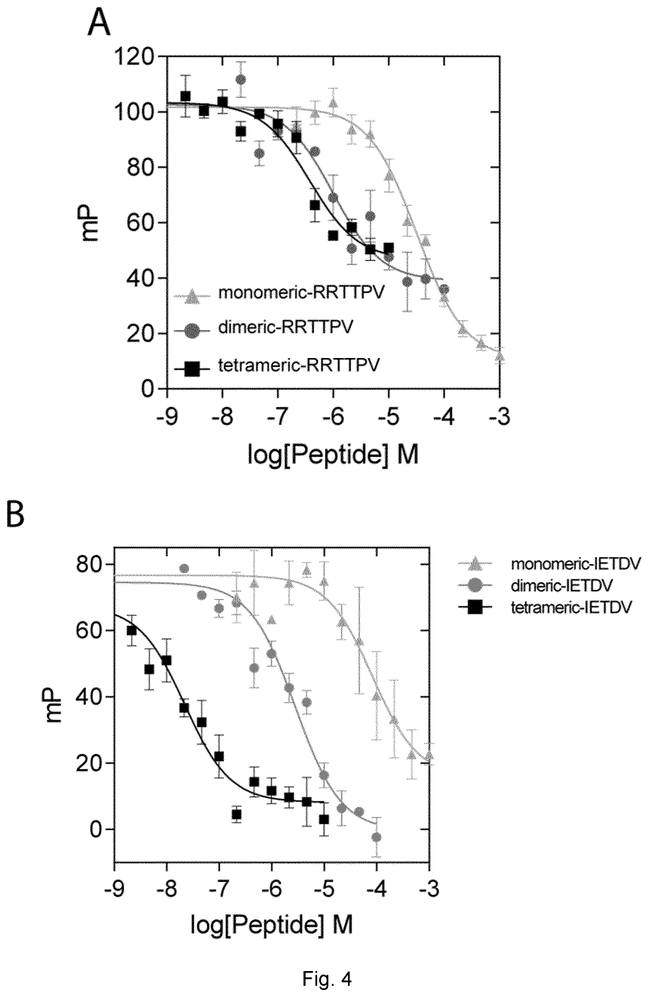

Fluorescent polarization experiments were performed to determine binding affinity for PSD-95. Competition experiment, using 5-FAM-NPEG4-(IETAV)₂ as fluorescent tracer, demonstrated the highest affinity for GCN4p1(LI)-IETDV, approx. a 3811-fold shift compared to monomeric GCN4p1(7P14P)-IETDV (FIG. 4A), whereas an approx. 84 fold increase was observed for GCN4p1(LI)-RRTTPV over monomeric GCN4p1(7P14P)-RRTTPV (FIG. 4B). Both GCN4p1(LI)-IETDV and GCN4p1(LI)-RRTTPV were found to have higher affinity towards PSD-95 as compared to dimeric GCN4p1-IETDV or dimeric GCN4p1-RRTTPV, approx. 125 fold and approx. 2.6 fold, respectively. Affinities (Ki) are summarized in the below table, as determined from the ‘One site—Fit’ Kᵢ curve for the unlabelled peptides calculated in GrapPad Prism 8.3.

| Peptide | Affinity (Kᵢ) | |
|---|---|---|
| Monomeric GCN4p1(7P14P)-RRTTPV | 7998 | nM |
| Dimeric GCN4p1-RRTTPV | 246 | nM |
| GCN4p1(LI)-RRTTPV | 95 | nM |
| Monomeric GCN4p1(7P14P)-IETDV | 20810 | nM |
| Dimeric GCN4p1-IETDV | 702 | nM |
| GCN4p1(LI)-IETDV | 5.5 | nM |

In conclusion, this experiment demonstrates that a higher oligomeric state of PSD-95 peptide ligands provides enhanced affinity towards PSD-95.

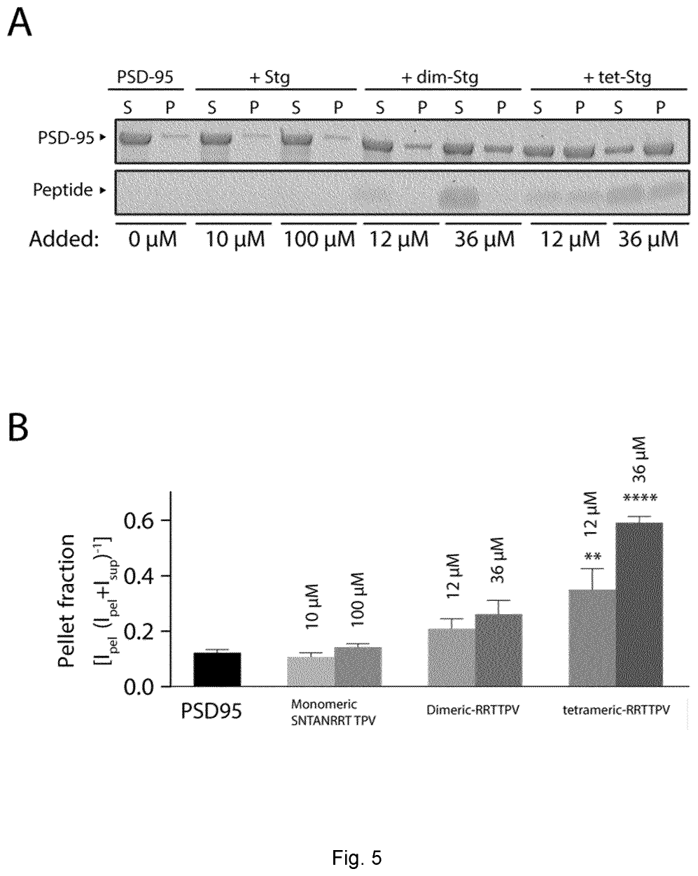

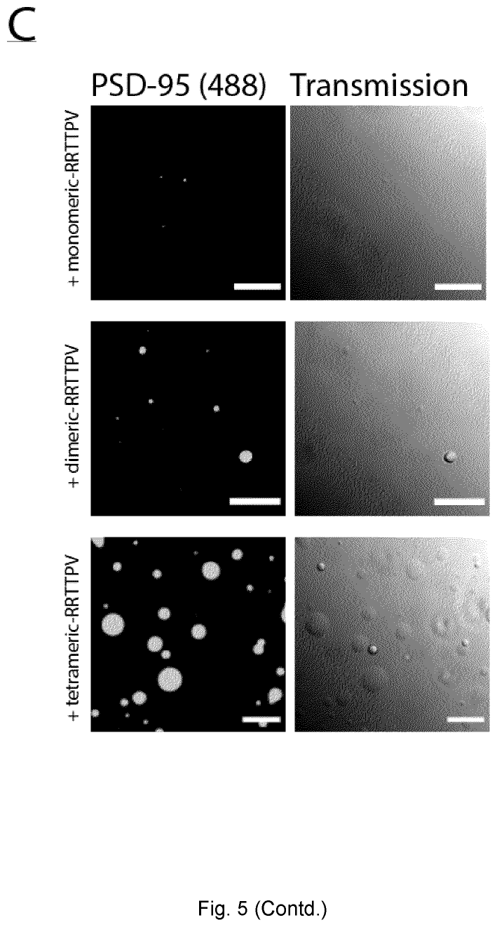

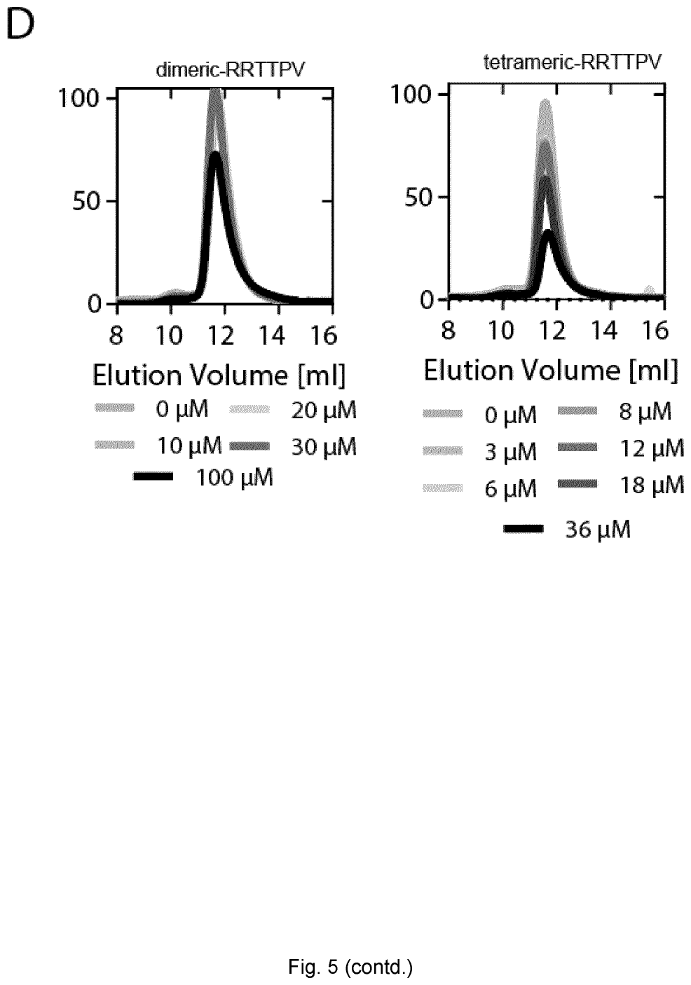

To evaluate whether the GCN4p1(LI)-RRTTPV was able to induce a higher oligomeric conformation of PSD-95, PSD-95 bound to GCN4p1(LI)-RRTTPV was analysed by size exclusion chromatography (SEC) and compared to dimeric GCN4p1-RRTTPV. To our surprise, we found that upon an increase in dimeric GCN4p1-RRTTPV and GCN4p1(LI)-RRTTPV concentration relative to PSD-95 concentration we observed a reduction in the total amount of PSD-95 eluting from the column (FIG. 5d), with no peak at lower elution volume observed. This reduction of total amount eluting from the column was more pronounced for PSD-95 bound to GCN4p1(LI)-RRTTPV as compared to PSD-95 bound to dimeric GCN4p1-RRTTPV. We therefore investigated this phenomenon further using a SDS page protein sedimentation assay.

SDS-PAGE sedimentation was performed in order to evaluate the in solution behavior of PSD-95 in complex with SNTANRRTTPV peptide, dimeric GCN4p1-RRTTPV or GCN4p1(LI)-RRTTPV.

The SDS-PAGE sedimentation assay demonstrated that GCN4p1(LI)-RRTTPV, induced a cloudy phase which could be pelleted (FIGS. 5a and b). The supernatant and pellet were analyzed by SDS-page revealing that in the absence of peptide ligand, the majority of PSD-95 is found in the supernatant. The same applies to PSD-95 bound to SNTANRRTTPV peptide. Incubation of PSD-95 with dimeric GCN4p1-RRTTPV resulted in some PSD-95 being present in the pellet, with the majority being present in the supernatant. Incubation with GCN4p1(LI)-RRTTPV provided a shift with the majority of the PSD-95 being present in the pellet, indicating that GCN4p1(LI)-RRTTPV, forms higher order oligomeric structures, with PSD-95 leading to liquid-liquid phase separation.

To further evaluate if GCN4p1(LI)-RRTTPV induced a liquid-liquid phase separation (LLPS) transition, we performed fluorescence confocal microscopy of Alexa488-labeled PSD-95 bound to unlabelled peptides (GCN4p1(LI)-RRTTPV). Indeed, we found that mixing GCN4p1(LI)-RRTTPV (at 36 μM) with PSD-95 (3 μM) induced LLPS droplets, while this was not the case for monomeric GCN4p1(7P14P)-RRTTPV, and only to a lesser extent for the dimeric GCN4p1-RRTTPV peptide (FIG. 5c). The formation of LLPS droplets suggests, that higher order structure of peptide ligands, such as our GCN4p1(LI)-RRTTPV containing four copies of a PSD-95 peptide ligand, is able to induce LLPS when mixed with PSD-95.

Conclusion

The present example demonstrates that the higher order constructs of the PSD-95 ligands, RRTTPV and IETDV, of the present disclosure result in enhanced affinity of the ligands as compared to the peptide ligand alone or to dimeric ligand constructs. The present example further demonstrates that the constructs of the PSD-95 ligands comprising GCN4p1(LI) as the first polypeptide part of the present disclosure is capable of inducing higher order structures of PSD-95 upon binding, resulting in LLPS. Inhibition of the protein function is likely to result from such induction of higher order structures of PSD-95.

### Example 6: Optimizing the Sequence of the PICK1 Binding Peptide Ligand to Identify High Affinity Binders

To test the stringency of the PICK1 PDZ binding motif in the DAT-C5 (HWLKV) sequence (i.e. position X₁-X₅) and to indicate putatively peptides with better affinity, we performed an initial study using fluorescence polarization binding to purified PICK1 of 95 different penta-peptides with each residue in the HWLKV sequence substituted to either of the 19 other natural amino acids.

Further, we took the data obtained in the above experiment, and utilized it for guidance to design 52 different penta-, tetra and tri-peptides, derived from combinatorial substitution of amino acids. To verify putative peptides with better affinity, binding affinities to purified PICK1 were studied by fluorescence polarization binding assays.

Materials and Methods:

Peptides were ordered from TAG Copenhagen Aps, as >95% purity, validated by UPLC and LC-MS.

Fluorescence polarization: Fluorescence polarization was carried out in competition mode at a fixed concentration of protein and tracer (5FAM-HWLKV, 20 nM), against an increasing concentration of indicated unlabeled peptide. The plate was incubated 20 min on ice in a black half-area Corning Black non-binding surface 96-well plate and the fluorescence polarization was measured directly on a Omega POLARstar plate reader using excitation filter at 488-nm and long pass emission filter at 535-nm. The data was plotted using GraphPad Prism 6.0, and fitted to the One-site competition, to extract Kᵢ values, which were all correlated to the HWLKV affinity, which was finally plotted.

Results

Single Substitution Experiment:

Substitution of X₁ and X₃ was mostly disruptive to binding (indicated by lighter shades) except for substitution of X₃ to V and I, which increased affinity (FIG. 5). On position X₂, substitutions to R, C, I and L all increased affinity and most substitutions were tolerated. Likewise, substitutions of X₄ and X₅ were in general well tolerated with notable exceptions of positively charged residues in X₄, which decreased affinity. Substitution to Y, E, S, Q, C, A and G in position X₄, increased affinity. Most substitutions (including Y, F, T, S, Q, N, C, V, M, I, G, A) increased affinity in position X₅ albeit several substitutions compromised solubility.

52 Combinatorial Peptides:

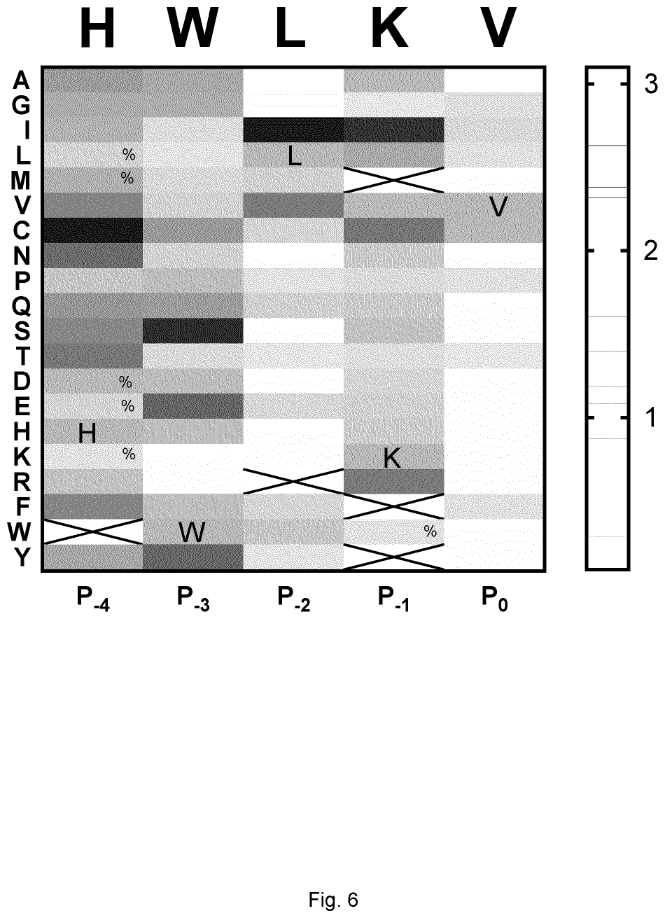

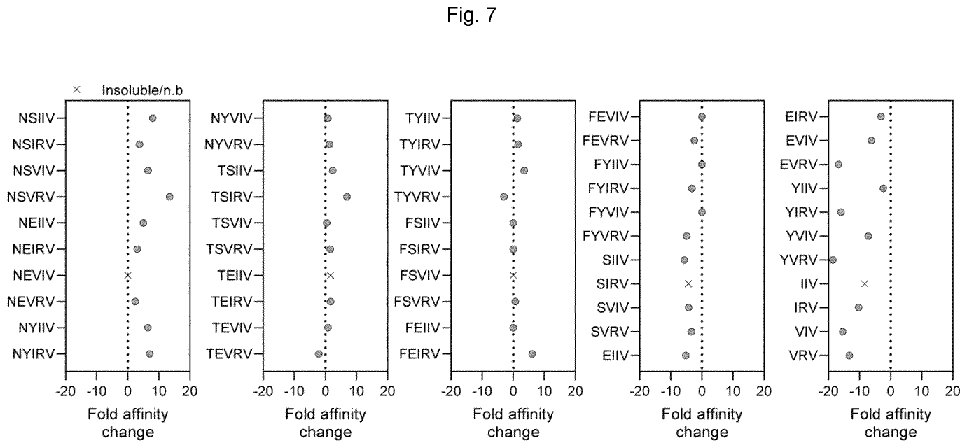

Based on double substitutions in class II binding motifs we found that many combinations were well tolerated, and in general N at position X₁, S/E at position X₂, R at position X₄ had a better or non-perturbed affinity, while F at position X₁ was, in general, not as well tolerated (FIG. 6).

Conclusion

This example demonstrates that optimization of the HWLKV sequence by amino acid substitutions provide peptide ligands showing equivalent and even higher affinity towards PICK1.

### Example 7: Oligomer Formation of Oligomerization Domains

Different, non GCN4p1, oligomerization domains as well as GCN4p1 variants were studied to determine the oligomerization state.

Materials and Methods:

The following non GCN4p1 oligomerization domains were studied:

CC-Hex2; GEIAKSLKEIAKSLKEIAWSLKEIAKSLK
(A. R. Thomson et al, 2014 Science,
PDB deposit 4PN9, SEQ ID NO: 70)
CC-tet; GELAAIKQELAAIKKELAAIKWELAAIKQ.
Zaccai et al., 2011 Nat. Chem. Biol.,
PDB deposit 3R4A, SEQ ID NO: 69)

The following peptides were studied in this example:

GCN4p1 variants (dimeric control peptides):
GCN4p1-GS4-HWLKV (SEQ ID NO: 99);
biotin-ahx-RMKQLEDKVEELLSKNYHLENEVARLKKLV-GGGGS-
HWLKV
GCN4p1-GS4-IETDV (SEQ ID NO: 102);
biotin-ahx-RMKQLEDKVEELLSKNYHLENEVARLKKLV-GGGGS-
IETDV
GCN4p1(LI) variants:
GCN4p1(LI)-GS4-IETDV (SEQ ID NO: 83);
biotin-ahx-RIKQIEDKIEEILSKIYHIENEIARIKKLI-GGGGS-
IETDV
GCN4p1(LI)-GS4-HWLKV (SEQ ID NO: 81);
biotin-ahx-RIKQIEDKIEEILSKIYHIENEIARIKKLI-GGGGS-
HWLKV
GCN4p1(LI)-GS4-NSVRV (SEQ ID NO: 144);
biotin-ahx-RIKQIEDKIEEILSKIYHIENEIARIKKLI-GGGGS-
NSVRV
GCN4p1(LI)-GS4-YKQTSV (SEQ ID NO: 85);
biotin-ahx-RIKQIEDKIEEILSKIYHIENEIARIKKLI-GGGGS-
YKQTSV
GCN4p1(NQ) variants:
GCN4p1(NQ)-GS4-HWLKV (SEQ ID NO: 75);
biotin-ahx-RMKQLEDKVEELLSKQYHLENEVARLKKLV-GGGGS-
HWLKV
CC-Tet variants:
CC-tet-GS4-HWLKV (SEQ ID NO: 87):
biotin-ahx-GELAAIKQELAAIKKELAAIKWELAAIKQ-GGGGS-
HWLKV
GCN4p1(ILI) variants:
GCN4p1(ILI)-GS4-HWLKV (SEQ ID NO: 148);
Biotin-ahx-RMKQIEDKLEEILSKLYHIENELARIKKLL-GGGGS-
HWLKV
GCN4p1(ILI)-GS4-NSVRV (SEQ ID NO: 149);
Biotin-ahx-RMKQIEDKLEEILSKLYHIENELARIKKLL-GGGGS-
NSVRV
GCN4p1(ILI)-GS4-IETDV (SEQ ID NO: 150);
Biotin-ahx-RMKQIEDKLEEILSKLYHIENELARIKKLL-GGGGS-
IETDV
GCN4p1(ILI)-GS4-RRTTPV (SEQ ID NO: 151);
Biotin-ahx-RMKQIEDKLEEILSKLYHIENELARIKKLL-GGGGS-
RRTTPV
CC-Hex2 variants:
CC-Hex2-GS4-HWLKV (SEQ ID NO: 93):
biotin-ahx-GEIAKSLKEIAKSLKEIAWSLKEIAKSLK-GGGGS-
HWLKV
CC-Hex2-GS4-RRTTPV (SEQ ID NO: 96):
biotin-ahx-GEIAKSLKEIAKSLKEIAWSLKEIAKSLK-GGGGS-
RRTTPV

Size exclusion chromatography Multi angle light scattering (SEC-MALS): was done using an Agilent HPLC equipped with a Wyatt MALS setup, where 50 μL of 1000 μM, of indicated peptide, was loaded onto a Superdex200 Increase 10/300 column. Resulting data was analyzed and molecular weight was calculated using the ASTRA® software package, data was plotted using GraphPad Prism 8.3.

Flow induced dispersion analysis (FIDA): FIDA was carried out using intrinsic fluorescence at 256 μM of indicated peptide, using the standard protocol recommended by the manufacturer, in short, a peptide and buffer sample was loaded to the FIDA1 instrument, and peptide sample was injected into the capillary followed by a buffer injection. The diffusion of the peptide could then be observed using intrinsic fluorescence, and the hydrodynamic radius was calculated using the FIDA software 2.0 using a single guassian distribution fit, at 75% and curve smoothing. Resulting hydrodynamic radius was plotted using GraphPad Prism 8.3.

Circular dichroism (CD): Circular dichroism (CD) spectra were recorded using a Jasco J1500 at 25° C., spectrum was recorded from 190-260 nm in 0.1 nm intervals, using a 1 mm cuvette. Indicated peptides were diluted to 8 μM in 50 mM Sodium Phosphate (NaPi) buffer (pH 8), and spectra was collected.

Peptides: All peptides were bought from TAGCopenhagen, and were synthesized by standard SPPS chemistry. In all cases the peptide purity was >95%, which was validated by LC-MS and UPLC.

Results:

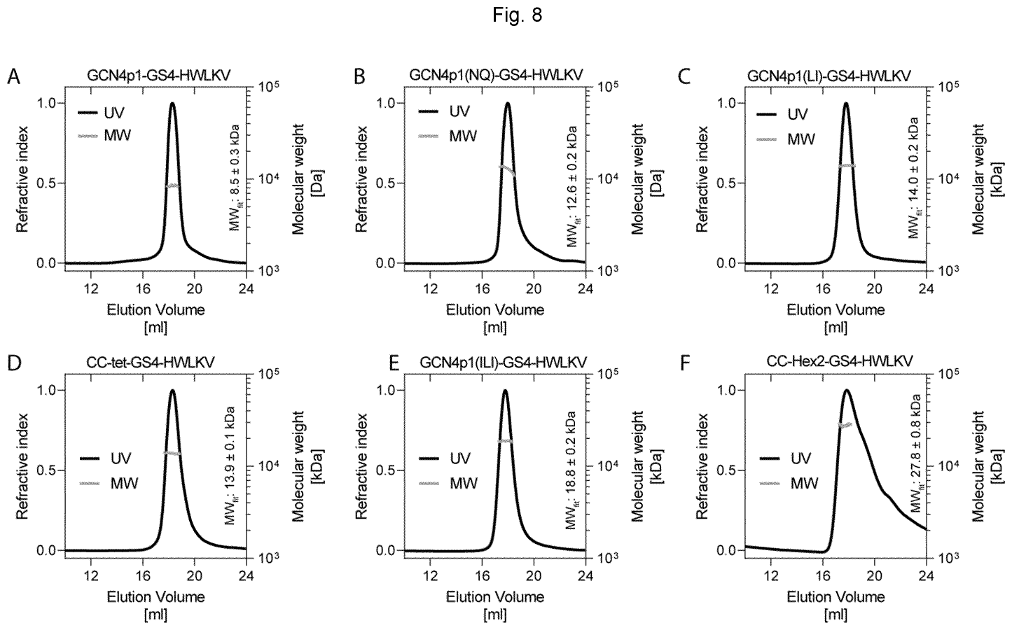

The data of the SEC-MALS experiment suggests that GCN4p1-GS4-HWLKV is in a dimeric configuration, GCN4p1(NQ)-GS4-HWLKV, GCN4p1(LI)-GS4-HWLKV and CC-tet-GS4-HWLKV are in a trimeric configuration, GCN4p1(ILI)-GS4-HWLKV is in tetrameric configuration, and CC-hex-GS4-HWLKV is in a hexameric configuration (FIG. 8).

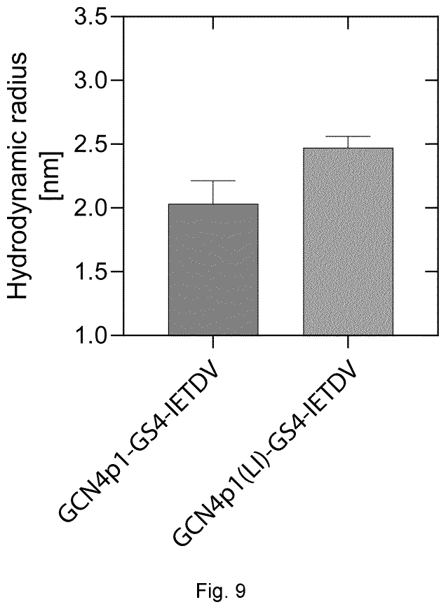

The data of the FIDA experiment demonstrate a larger hydrodynamic radius of GCN4p1(LI)-GS4-IETDV than of GCN4p1-GS4-IETDV, suggesting a larger oligomeric state of GCN4p1(LI)-GS4-IETDV than the dimeric state of GCN4p1-GS4-IETDV (FIG. 9).

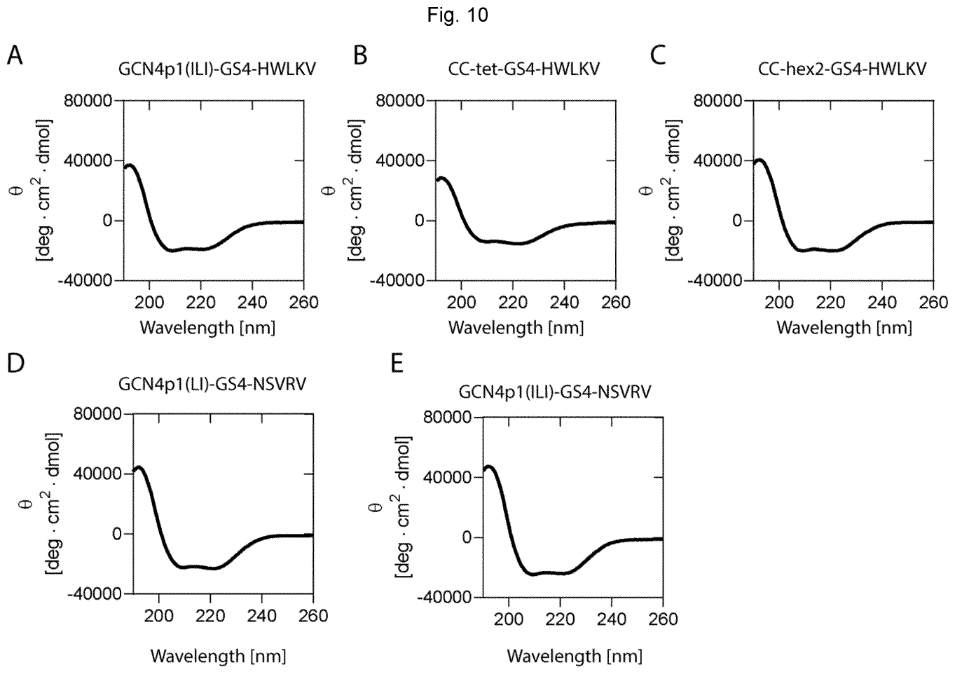

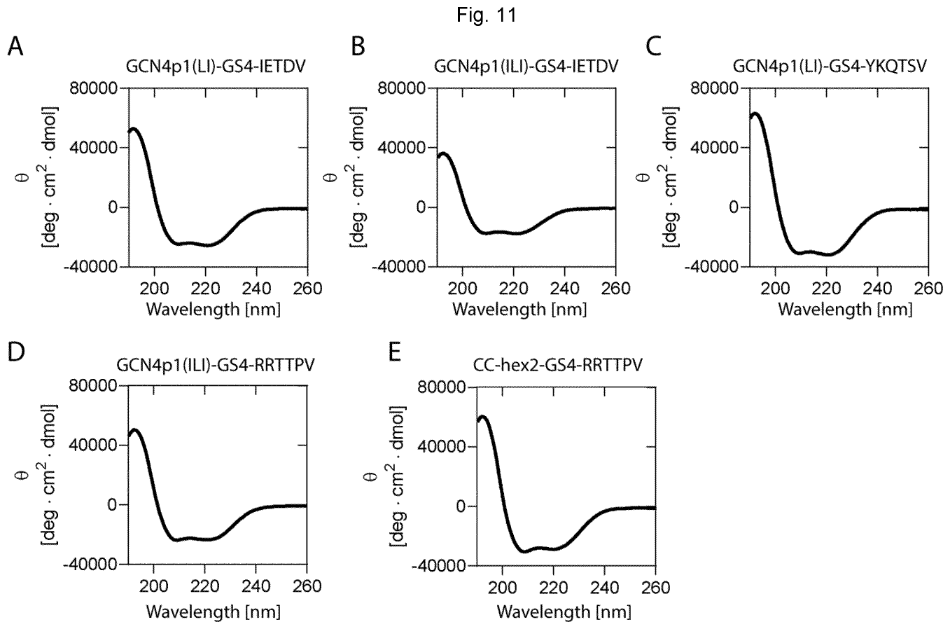

Circular dichroism spectra validated a high degree of helical structure of all the peptides: GCN4p1(ILI)-GS4-HWLKV, CC-tet-GS4-HWLKV, CC-Hex2-GS4-HWLKV, GCN4p1(LI)-GS4-NSVRV, and GCN4p1(ILI)-GS4-NSVRV (FIG. 10) as well as of the peptides: GCN4p1(LI)-GS4-IETDV, GCN4p1(IL1)-GS4-IETDV, GCN4p1(LI)-GS4-YKQTSV, GCN4p1(ILI)-GS4-RRTTPV, and CC-Hex2-GS4-RRTTPV (FIG. 11).

Conclusion:

This example demonstrate methods for determining the oligomeric state of peptides comprising an oligomization domain linked to a PBM. The example demonstrates that the tested peptides range in oligomerization state between dimers (control peptides), trimers, tetramers, and hexamers, depending on the sequence of the oligomerization domain. The example further demonstrates that the peptides have an overall alpha-helical structure.

### Example 8: Higher Order Oligomers with Variation in the Oligomerization Domain

Different oligomerization domains, non GCN4p1 sequence as well as GCN4p1 variants were tested in combination with different Class I and II PDZ binding motifs.

Materials and Methods:

The following peptides were studied in this example:

GCN4p1 variants (dimeric control peptides);
GCN4p1-GS4-HWLKV (SEQ ID NO: 99);
biotin-ahx-RMKQLEDKVEELLSKNYHLENEVARLKKLV-GGGGS-
HWLKV
GCN4p1-GS4-NSVRV (SEQ ID NO: 145);
biotin-ahx-RMKQLEDKVEELLSKNYHLENEVARLKKLV-GGGGS-
NSVRV
GCN4p1-GS4-IETDV (SEQ ID NO: 102);
biotin-ahx-RMKQLEDKVEELLSKNYHLENEVARLKKLV-GGGGS-
IETDV
GCN4p1-GS4-RRTTPV (SEQ ID NO: 100);
biotin-ahx-RMKQLEDKVEELLSKNYHLENEVARLKKLV-GGGGS-
RRTTPV
GCN4p1(LI) variants;
GCN4p1(LI)-GS4-IETDV (SEQ ID NO: 83);
biotin-ahx-RIKQIEDKIEEILSKIYHIENEIARIKKLI-GGGGS-
IETDV
GCN4p1(LI)-GS4-RRTTPV (SEQ ID NO: 84);
biotin-ahx-RIKQIEDKIEEILSKIYHIENEIARIKKLI-GGGGS-
RRTTPV
GCN4p1(LI)-GS4-HWLKV (SEQ ID NO: 81);
biotin-ahx-RIKQIEDKIEEILSKIYHIENEIARIKKLI-GGGGS-
HWLKV
GCN4p1(LI)-GS4-NSVRV (SEQ ID NO: 144);
biotin-ahx-RIKQIEDKIEEILSKIYHIENEIARIKKLI-GGGGS-
NSVRV
GCN4p1(ILI) variants;
GCN4p1(ILI)-GS4-IETDV (SEQ ID NO: 150);
biotin-ahx-RMKQIEDKLEEILSKLYHIENELARIKKLL-GGGGS-
IETDV
GCN4p1(ILI)-GS4-RRTTPV (SEQ ID NO: 151);
biotin-ahx-RMKQIEDKLEEILSKLYHIENELARIKKLL-GGGGS-
RRTTPV
CC-Tet variants;
CC-tet-GS4-HWLKV (SEQ ID NO: 87):
biotin-ahx- GELAAIKQELAAIKKELAAIKWELAAIKQ-GGGGS-
HWLKV
CC-Hex2 variants;
CC-Hex2-GS4-HWLKV (SEQ ID NO: 93):
biotin-ahx-GEIAKSLKEIAKSLKEIAWSLKEIAKSLK-GGGGS-
HWLKV
CC-Hex2-GS4-NSVRV(SEQ ID NO: 146):
biotin-ahx-GEIAKSLKEIAKSLKEIAWSLKEIAKSLK-GGGGS-
NSVRV
CC-Hex2-GS4-IETDV (SEQ ID NO: 95):
biotin-ahx-GEIAKSLKEIAKSLKEIAWSLKEIAKSLK-GGGGS-
IETDV
CC-Hex2-GS4-RRTTPV (SEQ ID NO: 96):
biotin-ahx-GEIAKSLKEIAKSLKEIAWSLKEIAKSLK-GGGGS-
RRTTPV

Fluorescence Polarization for PICK1: The competition binding assay was carried out using a fixed concentration of PICK1 (0.19 μM) and fluorescent tracer (10 nM) 5-FAM-(HWLKV)₂ incubated with increasing concentrations of unlabelled peptides using black half-area Corning non-binding surface 96 well plates (Sigma-Aldrich, Ref. no. 3686). The plates were incubated 30-40 min on ice and the fluorescence polarization was measured on an Omega POLARstar plate (BMG LABTECH) reader using excitation filter at 485 nm and long pass emission filter at 520 nm. The data was plotted using GraphPad Prism 8.3, and fitted to the ‘One site—Fit’ Kᵢ competition curve, to extract apparent KI values.

Fluorescence polarization for PSD-95 (FL and PDZ12): Fluorescence polarization was carried out in competition mode at a fixed concentration of protein (150 nM) and tracer (5FAM-(IETAV)₂, 5 nM), against an increasing concentration of unlabeled peptide. The plate was incubated 1-2 hrs on ice in a black half-area Corning Black non-binding surface 96-well plate and the fluorescence polarization was measured directly on a Omega POLARstar plate reader using excitation filter at 488-nm and long pass emission filter at 535-nm. The data was plotted using GraphPad Prism 8.3, and fitted to the ‘One site—Fit’ Kᵢ competition curve, to extract apparent KI values.

Size exclusion chromatography: was done using a Äkta purifier with a Superdex200 Increase 10/300 column, where, 500 μL of 30 μM PICK1 or 200 μL 10 uM of FL-PSD-95 in absence or presence of peptides was loaded. Absorbance profile was measured at 280 nm and plotted against elution volume using Graph Pad Prism 8.3.

Confocal microscopy on liquid-liquid phase separation droplets: Confocal microscopy was done using a Zeiss LSM780 equipped with a 63×NA 1.4 plan apochromat oil objective using Argon 488 nm 25 mW, 543 nm HeNe 1.2 mW and 633 nm HeNe 5 mW lasers using a detection wavelength of 490-538 nm for the 488 channel, 556-627 nm for the 543 channel, 636-758 for the 633 channel. Images were acquired using averaging of 4 line scans and 12-bit. The liquid-liquid phase separation droplets were prepared in the desired concentration in Phosphate buffered Saline supplemented with 1 mM TCEP (PBS-TCEP) and added to an untreated lab tec (155411PK) and imaged after being allowed to settle for 5 min at 25° C. For samples containing fluorescent protein or peptide the content of fluorescent protein or peptide was kept at 1% of indicated total protein or peptide concentration.

Results

Protein Oligomer Formation

Pick1

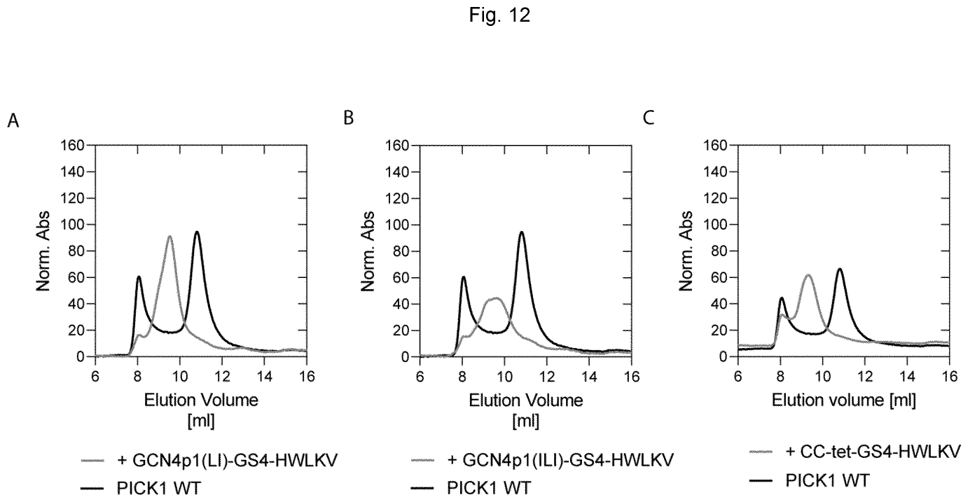

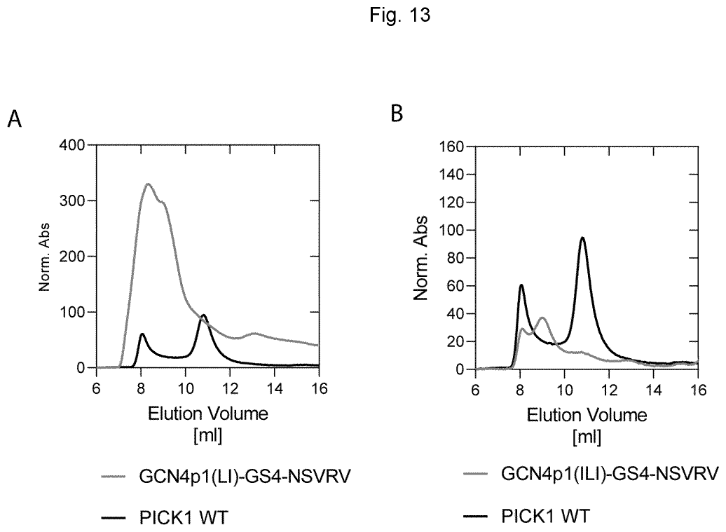

When incubated with PICK1, GCN4p1(LI)-GS4-HWLKV, GCN4p1(ILI)-GS4-HWLKV, and CC-tet-GS4-HWLKV displayed ability to form higher order oligomers of PICK1 (FIG. 12). The same was observed for GCN4p1(LI)-GS4-NSVRV and GCN4p1(ILI)-GS4-NSVRV (FIG. 13).

PSD-95

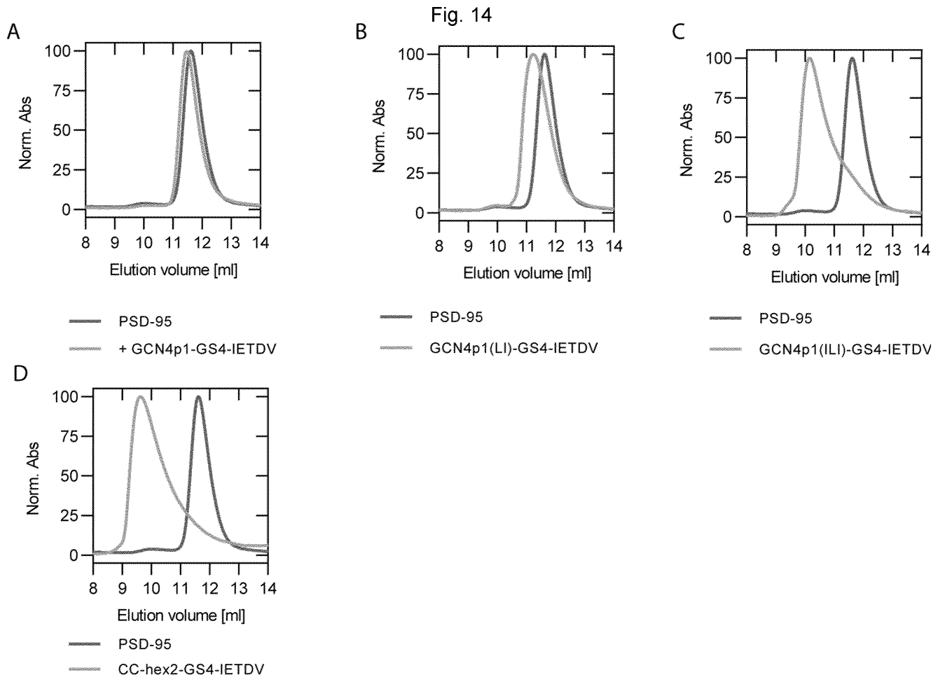

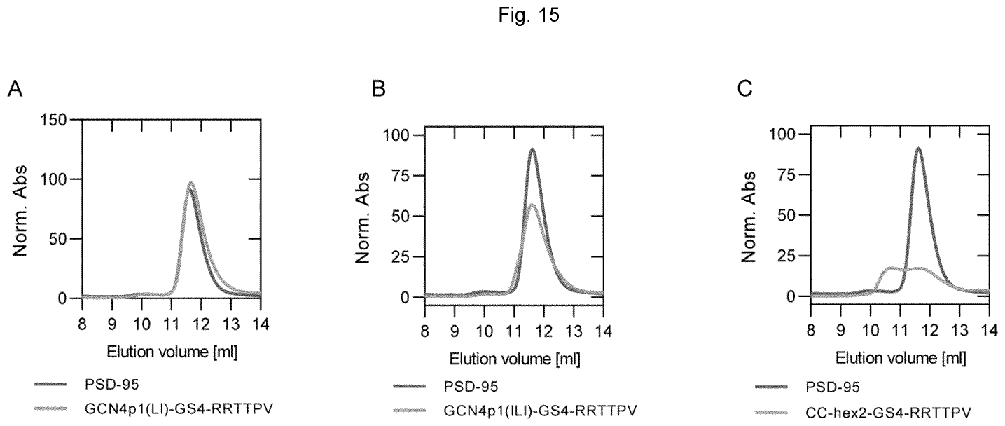

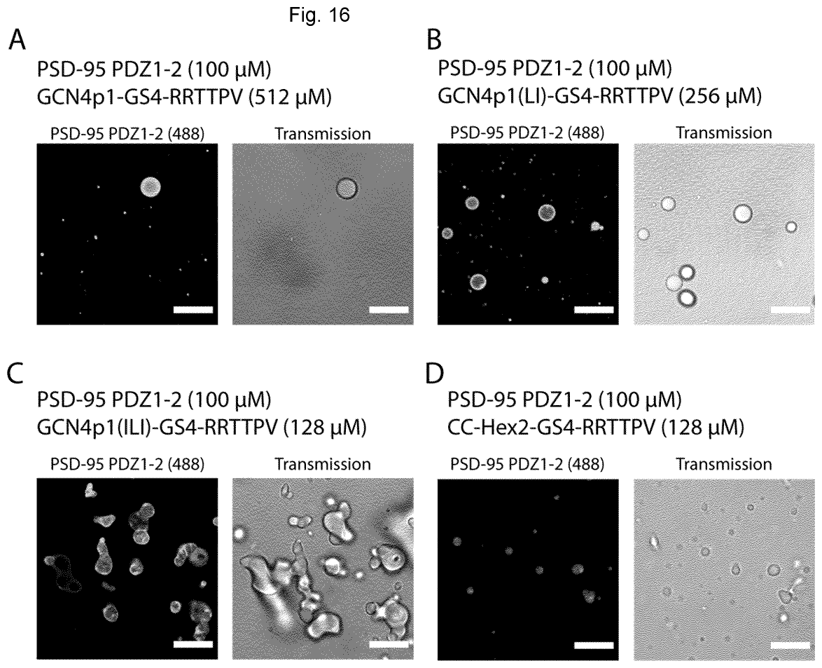

When incubated with PSD-95, GCN4p1(LI)-GS4-IETDV, GCN4p1(IL1)-GS4-IETDV, and CC-Hex2-GS4-IETDV displayed ability to form higher order oligomers of PSD-95, whereas this ability was not observed for dimeric GCN4p1-GS4-IETDV (FIG. 14). Formation of higher order oligomers of PSD-95 was also observed for GCN4p1(ILI)-GS4-RRTTPV and CC-Hex2-GS4-RRTTPV, whereas dimeric GCN4p1-GS4-RRTTPV was not able to induce higher order oligomers of PSD-95 (FIG. 15). For CC-Hex2-GS4-RRTTPV the oligomerization of PSD-95 was evident as a reduced max peak height (FIG. 15C) due to formation of liquid-liquid phase separation (FIG. 16). In the case of LLPS formation, GCN4p1(LI)-GS4-RRTTPV, GCN4p1(ILI)-GS4-RRTTPV, and CC-Hex2-GS4-RRTTPV showed lowered thresholds for LLPS formation of PSD-95 PDZ1-2 than was the case for dimeric GCN4p1-GS4-RRTTPV (FIG. 16).

Binding Affinity

PICK1

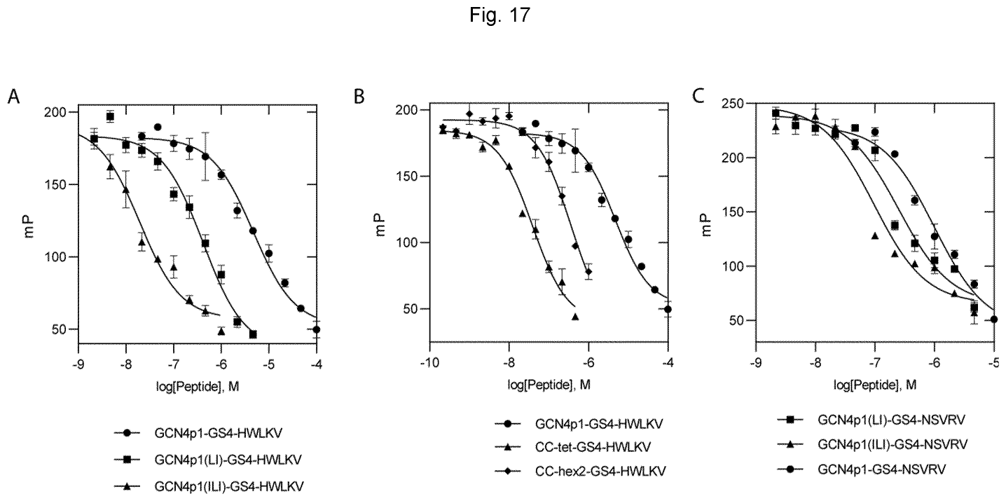

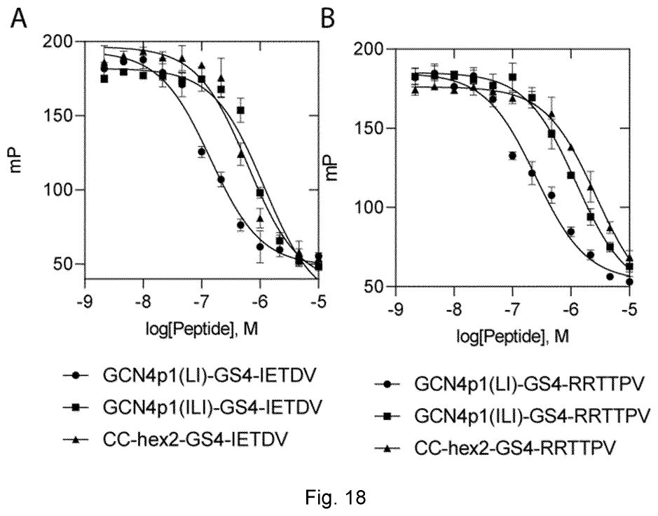

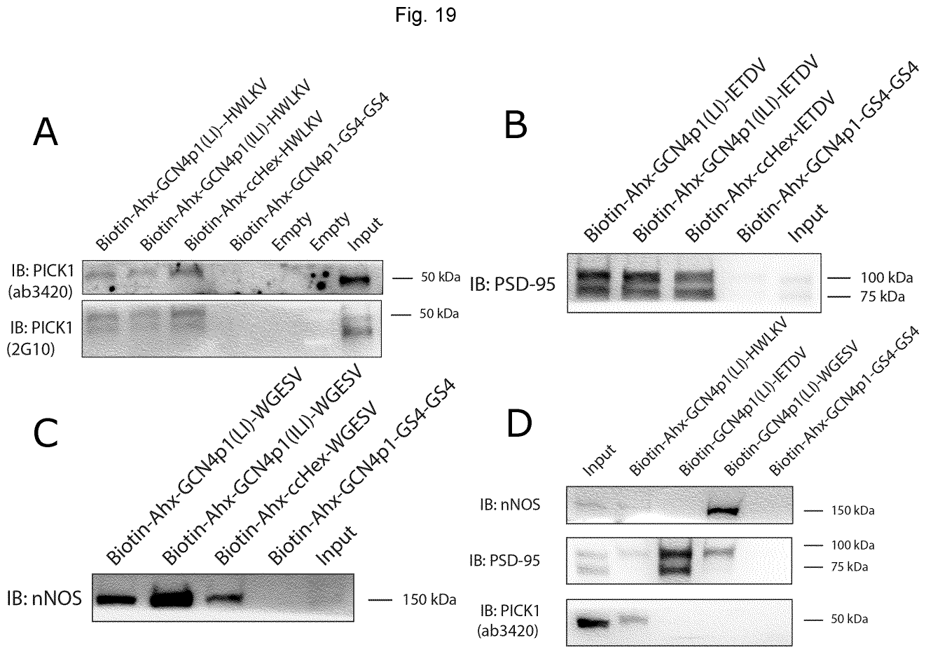

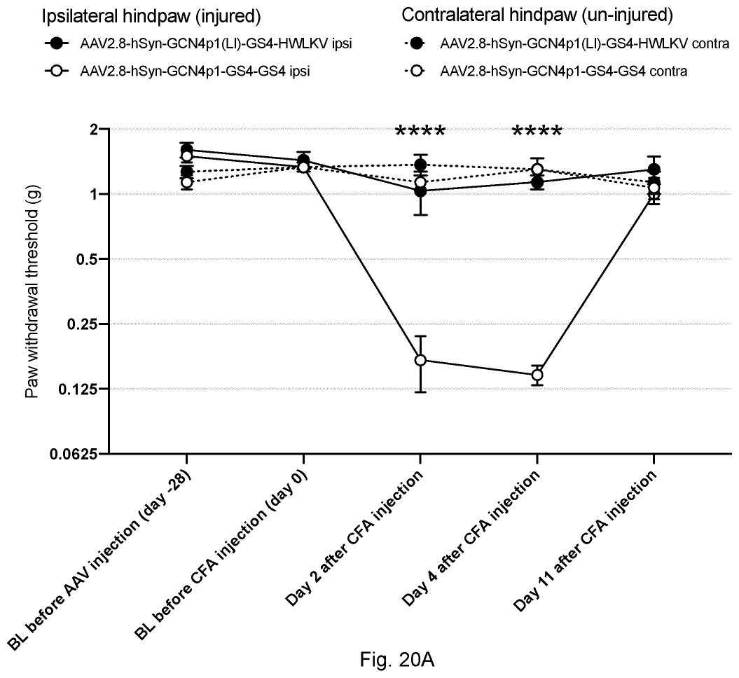

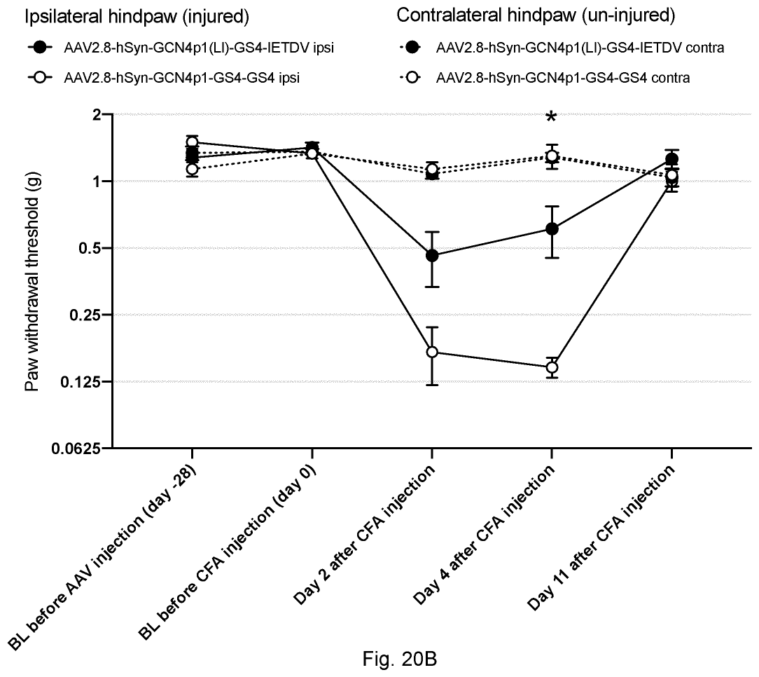

We found that GCN4p1(LI)-GS4-HWLKV, GCN4p1(ILI)-GS4-HWLKV, and CC-Hex2-GS4-HWLKV displayed a superior binding affinity to PICK1 as compared to the dimeric GCN4p1-GS4-HWLKV (FIG. 17A and B). Similarly, GCN4p1(ILI)-GS4-NSVRV and CC-Hex2-GS4-NSVRV displayed a superior binding affinity to PICK1 as compared to the dimeric GCN4p1-GS4-HWLKV (FIG. 17C).

Affinities (Ki) are summarized in the below table, as determined from the ‘One site—Fit’ Ki curve (plot above) for the unlabelled peptides calculated in GrapPad Prism 8.3.

| Polypeptide | Affinity (Kᵢ) (nM) |
|---|---|
| Dimeric GCN4p1-GS4-HWLKV | 297 ± 47 |
| GCN4p1(LI)-GS4-HWLKV | 26.1 ± 4.6 |
| GCN4p1(ILI)-GS4-HWLKV | 1.24 ± 0.2 |
| CC-Tet-GS4-HWLKV | 2.47 ± 0.4 |
| CC-Hex2-GS4-HWLKV | 22.9 ± 3.7 |
| Dimeric GCN4p1-GS4-NSVRV | 63.2 ± 9.2 |
| GCN4p1(LI)-GS4-NSVRV | 16.1 ± 2.8 |
| GCN4p1(ILI)-GS4-NSVRV | 6.14 ± 1.1 |

PSD-95

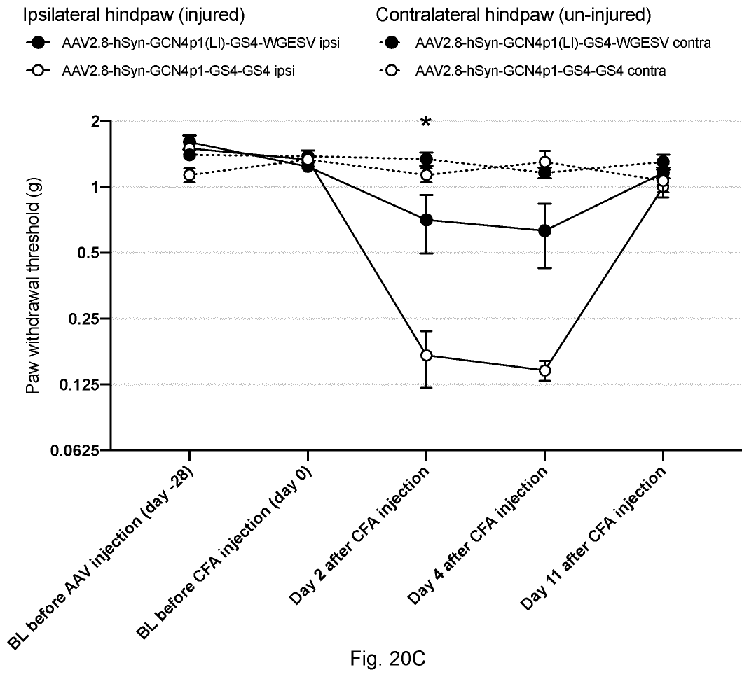

We found that GCN4p1(LI)-GS4-IETDV, GCN4p1(IL1)-GS4-IETDV, and CC-Hex2-GS4-IETDV displayed high binding affinity towards PSD95 PDZ1-2 (FIG. 18A) in line with the affinities for the full length protein thus suggesting an overall enhanced affinity of the higher oligomers as also demonstrated in Example 5. Similarly, GCN4p1(LI)-GS4-RRTTPV, GCN4p1(ILI)-GS4-RRTTPV and CC-Hex2-GS4-RRTTPV displayed high binding affinity towards PSD95 (FIG. 18B).

Affinities (Ki) are summarized in the below table, as determined from the ‘One site—Fit’ Kᵢ curve (plot above) for the unlabelled peptides calculated in GrapPad Prism 8.3.

| Polypeptide | Affinity (Kᵢ) (nM) |
|---|---|
| GCN4p1(LI)-GS4-IETDV | 12.8 ± 1.4 |
| GCN4p1(ILI)-GS4-IETDV | 106 ± 15 |
| CC-Hex2-GS4-IETDV | 56.0 ± 8.2 |
| GCN4p1(LI)-GS4-RRTTPV | 22.8 ± 2.5 |
| GCN4p1(ILI)-GS4-RRTTPV | 111 ± 12 |
| CC-Hex2-GS4-RRTTPV | 242 ± 26 |

Conclusion

In conclusion, this example demonstrates that higher order oligomers of PDZ domains binding motifs (PBM) provide higher affinity towards the PDZ-domain containing proteins, as compared to the dimeric constructs. Furthermore, it is demonstrated that binding of the higher order oligomers of PBMs to the proteins result in formation of higher order oligomers of the respective proteins, an effect which is not observed for the dimeric constructs.

In addition, this example demonstrates that the oligomerization domain may be varied and that the nature of said oligomerization domain is not important for the function of the peptide construct, as long as it provides for higher order oligomers of the PBMs.

### Example 9: Target Engagement Between Various Multivalent PDZ Targeting Peptides and PDZ Proteins

The aim of this series of pull-down experiments was to confirm target engagement between various oligomeric peptide constructs and PDZ-domain containing proteins in lysate from mouse spinal cord tissue.

Materials and Methods:

Peptides

All peptides (95%>purify, and validated by HPLC and Mass Spec analysis) were ordered from TAG Copenhagen A/S and tagged: N-terminal Biotin-Ahx (6-Aminohexanoic acid).

The following PSD-95 targeting peptides were used:

GCN4p1(LI)-GS4-IETDV (SEQ ID NO: 77):
Biotin-Ahx-RIKQIEDKIEEILSKIYHIENEIARIKKLI-GGGGS-
IETDV
GCN4p1(ILI)-GS4-IETDV (SEQ ID NO: 150):
Biotin-Ahx-RMKQIEDKLEEILSKLYHIENELARIKKLL-GGGGS-
IETDV
ccHex2-GS4-IETDV (SEQ ID NO: 95):
Biotin-Ahx-GEIAKSLKEIAKSLKEIAWSLKEIAKSLK-GGGGS-
IETDV

The following PICK1 targeting peptides were used:

GCN4p1(LI)-GS4-HWLKV (SEQ ID NO: 81):
Biotin-Ahx-RIKQIEDKIEEILSKIYHIENEIARIKKLI-GGGGS-
HWLKV
GCN4p1(ILI)-GS4-HWLKV (SEQ ID NO: 148):
Biotin-Ahx-RMKQIEDKLEEILSKLYHIENELARIKKLL-GGGGS-
HWLKV
ccHex2-GS4-HWLKV (SEQ ID NO: 93):
Biotin-Ahx-GEIAKSLKEIAKSLKEIAWSLKEIAKSLK-GGGGS-
HWLKV

The following nNOS targeting peptides were used:

GCN4p1(LI)-GS4-WGESV (SEQ ID NO: 86):
Biotin-Ahx-RIKQIEDKIEEILSKIYHIENEIARIKKLI-GGGGS-
WGESV
GCN4p1(ILI)-GS4-WGESV (SEQ ID NO: 152):
Biotin-Ahx-RMKQIEDKLEEILSKLYHIENELARIKKLL-GGGGS-
WGESV
ccHex-GS4-WGESV (SEQ ID NO: 98):
Biotin-Ahx-GEIAKSLKEIAKSLKEIAWSLKEIAKSLK-GGGGS-
WGESV

The following non-binding control peptide was used:

GCN4p1-GS4-GS4 (SEQ ID NO: 153):
Biotin-Ahx-RMKQLEDKVEELLSKNYHLENEVARLKKLV-GGGGS-
GGGGS

Spinal cord lumbar tract total lysates preparation Spinal cord lysates were prepared from 8 weeks old C57BL/6 mice. Once sacrificed, the spinal cords were immediately dissected in ice-cold PBS1X by hydraulic extrusion according to the procedure described in Richner et al., 2017. The lumbar tract of the spinal cords were quickly harvested and lysed in lysis buffer (50 mM Tris Ph 7.4, 150Mm NaCl, 0.1% SDS, 0.5% NaDeoxycholate, 1% Triton X-100, 5 mM NaF and 1× Roche protease inhibitor cocktail), and the supernatant was collected following centrifugation at 20,000 g for 30 minutes. Lysates were pre-cleared by incubation with streptavidin beads for 1 hour at 4 degrees, and cleared supernatant were transferred to new tubes and stored at −80 degrees before further use.

Pull-Down

For each condition, 30 μL Streptavidin biotin beads (Invitrogen, Dynabeads™ MyOne™ Streptavidin T1; #65601) were washed before incubation with indicated biotinylated peptides for 3 hours at 4 degrees and excess peptides was removed with three washes. 500 pg of pre-cleared lysates were added to the peptide-bound beads and incubated over-night at 4 degrees before three washes and elution in 25 μL SDS loading buffer.

Western Blotting

Samples were separated by SDS-PAGE (BioRad Mini-Protean TGX precast gels; cat #4561084) and transferred to nitrocellulose blot (BioRad Transfer-Blot Turbo Transfer Pack; cat #1704156) and BioRad Turbo Transfer System). Western blots were incubated over-night with primary antibodies as indicated: PICK1 antibody (rabbit), Abcam, Ab3420, LN: GR3324059-5; PICK1 antibody (mouse), monoclonal clone 2G10 custom generated; PSD-95 antibody (mouse), Abcam, ab19275 [K28/43], LN: GR3333330-2; nNOS antibody (rabbit), Abcam, Ab7606, LN:GR315913-19), and after three washes incubated for 1 hour with matching secondary HRP conjugated antibodies (goat anti-rabbit-HRP conj., Pierce, 31402, LN: FB788514) or (goat anti-mouse-HRP conj., ThermoScientific, 31430, LN: MJ163550) before development with ECL signal solution (ThermoScientific Pierce ECL Plus Western Blotting Substrate, ECL Plus Western Blotting Substrate, Cat #32132) before visualization. Images were processed in ImageJ.

Results:

Pull-down experiment with PICK1 binding peptides confirms target engagement with PICK1 protein, whereas the control peptide (biotin-Ahx-GCN4p1-GS4-GS4) does not bind PICK1 (FIG. 19A). Pull-down experiment with PSD-95 binding peptides confirms target engagement with PSD-95 protein, whereas the control peptide (biotin-Ahx-GCN4p1-GS4-GS4) does not bind PSD-95 protein (FIG. 19B). Pull-down experiment with nNOS binding peptides confirms target engagement with nNOS protein, whereas the control peptide (biotin-Ahx-GCN4p1-GS4-GS4) does not bind nNOS protein (FIG. 19C). Pull-down experiment confirming high degree of selective target engagement specified by their PDZ binding motif (HWLKV binds PICK1; IETDV binds PSD-95; WGESV binds nNOS) respectively. The control peptide (biotin-Ahx-GCN4p1-GS4-GS4) does not bind any of the PDZ proteins (FIG. 19D).

Conclusion:

This series of experiments demonstrate high-degree of selective target engagement for all oligomeric peptide tested as specified by their respective type I, II and III PDZ binding motifs.

### Example 10: Efficacy Study of AAV-Encoding GCN4p1 Trimeric Peptide Variants in the CFA Model of Inflammatory Pain

The Complete Freund's Adjuvant (CFA) model of inflammatory pain was used to evaluate pain relief induced by administration of AAV encoding trimeric peptide variants (i.e. LI variants) against PSD-95, PICK1 and nNOS, respectively, in mice.

Materials and Methods:

Pain assessment was made using von Frey measurements at different time points. The mice were administered a single intrathecal injection of either AAV2.8-hSyn-HA-GCN4p1(LI)-GS4-IETDV-WPREpA, AAV2.8-hSyn-HA-GCN4p1(LI)-GS4-HWLKV-WPREpA, or AAV2.8-hSyn-HA-GCN4p1(LI)-GS4-WGESV-WPREpA. The vector AAV2.8-hSyn-HA-GCN4p1-GS4-GS4-WPREpA served as a control.

Virus Made and Tested In Vivo

The tested PDZ-targeting AAV vectors were identical except for their C-terminal C5 PDZ binding domain (XXXXX). The vectors were constructed and manufactured with the following elements: AAV-2.8-hSyn-HA-GCN4p1(LI)-GS4-XXXXX;

Peptide encoding GCN4p1 variants (all starting with M as start codon)

PSD-95 targeting peptide: HA-GCN4p1(LI)-GS4-IETDV
(SEQ ID NO: 155)
MYPYDVPDYA-GS-RIKQIEDKIEEILSKIYHIENEIARIKKLI-
GGGGS-IETDV
PICK1 targeting peptide: HA-GCN4p1(LI)-GS4-HWLKV
(SEQ ID NO: 156)
MYPYDVPDYA-GS-RIKQIEDKIEEILSKIYHIENEIARIKKLI-
GGGGS-HWLKV
nNOS targeting peptide: HA-GCN4p1(LI)-GS4-WGESV
(SEQ ID NO: 157)
MYPYDVPDYA-GS-RIKQIEDKIEEILSKIYHIENEIARIKKLI-
GGGGS-WGESV
Non-binding control: HA-GCN4p1-GS4-GS4
(SEQ ID NO: 158)
MYPYDVPDYA-GS-RMKQLEDKVEELLSKNYHLENEVARLKKLV-
GGGGS-GGGGS
(This control AAV expresses the native dimeric
scaffold, GCN4p1 (i.e. without mutations) and
another GS4 motif at the C-terminus instead of
a PBM, and hence does not bind either PSD-95,
PICK1, or nNOS (Example 9).

Plasmid design. The DNA region spanning the entire coding sequence of HA-GCN4p1(LI)-GS4-IETDV, HA-GCN4p1(LI)-GS4-HWLKV, HA-GCN4p1(LI)-GS4-WGESV, and HA-GCN4p1-GS4-GS4 peptides with appropriate 5′ and 3′ restriction sites were ordered as pre-manufactured circular plasmids, pEX, from Eurofins Genomics. These DNA inserts were next by traditional “cut and paste” restriction enzyme cloning technique inserted into a generic AAV plasmid backbone. This AAV plasmid backbone contained an upstream human Synapsin1 (pan-neuronal) promoter, followed by a multiple cloning site (MCS, containing similar restriction sites as found in the flanking region of the peptide DNA sequences), and terminated by WPRE and Poly A signal. The entire DNA sequence within the AAV plasmid backbone was flanked by the 5″- and 3″-ITRs. Correct insertion and integrity of the final AAV plasmids were confirmed by PCR sequencing.

Viral production. All AAV viruses were generated in-house using a FuGene6 mediated triple plasmid co-transfection method in HEK293FT cells. These procedures have been described earlier (Sørensen et al., 2016, eLife). For the triple transfection, AAV pHelper plasmid, AAV Rep(2)-Cap(8) plasmid and the generated AAV plasmid vectors were used. Three days after transfection, cells were harvested and virus was purified using an adapted Iodixanol gradient purification protocol. Genomic AAV titer was determined by a PicoGreen-based method. Before use, all viruses were carefully examined in Western Blots for purification, and, if needed, diluted in Dulbecco's Phosphate-Buffered Saline (DPBS) for optimized titer.

Animals; 6-10male C57BL6/N mice (SPF status, Janvier, France) of 8 weeks of age at beginning of experiment were used in each group. Mice were allowed at least 7 days of habituation to our facility before initiation of experiment. Mice were group-housed in IVC-cages in a temperature-controlled room maintained on a 12:12 light:dark cycle (lights on at 6 AM) and allowed access to standard rodent chow and water ad libitum.

Virus administration; Mice were injected with one of the following four viruses; rAAV2.8-hSyn-HA-GCN4p1(LI)-GS4-IETDV-WPREpA, rAAV2.8-hSyn-HA-GCN4p1(LI)-GS4-HWLKV-WPREpA, rAAV2.8-hSyn-HA-GCN4p1(LI)-GS4-WGESV-WPREpA, or the control virus; rAAV2.8-hSyn-GCN4p1-GS4-GS4-WPREpA. Each of the four viruses were pre-diluted in DPBS for a final titer of 2.2E+12 vg/ml prior to injection. The virus was delivered by a single intrathecal administration in a volume of 7 μL to mice under isofluorane anesthesia using a 10 μL Hamilton syringe and 30G, 20 mm long, 11 angle tip needle in the intervertebral space between L5/L6 four weeks prior to the von Frey test. The correct position of the needle was assured by a typical flick of the tail.

Induction of inflammatory pain. On day 28 after virus injection, inflammatory pain was induced by the use of Complete Freund's adjuvant (CFA). Mice were placed under very light isoflurane anesthesia. The right hindpaw of the mice was sterilized with ethanol, and 5 μL of CFA was injected intraplantar to the right hindpaw with an insulin needle. Mice woke up within seconds of being removed from the isoflurane, and were left for 48 hours while inflammatory pain developed. The development and level of mechanical hyperalgesia/allodynia was determined in the affected hind paws 2, 4 and 11 days after the CFA procedure by using Von Frey filaments ranging from 0.04 to 2 g. The filaments are applied to the underside of the paw after the mouse has settled into a comfortable position within a restricted area that has a perforated floor. The filaments are calibrated to flex when the set force is applied to the paw. Filaments are presented in order of increasing stiffness, until a paw withdrawal is detected. In the current experiments filaments in ascending order were applied to the central part of the hind paws. Each Von Frey hair was applied five times over a total period of 30 seconds and the mouse's reaction was assessed after each application; the threshold for a positive test was set at 3 trials, which evoked responses out of a maximum of 5 trials. A positive pain reaction is defined as sudden paw withdrawal, flinching and/or paw licking induced by the filament. The non-injected left hindpaw was used as an unaffected control.

Results:

The pain threshold for all treatment groups when measured before virus injection, before CFA injection and at day 11 after CFA injection were all similar (no significant difference between groups; no significant difference between ipsi- and contralateral paw within groups). At day 11 after CFA injection, the pain model reverses, and the pain threshold return to previous baseline values (FIG. 20A-C).

Conclusion

In vivo administration of AAV vectors encoding recombinant GCN4p1(LI) peptides aimed at targeting class I, II and III PDZ domain proteins, respectively, induces pain relief in the CFA model of inflammatory pain.

Sequences

| | | |
|---|---|---|
| SEQ ID NO: | Sequence | Comment |

Peptide sequences

| | | |
|---|---|---|
| SEQ ID NO: 1 | X₁X₂X₃X₄X₅, wherein: | Generic DAT- |
| | X₁ is H, N, F, or T, or is absent; | C5 variants |
| | X₂ is W, S, E, or Y; or is absent; | |
| | X₃ is L, V, or I; | |
| | X₄ is K, I, or R; and | |
| | X₅ is V; | |
| SEQ ID NO: 2 | X₁X₂X₃X₄X₅, wherein: | Generic DAT- |
| | X₁ is N, F, or T, or is absent; | C5 variants |
| | X₂ is S, E, or Y; or is absent; | |
| | X₃ is V, L or I; | |
| | X₄ is I or R; and | |
| | X₅ is V | |
| SEQ ID NO: 3 | X₁X₂X₃X₄X₅, wherein: | Generic DAT- |
| | X₁ is N or T, or is absent; | C5 variants |
| | X₂ is S, E, or Y; or is absent; | |
| | X₃ is V, L or I; | |
| | X₄ is I or R; and | |
| | X₅ is V | |
| SEQ ID NO: 4 | X₁X₂X₃X₄X₅, wherein: | Generic DAT- |
| | X₁ is N or F, or is absent; | C5 variants |
| | X₂ is S, E, or Y; or is absent; | |
| | X₃ is V, L or I; | |
| | X₄ is I or R; and | |
| | X₅ is V | |
| SEQ ID NO: 196 | X₁X₂X₃X₄X₅X₆; | Generic Class |
| | wherein | I C5-C6 |
| | X₁ is Y, R or is absent; | variants |
| | X₂ is R, K, I or is absent; | |
| | X₃ is T, E, Q; or is absent; | |
| | X₄ is T; | |
| | X₅ is D, S or P; and | |
| | X₆ is V. | |
| SEQ ID NO: 5 | NSIIV | DAT-C5 |
| | | variant |
| SEQ ID NO: 6 | NSIRV | DAT-C5 |
| | | variant |
| SEQ ID NO: 7 | NSVIV | DAT-C5 |
| | | variant |
| SEQ ID NO: 8 | NSVRV | DAT-C5 |
| | | variant |
| SEQ ID NO: 9 | NEIIV | DAT-C5 |
| | | variant |
| SEQ ID NO: 10 | NEIRV | DAT-C5 |
| | | variant |
| SEQ ID NO: 11 | NEVIV | DAT-C5 |
| | | variant |
| SEQ ID NO: 12 | NEVRV | DAT-C5 |
| | | variant |
| SEQ ID NO: 13 | NYIIV | DAT-C5 |
| | | variant |
| SEQ ID NO: 14 | NYIRV | DAT-C5 |
| | | variant |
| SEQ ID NO: 15 | NYVIV | DAT-C5 |
| | | variant |
| SEQ ID NO: 16 | NYVRV | DAT-C5 |
| | | variant |
| SEQ ID NO: 17 | TSIIV | DAT-C5 |
| | | variant |
| SEQ ID NO: 18 | TSIRV | DAT-C5 |
| | | variant |
| SEQ ID NO: 19 | TSVIV | DAT-C5 |
| | | variant |
| SEQ ID NO: 20 | TSVRV | DAT-C5 |
| | | variant |
| SEQ ID NO: 21 | TEIIV | DAT-C5 |
| | | variant |
| SEQ ID NO: 22 | TEIRV | DAT-C5 |
| | | variant |
| SEQ ID NO: 23 | TEVIV | DAT-C5 |
| | | variant |
| SEQ ID NO: 24 | TEVRV | DAT-C5 |
| | | variant |
| SEQ ID NO: 25 | TYIIV | DAT-C5 |
| | | variant |
| SEQ ID NO: 26 | TYIRV | DAT-C5 |
| | | variant |
| SEQ ID NO: 27 | TYVIV | DAT-C5 |
| | | variant |
| SEQ ID NO: 28 | TYVRV | DAT-C5 |
| | | variant |
| SEQ ID NO: 29 | FSIIV | DAT-C5 |
| | | variant |
| SEQ ID NO: 30 | FSIRV | DAT-C5 |
| | | variant |
| SEQ ID NO: 31 | FSVIV | DAT-C5 |
| | | variant |
| SEQ ID NO: 32 | FSVRV | DAT-C5 |
| | | variant |
| SEQ ID NO: 33 | FEIIV | DAT-C5 |
| | | variant |
| SEQ ID NO: 34 | FEIRV | DAT-C5 |
| | | variant |
| SEQ ID NO: 35 | FEVIV | DAT-C5 |
| | | variant |
| SEQ ID NO: 36 | FEVRV | DAT-C5 |
| | | variant |
| SEQ ID NO: 37 | FYIIV | DAT-C5 |
| | | variant |
| SEQ ID NO: 38 | FYIRV | DAT-C5 |
| | | variant |
| SEQ ID NO: 39 | FYVIV | DAT-C5 |
| | | variant |
| SEQ ID NO: 40 | FYVRV | DAT-C5 |
| | | variant |
| SEQ ID NO: 41 | SIIV | DAT-C5 |
| | | variant |
| SEQ ID NO: 42 | SIRV | DAT-C5 |
| | | variant |
| SEQ ID NO: 43 | SVIV | DAT-C5 |
| | | variant |
| SEQ ID NO: 44 | SVRV | DAT-C5 |
| | | variant |
| SEQ ID NO: 45 | EIIV | DAT-C5 |
| | | variant |
| SEQ ID NO: 46 | EIRV | DAT-C5 |
| | | variant |
| SEQ ID NO: 47 | EVIV | DAT-C5 |
| | | variant |
| SEQ ID NO: 48 | EVRV | DAT-C5 |
| | | variant |
| SEQ ID NO: 49 | YIIV | DAT-C5 |
| | | variant |
| SEQ ID NO: 50 | YIRV | DAT-C5 |
| | | variant |
| SEQ ID NO: 51 | YVIV | DAT-C5 |
| | | variant |
| SEQ ID NO: 52 | YVRV | DAT-C5 |
| | | variant |
| SEQ ID NO: 53 | NSLRV | DAT-C5 |
| | | variant |
| SEQ ID NO: 54 | HWLKV | DAT-C5 |
| N/A | IIV | DAT-C5 |
| | | variant |
| N/A | IRV | DAT-C5 |
| | | variant |
| N/A | VIV | DAT-C5 |
| | | variant |
| N/A | VRV | DAT-C5 |
| | | variant |
| N/A | LRV | DAT-C5 |
| | | variant |
| SEQ ID NO: 55 | IETDV | Class I |
| SEQ ID NO: 56 | RRTTPV | Class I |
| SEQ ID NO: 57 | YKQTSV | Class I |
| SEQ ID NO: 58 | WGESV | Class III |
| SEQ ID NO: 59 | KVDSV | Class III |
| SEQ ID NO: 60 | GKDYV | Class III |
| SEQ ID NO: 61 | RKDYV | Class III |
| SEQ ID NO: 62 | TAEMF | Class III |
| SEQ ID NO: 63 | QEDGA | Class III |
| SEQ ID NO: 64 | IETAV | Class I |
| SEQ ID NO: 65 | SNTANRRTTPV | RRTTPV |
| | | monomer |
| SEQ ID NO: 66 | RMKQLEDKVEELLSKNYHLENEVARLKKLV | GCN4p1 |
| SEQ ID NO: 67 | RMKQLEDKVEELLSKQYHLENEVARLKKLV | GCN4p1(NQ) |
| SEQ ID NO: 68 | RIKQIEDKIEEILSKIYHIENEIARIKKLI | GCN4p1(LI) |
| SEQ ID NO: 69 | GELAAIKQELAAIKKELAAIKWELAAIKQ | CC-Tet |
| SEQ ID NO: 70 | GEIAKSLKEIAKSLKEIAWSLKEIAKSLK | CC-Hex2 |
| N/A | GGS | GS2 |
| SEQ ID NO: 71 | GGGS | GS3 |
| SEQ ID NO: 72 | GGGGS | GS4 |
| SEQ ID NO: 73 | GGGGSG | GS5 |
| SEQ ID NO: 74 | GGGGSGG | GS6 |
| SEQ ID NO: 75 | RMKQLEDKVEELLSKQYHLENEVARLKKLVGGGGS | GCN4p1(NQ)- |
| | HWLKV | GS4-HWLKV |
| SEQ ID NO: 76 | RMKQLEDKVEELLSKQYHLENEVARLKKLVGGGGS | GCN4p1(NQ)- |
| | NSIRV | GS4-NSIRV |
| SEQ ID NO: 77 | RMKQLEDKVEELLSKQYHLENEVARLKKLVGGGGSI | GCN4p1(NQ)- |
| | ETDV | GS4-IETDV |
| SEQ ID NO: 78 | RMKQLEDKVEELLSKQYHLENEVARLKKLVGGGGS | GCN4p1(NQ)- |
| | RRTTPV | GS4-RRTTPV |
| SEQ ID NO: 79 | RMKQLEDKVEELLSKQYHLENEVARLKKLVGGGGS | GCN4p1(NQ)- |
| | YKQTSV | GS4-YKQTSV |
| SEQ ID NO: 80 | RMKQLEDKVEELLSKQYHLENEVARLKKLVGGGGS | GCN4p1(NQ)- |
| | WGESV | GS4-WGESV |
| SEQ ID NO: 81 | RIKQIEDKIEEILSKIYHIENEIARIKKLIGGGGSHWLKV | GCN4p1(LI)- |
| | | GS4-HWLKV |
| SEQ ID NO: 82 | RIKQIEDKIEEILSKIYHIENEIARIKKLIGGGGSNSIRV | GCN4p1(LI)- |
| | | GS4-NSIRV |
| SEQ ID NO: 83 | RIKQIEDKIEEILSKIYHIENEIARIKKLIGGGGSIETDV | GCN4p1(LI)- |
| | | GS4-IETDV |
| SEQ ID NO: 84 | RIKQIEDKIEEILSKIYHIENEIARIKKLIGGGGSRRTTP | GCN4p1(LI)- |
| | | GS4-RRTTPV |
| SEQ ID NO: 85 | RIKQIEDKIEEILSKIYHIENEIARIKKLIGGGGSYKQTS | GCN4p1(LI)- |
| | V | GS4-YKQTSV |
| SEQ ID NO: 86 | RIKQIEDKIEEILSKIYHIENEIARIKKLIGGGGSWGESV | GCN4p1(LI)- |
| | | GS4-WGESV |
| SEQ ID NO: 87 | GELAAIKQELAAIKKELAAIKWELAAIKQGGGGSHWL | CC-Tet-GS4- |
| | KV | HWLKV |
| SEQ ID NO: 88 | GELAAIKQELAAIKKELAAIKWELAAIKQGGGGSNSIR | CC-Tet-GS4- |
| | V | NSIRV |
| SEQ ID NO: 89 | GELAAIKQELAAIKKELAAIKWELAAIKQGGGGSIETD | CC-Tet-GS4- |
| | V | IETDV |
| SEQ ID NO: 90 | GELAAIKQELAAIKKELAAIKWELAAIKQGGGGSRRTT | CC-Tet-GS4- |
| | PV | RRTTPV |
| SEQ ID NO: 91 | GELAAIKQELAAIKKELAAIKWELAAIKQGGGGSYKQ | CC-Tet-GS4- |
| | TSV | YKQTSV |
| SEQ ID NO: 92 | GELAAIKQELAAIKKELAAIKWELAAIKQGGGGSWGE | CC-Tet-GS4- |
| | SV | WGESV |
| SEQ ID NO: 93 | GEIAKSLKEIAKSLKEIAWSLKEIAKSLKGGGGSHWLK | CC-Hex2- |
| | | GS4-HWLKV |
| SEQ ID NO: 94 | GEIAKSLKEIAKSLKEIAWSLKEIAKSLKGGGGSNSIR | CC-Hex2- |
| | V | GS4-NSIRV |
| SEQ ID NO: 95 | GEIAKSLKEIAKSLKEIAWSLKEIAKSLKGGGGSIETD | CC-Hex2- |
| | V | GS4-IETDV |
| SEQ ID NO: 96 | GEIAKSLKEIAKSLKEIAWSLKEIAKSLKGGGGSRRTT | CC-Hex2- |
| | PV | GS4-RRTTPV |
| SEQ ID NO: 97 | GEIAKSLKEIAKSLKEIAWSLKEIAKSLKGGGGSYKQT | CC-Hex2- |
| | SV | GS4-YKQTSV |
| SEQ ID NO: 98 | GEIAKSLKEIAKSLKEIAWSLKEIAKSLKGGGGSWGE | CC-Hex2- |
| | SV | GS4-WGESV |
| SEQ ID NO: 99 | RMKQLEDKVEELLSKNYHLENEVARLKKLVGGGGS | GCN4p1-GS4- |
| | HWLKV | HWLKV |
| SEQ ID NO: 100 | RMKQLEDKVEELLSKNYHLENEVARLKKLVGGGGS | GCN4p1-GS4- |
| | RRTTPV | RRTTPV |
| SEQ ID NO: 101 | RMKQLEPKVEELLPKNYHLENEVARLKKLVGGGGSR | GCN4p1(7P14P)- |
| | RTTPV | GS4-RRTTPV |
| SEQ ID NO: 102 | RMKQLEDKVEELLSKNYHLENEVARLKKLVGGGGSI | GCN4p1-GS4- |
| | ETDV | IETDV |
| SEQ ID NO: 103 | RMKQLEPKVEELLPKNYHLENEVARLKKLVGGGGSI | GCN4p1(7P14P)- |
| | ETDV | GS4-IETDV |
| SEQ ID NO: 104 | LXXXXXXLXXXXXXLXXXXXXL | Generic coiled |
| | | coil |
| SEQ ID NO: 105 | MXXLXXXVXXLXXXQXXLXXXVXXLXXXV | Generic |
| | | trimeric coiled |
| | | coil |
| SEQ ID NO: 106 | IXXIXXXIXXIXXXIXX XXIXXIXXXI | Generic |
| | | tetrameric |
| | | coiled coil |
| SEQ ID NO: 107 | LXXIXXXLXXIXXXLXXIXXXLXXI | Generic |
| | | tetrameric |
| | | coiled coil |
| SEQ ID NO: 108 | IXXXLXXIXXXLXXIXXXLXXIXXXL | Generic |
| | | hexameric |
| | | coiled coil |
| SEQ ID NO: 144 | RIKQIEDKIEEILSKIYHIENEIARIKKLIGGGGSNSVRV | GCN4p1(LI)- |
| | | GS4-NSVRV |
| SEQ ID NO: 145 | RMKQLEDKVEELLSKNYHLENEVARLKKLVGGGGS | GCN4p1-GS4- |
| | NSVRV | NSVRV |
| SEQ ID NO: 146 | GEIAKSLKEIAKSLKEIAWSLKEIAKSLKGGGGSNSVR | CC-Hex2- |
| | V | GS4-NSVRV |
| SEQ ID NO: 147 | RMKQIEDKLEEILSKLYHIENELARIKKLL | GCN4p1(ILI) |
| SEQ ID NO: 148 | RMKQIEDKLEEILSKLYHIENELARIKKLLGGGGSHWL | GCN4p1(ILI)- |
| | KV | GS4-HWLKV |
| SEQ ID NO: 149 | RMKQIEDKLEEILSKLYHIENELARIKKLLGGGGSNSV | GCN4p1(ILI)- |
| | RV | GS4-NSVRV |
| SEQ ID NO: 150 | RMKQIEDKLEEILSKLYHIENELARIKKLLGGGGSIETD | GCN4p1(ILI)- |
| | V | GS4-IETDV |
| SEQ ID NO: 151 | RMKQIEDKLEEILSKLYHIENELARIKKLLGGGGSRRT | GCN4p1(ILI)- |
| | TPV | GS4-RRTTPV |
| SEQ ID NO: 152 | RMKQIEDKLEEILSKLYHIENELARIKKLLGGGGSWG | GCN4p1(ILI)- |
| | ESV | GS4-WGESV |
| SEQ ID NO: 153 | RMKQLEDKVEELLSKNYHLENEVARLKKLVGGGGS | GCN4p1-GS4- |
| | GGGGS | GS4 |
| SEQ ID NO: 154 | VSSIEKKIEEITSQIIQISNEITLIRNEIAQIKQ | ATF7-pII |
| SEQ ID NO: 155 | MYPYDVPDYAGSRIKQIEDKIEEILSKIYHIENEIARIKK | HA- |
| | LIGGGGSIETDV | GCN4p1(LI)- |
| | | GS4-IETDV |
| SEQ ID NO: 156 | MYPYDVPDYAGSRIKQIEDKIEEILSKIYHIENEIARIKK | HA- |
| | LIGGGGSHWLKV | GCN4p1(LI)- |
| | | GS4-HWLKV |
| SEQ ID NO: 157 | MYPYDVPDYAGSRIKQIEDKIEEILSKIYHIENEIARIKK | HA- |
| | LIGGGGSWGESV | GCN4p1(LI)- |
| | | GS4-WGESV |
| SEQ ID NO: 158 | MYPYDVPDYAGSRMKQLEDKVEELLSKNYHLENEV | HA-GCN4p1- |
| | ARLKKLVGGGGSGGGGS | GS4-GS4 |
| SEQ ID NO: 159 | VQSIEKKIEDISSLIGQIQSEITLIRNEIAQIKQ | ATF2-pII |
| SEQ ID NO: 160 | DRAVIKEISEKIELIEKAIASIQLQIDEIKQTIAKIEED | NRP-pII |
| SEQ ID NO: 161 | EEKSLVDTIYAIKDEIQEIRQDNKKIKKSIEEIQRAIKDL | PIX-pII |
| | EKLIRKI | |
| SEQ ID NO: 162 | EKEISAIRQEIADIRKEIGKIKNIIAKIEARHG | HLF-pII |
| SEQ ID NO: 163 | EKEIALIRQEIVAIRQEISHIRAVISRIQAQHG | DBP-pII |
| SEQ ID NO: 164 | EKEITAIRTEIAEIRKEIGKIKTIISKIETK | TEF-pII |
| SEQ ID NO: 165 | SPSELDISKISHKIKEIQIKIAVIEAEIQKIKTKIQAIENEK | NRBI-pII |
| SEQ ID NO: 166 | ESRIAAISAQIQEIQKKIQEIERHIISIVAQIRQIQ | CREB4-pII |
| SEQ ID NO: 167 | ETRISAITAQIQEIQRKILHIEKQILSILEQIKKIQ | CREBH-pII |
| SEQ ID NO: 168 | EKHDQCKCENLIMFQNLANEEIRKITQRIEEITQRIEAI | MAT2-pII |
| | ENR | |
| SEQ ID NO: 194 | VSSIEKKIEEITSQIIQISNEITLIRNEIAQIKQGGGGSH | ATF7-GS4- |
| | WLKV | HWLKV |
| SEQ ID NO: 195 | VSSIEKKIEEITSQIIQISNEITLIRNEIAQIKQGGGGSIE | ATF7-GS4- |
| | TDV | IETDV |

DNA sequences

| | | |
|---|---|---|
| SEQ ID NO: 109 | atgcgcatgaaacagctggaagataaagtggaagaactgctgagcaaa | GCN4p1(NQ) |
| | cagtatcatctggaaaacgaagtggcgcgcctgaaaaaactggtg | |
| SEQ ID NO: 110 | atgcgcatcaaacagatcgaagataaaatcgaagaaatcctgagcaaa | GCN4p1(LI) |
| | atctatcatatcgaaaacgaaatcgcgcgcatcaaaaaactgatc | |
| SEQ ID NO: 111 | ggcgagctggccgccatcaagcaggagctggccgccatcaagaagga | CC-Tet |
| | gctggccgccatcaagtgggagctggccgccatcaagcag | |
| SEQ ID NO: 112 | ggcgagatcgccaagagcctgaaggagatcgccaagagcctgaagga | CC-Hex2 |
| | gatcgcctggagcctgaaggagatcgccaagagcctgaag | |
| SEQ ID NO: 113 | cattggctgaaagtg | HWLKV |
| SEQ ID NO: 114 | aacagcatcagggtg | NSIRV |
| SEQ ID NO: 115 | atcgagaccgacgtg | IETDV |
| SEQ ID NO: 116 | aggaggaccacccccgtg | RRTTPV |
| SEQ ID NO: 117 | tacaagcagaccagcgtg | YKQTSV |
| SEQ ID NO: 118 | tggggcgagagcgtg | WGESV |
| SEQ ID NO: 119 | ggcggcggcggcagc | GS4 |
| SEQ ID NO: 120 | cgcatgaaacagctggaagataaagtggaagaactgctgagcaaaca | GCN4p1(NQ)- |
| | gtatcatctggaaaacgaagtggcgcgcctgaaaaaactggtgggcgg | GS4-HWLKV |
| | cggcggcagccattggctgaaagtg | |
| SEQ ID NO: 121 | cgcatgaaacagctggaagataaagtggaagaactgctgagcaaaca | GCN4p1(NQ)- |
| | gtatcatctggaaaacgaagtggcgcgcctgaaaaaactggtgggcgg | GS4-NSIRV |
| | cggcggcagcaacagcatcagggtg | |
| SEQ ID NO: 122 | cgcatgaaacagctggaagataaagtggaagaactgctgagcaaaca | GCN4p1(NQ)- |
| | gtatcatctggaaaacgaagtggcgcgcctgaaaaaactggtgggcgg | GS4-IETDV |
| | cggcggcagcatcgagaccgacgtg | |
| SEQ ID NO: 123 | cgcatgaaacagctggaagataaagtggaagaactgctgagcaaaca | GCN4p1(NQ)- |
| | gtatcatctggaaaacgaagtggcgcgcctgaaaaaactggtgggcgg | GS4-RRTTPV |
| | cggcggcagcaggaggaccacccccgtg | |
| SEQ ID NO: 124 | cgcatgaaacagctggaagataaagtggaagaactgctgagcaaaca | GCN4p1(NQ)- |
| | gtatcatctggaaaacgaagtggcgcgcctgaaaaaactggtgggcgg | GS4-YKQTSV |
| | cggcggcagctacaagcagaccagcgtg | |
| SEQ ID NO: 125 | cgcatgaaacagctggaagataaagtggaagaactgctgagcaaaca | GCN4p1(NQ)- |
| | gtatcatctggaaaacgaagtggcgcgcctgaaaaaactggtgggcgg | GS4-WGESV |
| | cggcggcagctggggcgagagcgtg | |
| SEQ ID NO: 126 | cgcatcaaacagatcgaagataaaatcgaagaaatcctgagcaaaatc | GCN4p1(LI)- |
| | tatcatatcgaaaacgaaatcgcgcgcatcaaaaaactgatcggcggcg | GS4-HWLKV |
| | gcggcagccattggctgaaagtg | |
| SEQ ID NO: 127 | cgcatcaaacagatcgaagataaaatcgaagaaatcctgagcaaaatc | GCN4p1(LI)- |
| | tatcatatcgaaaacgaaatcgcgcgcatcaaaaaactgatcggcggcg | GS4-NSIRV |
| | gcggcagcaacagcatcagggtg | |
| SEQ ID NO: 128 | cgcatcaaacagatcgaagataaaatcgaagaaatcctgagcaaaatc | GCN4p1(LI)- |
| | tatcatatcgaaaacgaaatcgcgcgcatcaaaaaactgatcggcggcg | GS4-IETDV |
| | gcggcagcatcgagaccgacgtg | |
| SEQ ID NO: 129 | cgcatcaaacagatcgaagataaaatcgaagaaatcctgagcaaaatc | GCN4p1(LI)- |
| | tatcatatcgaaaacgaaatcgcgcgcatcaaaaaactgatcggcggcg | GS4-RRTTPV |
| | gcggcagcaggaggaccacccccgtg | |
| SEQ ID NO: 130 | cgcatcaaacagatcgaagataaaatcgaagaaatcctgagcaaaatc | GCN4p1(LI)- |
| | tatcatatcgaaaacgaaatcgcgcgcatcaaaaaactgatcggcggcg | GS4-YKQTSV |
| | goggcagctacaagcagaccagcgtg | |
| SEQ ID NO: 131 | cgcatcaaacagatcgaagataaaatcgaagaaatcctgagcaaaatc | GCN4p1(LI)- |
| | tatcatatcgaaaacgaaatcgcgcgcatcaaaaaactgatcggcggcg | GS4-WGESV |
| | gcggcagctggggcgagagcgtg | |
| SEQ ID NO: 132 | ggcgagctggccgccatcaagcaggagctggccgccatcaagaagga | CC-Tet-GS4- |
| | gctggccgccatcaagtgggagctggccgccatcaagcagggcggcg | HWLKV |
| | gcggcagccactggctgaaggtg | |
| SEQ ID NO: 133 | ggcgagctggccgccatcaagcaggagctggccgccatcaagaagga | CC-Tet-GS4- |
| | gctggccgccatcaagtgggagctggccgccatcaagcagggcggcg | NSIRV |
| | goggcagcaacagcatcagggtg | |
| SEQ ID NO: 134 | ggcgagctggccgccatcaagcaggagctggccgccatcaagaagga | CC-Tet-GS4- |
| | gctggccgccatcaagtgggagctggccgccatcaagcagggcggcg | IETDV |
| | gcggcagcatcgagaccgacgtg | |
| SEQ ID NO: 135 | ggcgagctggccgccatcaagcaggagctggccgccatcaagaagga | CC-Tet-GS4- |
| | gctggccgccatcaagtgggagctggccgccatcaagcagggcggcg | RRTTPV |
| | gcggcagcaggaggaccacccccgtg | |
| SEQ ID NO: 136 | ggcgagctggccgccatcaagcaggagctggccgccatcaagaagga | CC-Tet-GS4- |
| | gctggccgccatcaagtgggagctggccgccatcaagcagggcggcg | YKQTSV |
| | gcggcagctacaagcagaccagcgtg | |
| SEQ ID NO: 137 | ggcgagctggccgccatcaagcaggagctggccgccatcaagaagga | CC-Tet-GS4- |
| | gctggccgccatcaagtgggagctggccgccatcaagcagggcggcg | WGESV |
| | gcggcagctggggcgagagcgtg | |
| SEQ ID NO: 138 | ggcgagatcgccaagagcctgaaggagatcgccaagagcctgaagga | CC-Hex2- |
| | gatcgcctggagcctgaaggagatcgccaagagcctgaagggcggcg | GS4-HWLKV |
| | gcggcagccactggctgaaggtg | |
| SEQ ID NO: 139 | ggcgagatcgccaagagcctgaaggagatcgccaagagcctgaagga | CC-Hex2- |
| | gatcgcctggagcctgaaggagatcgccaagagcctgaagggcggcg | GS4-NSIRV |
| | gcggcagcaacagcatcagggtg | |
| SEQ ID NO: 140 | ggcgagatcgccaagagcctgaaggagatcgccaagagcctgaagga | CC-Hex2- |
| | gatcgcctggagcctgaaggagatcgccaagagcctgaagggcggcg | GS4-IETDV |
| | gcggcagcatcgagaccgacgtg | |
| SEQ ID NO: 141 | ggcgagatcgccaagagcctgaaggagatcgccaagagcctgaagga | CC-Hex2- |
| | gatcgcctggagcctgaaggagatcgccaagagcctgaagggcggcg | GS4-RRTTPV |
| | gcggcagcaggaggaccacccccgtg | |
| SEQ ID NO: 142 | ggcgagatcgccaagagcctgaaggagatcgccaagagcctgaagga | CC-Hex2- |
| | gatcgcctggagcctgaaggagatcgccaagagcctgaagggcggcg | GS4-YKQTSV |
| | gcggcagctacaagcagaccagcgtg | |
| SEQ ID NO: 143 | ggcgagatcgccaagagcctgaaggagatcgccaagagcctgaagga | CC-Hex2- |
| | gatcgcctggagcctgaaggagatcgccaagagcctgaagggcggcg | GS4-WGESV |
| | gcggcagctggggcgagagcgtg | |
| SEQ ID NO: 169 | cgcatcaaacagatogaagataaaatcgaagaaatcctgagcaaaatc | GCN4p1(LI)- |
| | tatcatatcgaaaacgaaatcgcgcgcatcaaaaaactgatcggcggcg | GS4-NSVRV |
| | gcggcagcaacagcgtgagggtgaacagcgtgagggtg | |
| SEQ ID NO: 170 | agaatgaagcagctggaggacaaggtggaggagctgctgagcaagaa | GCN4p1-GS4- |
| | ctaccacctggagaacgaggtggccagactgaagaagctggtgggcgg | NSVRV |
| | cggcggcagcatcaacagcgtgagggtg | |
| SEQ ID NO: 171 | cgcatgaaacagctggaagataaagtggaagaactgctgagcaaaca | GCN4p1(NQ)- |
| | gtatcatctggaaaacgaagtggcgcgcctgaaaaaactggtgggcgg | GS4-NSVRV |
| | cggcggcagcaacagcgtgagggtg | |
| SEQ ID NO: 172 | ggcgagatcgccaagagcctgaaggagatcgccaagagcctgaagga | CC-Hex2- |
| | gatcgcctggagcctgaaggagatcgccaagagcctgaagggcggcg | GS4-NSVRV |
| | gcggcagc | |
| SEQ ID NO: 173 | cgcatgaaacagatcgaagataaactggaagaaatcctgagcaaactg | GCN4p1(ILI) |
| | tatcatatcgaaaacgaactggcgcgcatcaaaaaactggtg | |
| SEQ ID NO: 174 | cgcatgaaacagatcgaagataaactggaagaaatcctgagcaaactg | GCN4p1(ILI)- |
| | tatcatatcgaaaacgaactggcgcgcatcaaaaaactggtgggcggcg | GS4-HWLKV |
| | gcggcagccattggctgaaagtg | |
| SEQ ID NO: 175 | cgcatgaaacagatcgaagataaactggaagaaatcctgagcaaactg | GCN4p1(ILI)- |
| | tatcatatcgaaaacgaactggcgcgcatcaaaaaactggtgggcggcg | GS4-NSVRV |
| | gcggcagcaacagcgtgagggtg | |
| SEQ ID NO: 176 | cgcatgaaacagatcgaagataaactggaagaaatcctgagcaaactg | GCN4p1(ILI)- |
| | tatcatatcgaaaacgaactggcgcgcatcaaaaaactggtgggcggcg | GS4-IETDV |
| | gcggcagcatcgagaccgacgtg | |
| SEQ ID NO: 177 | cgcatgaaacagatcgaagataaactggaagaaatcctgagcaaactg | GCN4p1(ILI)- |
| | tatcatatcgaaaacgaactggcgcgcatcaaaaaactggtgggcggcg | GS4-RRTTPV |
| | gcggcagcaggaggaccacccccgtg | |
| SEQ ID NO: 178 | cgcatgaaacagatcgaagataaactggaagaaatcctgagcaaactg | GCN4p1(ILI)- |
| | tatcatatcgaaaacgaactggcgcgcatcaaaaaactggtgggcggcg | GS4-WGESV |
| | gcggcagctggggcgagagcgtg | |
| SEQ ID NO: 179 | cgcatgaaacagctggaagataaagtggaagaactgctgagcaaaaa | GCN4p1-GS4- |
| | ctatcatctggaaaacgaagtggcgcgcctgaaaaaactggtgggcggc | GS4 (non- |
| | ggcggcagcggcggcggcggcagc | binding) |
| SEQ ID NO: 180 | gtgagcagcatcgagaagaagatcgaggagatcaccagccagatcatc | ATF7-pII |
| | cagatcagcaacgagatcaccctgatcagaaacgagatcgcccagatc | |
| | aagcag | |
| SEQ ID NO: 181 | aacagcgtgagggtg | NSVRV |
| SEQ ID NO: 182 | gtgcagagcatcgagaagaagatcgaggacatcagcagcctgatcggc | ATF2-pII |
| | cagatccagagcgagatcaccctgatcagaaacgagatcgcccagatc | |
| | aagcag | |
| SEQ ID NO: 183 | gacagagccgtgatcaaggagatcagcgagaagatcgagctgatcga | NRP-pII |
| | gaaggccatcgccagcatccagctgcagatcgacgagatcaagcaga | |
| | ccatcgccaagatcgaggaggac | |
| SEQ ID NO: 184 | gaggagaagagcctggtggacaccatctacgccatcaaggacgagatc | PIX-pII |
| | caggagatcagacaggacaacaagaagatcaagaagagcatcgagg | |
| | agatccagagagccatcaaggacctggagaagctgatcagaaagatc | |
| SEQ ID NO: 185 | gagaaggagatcagcgccatcagacaggagatcgccgacatcagaaa | HLF-pII |
| | ggagatcggcaagatcaagaacatcatcgccaagatcgaggccagac | |
| | acggc | |
| SEQ ID NO: 186 | gagaaggagatcgccctgatcagacaggagatcgtggccatcagacag | DBP-pII |
| | gagatcagccacatcagagccgtgatcagcagaatccaggcccagcac | |
| | ggc | |
| SEQ ID NO: 187 | gagaaggagatcaccgccatcagaaccgagatcgccgagatcagaaa | TEF-pII |
| | ggagatcggcaagatcaagaccatcatcagcaagatcgagaccaag | |
| SEQ ID NO: 188 | agccccagcgagctggacatcagcaagatcagccacaagatcaagga | NRBI-pII |
| | gatccagatcaagatcgccgtgatcgaggccgagatccagaagatcaa | |
| | gaccaagatccaggccatcgagaacgagaag | |
| SEQ ID NO: 189 | gagagcagaatcgccgccatcagcgcccagatccaggagatccagaa | CREB4-pII |
| | gaagatccaggagatcgagagacacatcatcagcatcgtggcccagat | |
| | cagacagatccag | |
| SEQ ID NO: 190 | gagaccagaatcagcgccatcaccgcccagatccaggagatccagag | CREBH-pII |
| | aaagatcctgcacatcgagaagcagatcctgagcatcctggagcagatc | |
| | aagaagatccag | |
| SEQ ID NO: 191 | gagaagcacgaccagtgcaagtgcgagaacctgatcatgttccagaac | MAT2-pII |
| | ctggccaacgaggagatcagaaagatcacccagagaatcgaggagat | |
| | cacccagagaatcgaggccatcgagaacaga | |
| SEQ ID NO: 192 | gtgagcagcatcgagaagaagatcgaggagatcaccagccagatcatc | ATF7-GS4- |
| | cagatcagcaacgagatcaccctgatcagaaacgagatcgcccagatc | HWLKV |
| | aagcagggcggcggcggcagccattggctgaaagtg | |
| SEQ ID NO: 193 | gtgagcagcatcgagaagaagatcgaggagatcaccagccagatcatc | ATF7-GS4- |
| | cagatcagcaacgagatcaccctgatcagaaacgagatcgcccagatc | IETDV |
| | aagcagggcggcggcggcagcatcgagaccgacgtg | |

### REFERENCES

ALFONSO, S., et al., Synapto-depressive effects of amyloid beta require PICK1. Eur J Neurosci, 2014. 39(7): p. 1225-33.
ATIANJOH, F. E., et al., Spinal cord protein interacting with C kinase 1 is required for the maintenance of complete Freund's adjuvant-induced inflammatory pain but not for incision-induced post-operative pain. Pain, 2010. 151(1): p. 226-34.
BACH et al., PNAS, 109(9):3317-22, 2012
CHEN, S. R., et al., Nerve injury increases GluA2-lacking AMPA receptor prevalence in spinal cords: functional significance and signaling mechanisms. J Pharmacol Exp Ther, 2013. 347(3): p. 765-72.
CLEM, R. L., V. Anggono, and R. L. Huganir, PICK1 regulates incorporation of calcium-permeable AMPA receptors during cortical synaptic strengthening. J Neurosci, 2010. 30(18): p. 6360-6.
CONRAD, K. L., et al., Formation of accumbens GluR2-lacking AMPA receptors mediates incubation of cocaine craving. Nature, 2008. 454(7200): p. 118-21.
DIXON, R. M., J. R. Mellor, and J. G. Hanley, Pick1-mediated glutamate receptor subunit 2 (GLUR2) trafficking contributes to cell death in oxygen/glucose deprived hippocampal neurons. Journal of Biological Chemistry, 2009.
GANGADHARAN, V., et al., Peripheral calcium-permeable AMPA receptors regulate chronic inflammatory pain in mice. J Clin Invest, 2011. 121(4): p. 1608-23.
GARRY, E. M., et al., Specific involvement in neuropathic pain of AMPA receptors and adapter proteins for the GluR2 subunit. Mol.Cell Neurosci., 2003. 24(1): p. 10-22.
HE, J., et al., PICK1 inhibits the E3 ubiquitin ligase activity of Parkin and reduces its neuronal protective effect. Proc Natl Acad Sci USA, 2018. 115(30): p. E7193-E7201.
BISSEN et al. AMPA receptors and their minions: auxiliary proteins in AMPA receptor trafficking. Cell Mol Life Sci. 2019 June; 76(11):2133-2169.
HILL et al. Efficacy and safety of nerinetide for the treatment of acute ischaemic stroke (ESCAPE-NA1): a multicentre, double-blind, randomised controlled trial. Lancet. 2020 Feb. 20. pii: S0140-6736(20)30258-0.
JI, R. R., et al., Central sensitization and LTP: do pain and memory share similar mechanisms? Trends Neurosci, 2003. 26(12): p. 696-705.
KANDASAMY, R. and T. J. Price, The pharmacology of nociceptor priming. Handb Exp Pharmacol, 2015. 227: p. 15-37.
KARLSEN, M. L. et al. Structure of Dimeric and Tetrameric Complexes of the BAR Domain Protein PICK1 Determined by Small-Angle X-Ray Scattering Structure (Oxford, United Kingdom) (2015), 23(7), pp. 1258-1270.
LORGEN, JO., PICK1 facilitates lasting reduction of GluA2 concentration in the hippocampus during chronic epilepsy. Epilepsy Res, 2017. 137: 25-32
LUSCHER, C. and R. C. Malenka, Drug-evoked synaptic plasticity in addiction: from molecular changes to circuit remodeling. Neuron, 2011. 69(4): p. 650-63.
MADSEN, K. L. et al., 2005. Molecular determinants for the complex binding specificity of the PDZ domain in PICK1. Journal of Biological Chemistry 280, 20539-20548.
MARCOTTE, D. J., et al., Lock and chop: A novel method for the generation of a PICK1 PDZ domain and piperidine-based inhibitor co-crystal structure. Protein Sci, 2018. 27(3): p. 672-680.
MOLLER, A. R., Tinnitus and pain. Prog Brain Res, 2007. 166: p. 47-53.
PEKER, S. and A. Sirin, Parallels between phantom pain and tinnitus. Med Hypotheses, 2016. 91: p. 95-97.
RICHNER et al., Hydraulic Extrusion of the Spinal Cord and Isolation of Dorsal Root Ganglia in Rodents, J. Vis. Exp. 2017, 119, 55226.
SOCODATO et al. Calcium-permeable a-Amino-3-hydroxy-5-methyl-4-isoxazolepropionic Acid Receptors Trigger Neuronal Nitric-oxide Synthase Activation to Promote Nerve Cell Death in an Src Kinase-dependent Fashion. J Biol Chem. 2012 Nov. 9; 287(46):38680-94.
SORENSEN et al., 2016, A robust activity marking system for exploring active neuronal ensembles. eLife 2016; 5:e139185.
THOMSEN et al, Computational design of water-soluble alpha-helical barrels, Science, 2014, 346, 485-488.
VANN ESTE, S., W. T. To, and D. De Ridder, Tinnitus and neuropathic pain share a common neural substrate in the form of specific brain connectivity and microstate profiles. Prog Neuropsychopharmacol Biol Psychiatry, 2019. 88: p. 388-400.
VIKMAN, K. S., B. K. Rycroft, and M. J. Christie, Switch to Ca2+-permeable AMPA and reduced NR2B NMDA receptor-mediated neurotransmission at dorsal horn nociceptive synapses during inflammatory pain in the rat. J Physiol, 2008. 586(2): p. 515-27.
WANG, W., et al., Preserved acute pain and impaired neuropathic pain in mice lacking protein interacting with C Kinase 1. Mol Pain, 2011. 7: p. 11.
WOLF, M. E. and C. R. Ferrario, AMPA receptor plasticity in the nucleus accumbens after repeated exposure to cocaine. Neurosci Biobehav Rev, 2010. 35(2): p. 185-211.
WOOLF, C. J. and M. W. Salter, Neuronal plasticity: increasing the gain in pain. Science, 2000. 288(5472): p. 1765-9.
ZACCAI et al. A de novo peptide hexamer with a mutable channel, Nat. Chem. Biol., 2011, 7, 935-941.
ZENG et al. Cell. 2016 Aug. 25; 166(5):1163-1175
ZHANG et al. Protein interacting with C alpha kinase 1 (PICK1) is involved in promoting tumor growth and correlates with poor prognosis of human breast cancer. Cancer Sci. 2010 June; 101(6):1536-42.

1. A polynucleotide comprising a sequence encoding a polypeptide comprising:
a. a first polypeptide part comprising or consisting of an amino acid sequence capable of forming a trimer, tetramer and/or higher order oligomer; and
b. a second polypeptide part comprising or consisting of an amino acid sequence selected from the group consisting of Class I PDZ domains binding motifs (PBM), Class II PBM and Class Ill PBM,
wherein the first and the second polypeptides are optionally operably linked via a linker, for use as a medicament.

2. A polynucleotide comprising a sequence encoding a polypeptide comprising:
a. a first polypeptide part comprising or consisting of an amino acid sequence capable of forming a trimer, tetramer and/or higher order oligomer; and
b. a second polypeptide part comprising or consisting of an amino acid sequence selected from the group consisting of Class I PDZ domains binding motifs (PBM), Class II PBM and Class Ill PBM,
wherein the first and the second polypeptides are optionally operably linked via a linker, for use in the prophylaxis and/or treatment of a disease and/or disorder associated with maladaptive plasticity.

3. The polynucleotide for use according to claim 2, wherein the disease or disorder associated with maladaptive plasticity is pain, drug addiction, amyotrophic lateral sclerosis, epilepsy, tinnitus, migraine, cancer, ischemia, Alzheimer's disease, and/or Parkinson's disease

4. The polynucleotide for use according to any one of claims 1 to 3, wherein the second polypeptide part is consisting of or comprising a Class I PBM comprising an amino acid sequence of Σ-¥ψ, a Class II PBM comprising an amino acid sequence of ψ-¥-ψ, or a Class II PBM comprising an amino acid sequence of ϕ-¥-ψ, wherein
Σ is Thr, Cys or Ser;
¥ is any proteinogenic amino acid;
ψ is any hydrophobic amino acid; and
ϕ is Asp or Glu.

5. The polynucleotide for use according to any one of the preceding claims, wherein the second polypeptide part is selected from the group consisting of HWLKV (SEQ ID NO: 54), IETDV (SEQ ID NO: 55), RRTTPV (SEQ ID NO: 56), and WGESV (SEQ ID NO: 58), preferably HWLKV (SEQ ID NO: 54).

6. The polynucleotide for use according to any one of the preceding claims, wherein the first polypeptide part is an alpha helix, such as an amphipathic helix capable of forming a coiled coil comprising three or more polypeptides according to any one of the preceding claims.

7. The polynucleotide for use according to any one of the preceding claims, wherein the optional linker is a peptide linker, such as a glycine serine (GS) linker.

8. The polynucleotide for use according to any one of the preceding claims, wherein the first polypeptide part is selected from the group consisting of GCN4p1(NQ) (SEQ ID NO: 67), GCN4p1(LI) (SEQ ID NO: 68), GCN4p1(ILI) (SEQ ID NO: 147), CC-Tet (SEQ ID NO: 69), CC-Hex2 (SEQ ID NO: 70), and ATF7-pII (SEQ ID NO: 154), preferably the first polypeptide part is GCN4p1(ILI) (SEQ ID NO: 147) or GCN4p1(LI) (SEQ ID NO: 68).

9. The polynucleotide according to any one of the preceding claims, wherein the first polypeptide part is selected from the group consisting of: SEQ ID NO: 67, 68, 69, 70, 147, 154, and any one of 159-168, the linker is selected from GGS, and any one of SEQ ID NO: 71-74, and the second polypeptide is selected from any one of SEQ ID NO: 5-64 or IIV, IRV, VIV, VRV, and LRV.

10. The polynucleotide according to any one of the preceding claims, wherein the first polypeptide part is selected from the group consisting of: SEQ ID NO: 67, 68, 69, 70, 147, and 154, the linker is SEQ ID NO: 72, and the second polypeptide is selected from any one of SEQ ID NO: 5-64 or IIV, IRV, VIV, VRV, and LRV.

11. The polynucleotide for use according to any one of the preceding claims, wherein the polypeptide is selected from the list consisting of
(SEQ ID NO: 75)
RMKQLEDKVEELLSKQYHLENEVARLKKLVGGGGSHWLKV,
(SEQ ID NO: 77)
RMKQLEDKVEELLSKQYHLENEVARLKKLVGGGGSIETDV,
(SEQ ID NO: 78)
RMKQLEDKVEELLSKQYHLENEVARLKKLVGGGGSRRTTPV,
(SEQ ID NO: 81)
RIKQIEDKIEEILSKIYHIENEIARIKKLIGGGGSHWLKV,
(SEQ ID NO: 83)
RIKQIEDKIEEILSKIYHIENEIARIKKLIGGGGSIETDV,
(SEQ ID NO: 84)
RIKQIEDKIEEILSKIYHIENEIARIKKLIGGGGSRRTTPV,
(SEQ ID NO: 87)
GELAAIKQELAAIKKELAAIKWELAAIKQGGGGSHWLKV,
(SEQ ID NO: 89)
GELAAIKQELAAIKKELAAIKWELAAIKQGGGGSIETDV,
(SEQ ID NO: 90)
GELAAIKQELAAIKKELAAIKWELAAIKQGGGGSRRTTPV,
(SEQ ID NO: 93)
GEIAKSLKEIAKSLKEIAWSLKEIAKSLKGGGGSHWLKV,
(SEQ ID NO: 95)
GEIAKSLKEIAKSLKEIAWSLKEIAKSLKGGGGSIETDV,
(SEQ ID NO: 96)
GEIAKSLKEIAKSLKEIAWSLKEIAKSLKGGGGSRRTTPV,
(SEQ ID NO: 148)
RMKQIEDKLEEILSKLYHIENELARIKKLLGGGGSHWLKV,
(SEQ ID NO: 150)
RMKQIEDKLEEILSKLYHIENELARIKKLLGGGGSIETDV,
(SEQ ID NO: 151)
RMKQIEDKLEEILSKLYHIENELARIKKLLGGGGSRRTTPV,
(SEQ ID NO: 194)
VSSIEKKIEEITSQIIQISNEITLIRNEIAQIKQGGGGSHWLKV,
and
(SEQ ID NO: 195)
VSSIEKKIEEITSQIIQISNEITLIRNEIAQIKQGGGGSIETDV..

12. The polynucleotide for use according to any of the preceding claims, wherein the polynucleotide further comprises a promoter, such as human Synapsin1 promoter.

13. The polynucleotide for use according to any of the preceding claims, wherein the second polypeptide inhibits a PDZ domain containing protein, such as inhibits PICK1, PSD-95, nNOS, Shank1, Shank2, Shank3, Syntenin, GRIP, MAGI1, MAGI2, MAGI3, PSD-93, DLG1, SAP-102, ZO-1, Frizzled, PAR3, or PARE, Mint1, or CASK.

14. The polynucleotide for use according to any of the preceding claims, wherein binding of the second polypeptide part to the PDZ domain of PICK1 result in formation of higher oligomeric states of PICK1, such as trimers, tetramers, pentamers, hexamers, heptamers or octamers of PICK1.

15. The polynucleotide for use according to any of the preceding claims, wherein binding of the second polypeptide part to the PDZ domain of PSD-95 result in liquid-liquid phase separation (LLPS).

16. The polynucleotide for use according to any of the preceding claims, wherein the polynucleotide is comprised in an expression vector.

17. The polynucleotide for use according to any of the preceding claims, wherein the polynucleotide is comprised in an adeno associated vector (AAV).

18. A polynucleotide comprising a sequence encoding a polypeptide comprising:
a. a first polypeptide part comprising or consisting of an amino acid sequence selected from the group consisting of GCN4p1(NQ) (SEQ ID NO: 67), GCN4p1(LI) (SEQ ID NO: 68), GCN4p1(ILI) (SEQ ID NO: 147), CC-Tet (SEQ ID NO: 69), cc-Hex2 (SEQ ID NO: 70), and ATF7-pII (SEQ ID NO: 154); and
b. a second polypeptide part comprising or consisting of an amino acid sequence selected from the group consisting of Class I PDZ domains binding motifs (PBM), Class II PBM and Class III PBM.

19. The polynucleotide according to claim 18, wherein the second polypeptide part is consisting of or comprising a Class I PBM comprising an amino acid sequence of Σ-¥-ψ, a Class II PBM comprising an amino acid sequence of ψ-¥-ψ, or a Class II PBM comprising an amino acid sequence of ϕ-¥-ψ, wherein
Σ is Thr, Cys or Ser;
¥ is any proteinogenic amino acid;
ψ is any hydrophobic amino acid; and
ϕ is Asp or Glu.

20. The polynucleotide according to any one of claims 18 to 19, wherein the second polypeptide part is selected from the group consisting of HWLKV (SEQ ID NO: 54), IETDV (SEQ ID NO: 55), RRTTPV (SEQ ID NO: 56), and WGESV (SEQ ID NO: 58), preferably HWLKV (SEQ ID NO: 54).

21. The polynucleotide according to any one of claims 18 to 20, wherein the optional linker is a glycine serine (GS) linker.

22. The polynucleotide according to any one of claims 18 to 21, wherein the first polypeptide part is selected from the group consisting of GCN4p1(LI) (SEQ ID NO: 68) and GCN4p1(ILI) (SEQ ID NO: 147).

23. The polynucleotide according to any one of claims 18 to 21, wherein the polypeptide is selected from the list consisting of
(SEQ ID NO: 75)
RMKQLEDKVEELLSKQYHLENEVARLKKLVGGGGSHWLKV,
(SEQ ID NO: 77)
RMKQLEDKVEELLSKQYHLENEVARLKKLVGGGGSIETDV,
(SEQ ID NO: 78)
RMKQLEDKVEELLSKQYHLENEVARLKKLVGGGGSRRTTPV,
(SEQ ID NO: 81)
RIKQIEDKIEEILSKIYHIENEIARIKKLIGGGGSHWLKV,
(SEQ ID NO: 83)
RIKQIEDKIEEILSKIYHIENEIARIKKLIGGGGSIETDV,
(SEQ ID NO: 84)
RIKQIEDKIEEILSKIYHIENEIARIKKLIGGGGSRRTTPV,
(SEQ ID NO: 87)
GELAAIKQELAAIKKELAAIKWELAAIKQGGGGSHWLKV,
(SEQ ID NO: 89)
GELAAIKQELAAIKKELAAIKWELAAIKQGGGGSIETDV,
(SEQ ID NO: 90)
GELAAIKQELAAIKKELAAIKWELAAIKQGGGGSRRTTPV,
(SEQ ID NO: 93)
GEIAKSLKEIAKSLKEIAWSLKEIAKSLKGGGGSHWLKV,
(SEQ ID NO: 95)
GEIAKSLKEIAKSLKEIAWSLKEIAKSLKGGGGSIETDV,
(SEQ ID NO: 96)
GEIAKSLKEIAKSLKEIAWSLKEIAKSLKGGGGSRRTTPV,
(SEQ ID NO: 148)
RMKQIEDKLEEILSKLYHIENELARIKKLLGGGGSHWLKV,
(SEQ ID NO: 150)
RMKQIEDKLEEILSKLYHIENELARIKKLLGGGGSIETDV,
(SEQ ID NO: 151)
RMKQIEDKLEEILSKLYHIENELARIKKLLGGGGSRRTTPV,
(SEQ ID NO: 194)
VSSIEKKIEEITSQIIQISNEITLIRNEIAQIKQGGGGSHWLKV,
and
(SEQ ID NO: 195)
VSSIEKKIEEITSQIIQISNEITLIRNEIAQIKQGGGGSIETDV.

24. The polynucleotide according to any of claims 18 to 23, wherein the polynucleotide further comprises a promoter.

25. The polynucleotide according to claim 24, wherein the promoter is human Synapsin1 promoter.

26. The polynucleotide according to any of claims 18 to 25, wherein the second polypeptide inhibits a PDZ domain containing protein, such as inhibits PICK1, PSD-95, nNOS, Shank1, Shank2, Shank3, Syntenin, GRIP, MAGI1, MAGI2, MAGI3, PSD-93, DLG1, SAP-102, ZO-1, Frizzled, PAR3, or PARE, Mint1, or CASK.

27. The polynucleotide according to any of claims 18 to 26, wherein binding of the second polypeptide part to the PDZ domain of PICK1 results in formation of higher oligomeric states of PICK1, such as trimers, tetramers, pentamers, hexamers, heptamers or octamers of PICK1.

28. The polynucleotide according to any of claims 18 to 26, wherein binding of the second polypeptide part to the PDZ domain of PSD-95 results in liquid-liquid phase separation (LLPS).

29. An expression vector comprising the polynucleotide according to any of claims 18 to 28, such as an adeno associated vector (AAV).

30. A polypeptide encoded by the polynucleotide, or the expression vector according to any one of claims 18 to 29.
